(19) United States
(12) Patent Application Publication
(10) Pub. No.: US 2026/0000758 A1
(43) Pub. Date: Jan. 1, 2026

(54) BISPECIFIC ANTIGEN BINDING MOLECULES THAT BIND GDF8 AND ACTIVIN A AND USES THEREOF

(71) Applicant: Regeneron Pharmaceuticals, Inc., Tarrytown, NY (US)

(72) Inventor: David GLASS, Briarcliff Manor, NY (US)

(21) Appl. No.: 19/251,109

(22) Filed: Jun. 26, 2025

Related U.S. Application Data
(60) Provisional application No. 63/665,593, filed on Jun. 28, 2024.

(51) Int. Cl. A61K 39/395; A61K 39/00; A61P 3/04; C07K 16/22

(52) U.S. Cl. CPC A61K 39/3955; A61P 3/04; C07K 16/22; A61K 2039/507; A61K 2039/545; C07K 2317/24; C07K 2317/31; C07K 2317/92

(57) ABSTRACT

The present invention is related to bispecific antigen binding molecules that specifically bind to Activin A and GDF8, and therapeutic and diagnostic methods of using those molecules.

### CROSS-REFERENCE TO RELATED APPLICATIONS

This application claims the benefit of U.S. Provisional Application No. 63/665,593, filed Jun. 28, 2024, the disclosure of which is incorporated herein in its entirety.

### REFERENCE TO A SEQUENCE LISTING

The instant application contains a Sequence Listing, which has been submitted electronically in XML format and is hereby incorporated by reference in its entirety. Said XML file, created on Jun. 27, 2024, is named Sequence-Listing-40848-0121USP1.xml and is 20,109 bytes in size.

### FIELD OF THE INVENTION

The present invention is related to bispecific antigen binding molecules that specifically bind to Activin A and GDF8, and therapeutic and diagnostic methods of using those molecules.

### BACKGROUND OF THE INVENTION

Growth and differentiation factor-8 (GDF8, also known as myostatin), is a secreted ligand belonging to the transforming growth factor-β (TGF-β) superfamily of growth factors. GDF8 plays a central role in the development and maintenance of skeletal muscle, acting as a negative regulator of muscle mass. While the myostatin null mouse phenotype demonstrates the importance of GDF8 in the control of muscle size during development, muscle hypertrophy can also be elicited in adult muscle through inhibition of GDF8 with neutralizing antibodies, decoy receptors, or other antagonists. Administration of GDF8 neutralizing antibodies has been reported to result in muscle mass increases of between 10 and 30%. The increased muscle mass seen is due to increased fiber diameter as opposed to myofiber hyperplasia (fiber number). A number of studies have also reported increases in muscle strength or performance commensurate with increased size including twitch and tetanic force. Use of a cleavage resistant version of the GDF8 propeptide also leads to increased muscle size.

Other GDF8 antagonists have been used in adult mice with significant effects on skeletal muscle mass. These include the extracellular portion of the Type II GDF8 receptor, ActRIIB, stabilized by fusion to an IgG Fc domain (“ActRIIB-Fc”). The clinical molecule “ACE-031” is an example of an ActRIIB-Fc molecule.

Although ActRIIB-Fc has been shown to increase muscle mass in experimental animals, in human clinical trials this molecule was shown to cause various adverse side effects. For example, administration of ACE-031 to postmenopausal women in a Phase Ib ascending dose study was shown to cause undesired increases in hemoglobin and decreases in FSH levels. In addition, a Phase II study of ACE-031 in pediatric patients with muscular dystrophy was discontinued due to adverse effects including nose and gum bleeding. Dilated blood vessels are also observed in patients treated with ActRIIB-Fc.

Experiments have shown that the muscle growth-inducing effects of ActRIIB-Fc are attenuated but not eliminated in myostatin null mice, suggesting that ActRIIB-Fc exerts its muscle mass-inducing effects by antagonizing other ActRIIB ligand(s) in addition to GDF8. Other ligands that bind ActRIIB include Activin A, Activin B, Activin AB, Inhibin A, Inhibin B, GDF3, GDF11, Nodal, BMP2, BMP4, and BMP7.

Activins also belong to the transforming growth factor-beta (TGF-β) superfamily and exert a broad range of biological effects on cell proliferation, differentiation, and apoptosis. Activins are homo- or heterodimers of InhibinβA, InhibinβB, InhibinβC and InhibinβE, different combinations of which create the various members of the activin protein group. For example, Activin A is a homodimer of InhibinβA and Activin B is a homodimer of InhibinβB, whereas Activin AB is a heterodimer of InhibinβA and InhibinβB and Activin AC is a heterodimer of InhibinβA and InhibinβC (Tsuchida, K. et al., Cell Commun Signal 7:15 (2009)).

Activin A binds to and activates receptor complexes on the surface of cells known as Activin Type II receptors (Type IIA and Type IIB, also known as ActRIIA and ActRIIB, respectively). The activation of these receptors leads to the phosphorylation of an Activin Type I receptor (e.g., Alk4 or 7), which in turn leads to the phosphorylation of SMAD 2 and 3 proteins, the formation of SMAD complexes (with SMAD4), and the translocation of the SMAD complex to the cell nucleus, where SMAD2 and SMAD3 function to regulate transcription of various genes (Sozzani, S. and Musso, T., Blood 117(19):5013-5015 (2011)).

Numerous other ligands bind to and activate ActRIIB, including GDF8 (myostatin), Activin B, Activin AB, Inhibin A, Inhibin B, GDF3, GDF11, Nodal, BMP2, BMP4, BMP7, BMP9, and BMP10. Blocking the interactions of ActRIIB with its ligands can lead to beneficial physiological effects. For example, GDF8 plays a central role in the development and maintenance of skeletal muscle, acting as a negative regulator of muscle mass (McPherron A C et al. (1997). Nature 387(6628):83-90).

Administration of ActRIIB-Fc (i.e., the extracellular portion of the Type llB receptor, ActRIIB, stabilized by fusion to an IgG Fc domain) leads to significant increases in skeletal muscle mass and improves muscle weight and measurements of muscle strength in mice (Lee S J, et al. (2005) Proc Natl Acad Sci USA 102(50):18117-18122). The efficacy of ActRIIB-Fc is attenuated but not eliminated in Mstn (myostatin) null mice, demonstrating that other ActRIIB ligand(s) in addition to myostatin can function as negative regulators of muscle growth. Thus, a need exists for additional inhibitors of ActRIIB signaling that can provide clinical benefits.

### BRIEF SUMMARY OF THE INVENTION

The present disclosure provides polyspecific antigen-binding molecules comprising a first antigen-binding domain (D1) that binds GDF8; and a second antigen-binding domain (D2) that binds Activin A.

The present disclosure provides bispecific antigen-binding molecules comprising a first antigen-binding domain (D1) that binds GDF8; and a second antigen-binding domain (D2) that binds inhibin βA and dimers containing inhibin RA, e.g., Activin A, Activin AB, etc.

The bispecific antigen-binding molecules and bi-specific antibodies of the invention are useful, inter alia, for inhibiting Activin A-mediated signaling and inhibiting GDF8-mediated signaling, producing beneficial clinical outcomes through the inhibition of Activin A-mediated signaling and GDF8-mediated signaling, e.g., for treating diseases and disorders caused by or related to Activin A activity and/or signaling, and/or GDF8 activity and/or signaling.

The bi-specific antibodies of the invention also have utility for use in conjunction with inhibitors of other ligands of the ActRIIA and ActRIIB receptors, such as GDF8 inhibitors.

The present invention provides bispecific antibodies and antigen-binding fragments thereof that can specifically bind to an Activin A and inhibit Activin A-mediated signal transduction and specifically bind to GDF8 and inhibit GDF8-mediated signal transduction.

In certain embodiments, the GDF8 x Activin A bispecific antibodies are fully human antibodies that bind to Activin A and GDF8 with high affinity and inhibit Activin A-mediated signal transduction and GDF8-mediated signal transduction.

The antibodies of the present invention are useful, inter alia, for deactivating or decreasing the activity of GDF8 and Activin A proteins.

In certain embodiments, the antibodies are useful in preventing, treating or ameliorating at least one symptom or indication of a GDF8-associated disease or disorder in a subject and/or Activin A-associated disease or disorder in a subject.

In certain embodiments, the antibodies may be administered prophylactically or therapeutically to a subject having or at risk of having a GDF8-associated disease or disorder and/or an Activin A-associated disease or disorder.

In specific embodiments, the antibodies are used in the prevention and treatment of sarcopenia, cachexia (either idiopathic or secondary to other conditions, e.g., cancer, chronic renal failure, or chronic obstructive pulmonary disease), muscle injury, muscle wasting and muscle atrophy, e.g., muscle atrophy or wasting caused by or associated with disuse, immobilization, bed rest, injury, medical treatment or surgical intervention (e.g., hip fracture, hip replacement, knee replacement, etc.) or by necessity of mechanical ventilation when administered to a subject in need thereof.

In some embodiments, the antibodies of the invention bind to an Activin A protein and a GDF8 protein. Further, the antibodies disclosed herein bind to an Activin A protein and a GDF8 protein with high affinity. Full length GDF8 used in the present invention include GDF8 proteins which may be derived from an animal such as a human or a mouse. For example, the full-length amino acid sequence of GDF8 is available with reference to UniProtKB/Swiss-Prot accession number 008689) (SEQ ID NO: 39). In some cases, the antibodies of the invention may bind to a GDF8 protein or fragment thereof such as SEQ ID NO: 42. Full length, native Activin A used in the present invention include Activin A proteins which may be derived from a mammal such as a human or a mouse. For example, the amino acid sequence of human Activin A is available with reference to UniProtKB/Swiss-Prot accession number P08476) (SEQ ID NO: 40). In some cases, the antibodies of the invention may bind to a recombinant Activin A protein or fragment thereof such as protein or an appropriate fragment thereof such as Gly311-Ser426 of UniProtKB/Swiss-Prot accession number P08476 (SEQ ID NO: 43) which may be expressed in E. coli or in any other eukaryotic or mammalian cells such as Chinese hamster ovary (CHO) cells.

The antibodies of the invention can be full-length (for example, an IgG1 or IgG4 antibody format) or may comprise only an antigen-binding portion (for example, a Fab, F(ab′)₂ or scFv fragment), and may be modified to affect functionality, e.g., to increase persistence in the host or to eliminate residual effector functions (Reddy et al., 2000, J. Immunol. 164:1925-1933). In certain embodiments, the antibodies may be bispecific.

In a first aspect, the present invention provides isolated recombinant bispecific antibodies or antigen-binding fragments thereof that bind specifically to a GDF8 protein and an Activin A protein. In some embodiments, the antibodies are fully human monoclonal antibodies.

In some cases, the bispecific antibodies or antigen-binding fragments thereof comprise (i) the CDRs of an HCVR of an antibody that binds GDF8; (ii) the CDRs of an HCVR of an antibody that binds Activin A; and (iii) the CDRs of an LCVR of an antibody that binds GDF8. In some cases, the HCVR of an antibody that binds GDF8 comprises an amino acid sequence selected from the group consisting of sequences listed in Table 1; the HCVR of an antibody that binds Activin A comprises an amino acid sequence selected from the group consisting of sequences listed in Table 1; and/or the LCVR of an antibody that binds GDF8 comprises an amino acid sequence selected from the group consisting of sequences listed in Table 1.

Exemplary GDF8 x Activin A bispecific antibodies of the present invention comprise the amino acid sequences and nucleic acid sequences listed in Tables 1, 2, and 3 herein. Table 1 sets forth the amino acid sequence identifiers of the heavy chain variable regions (HCVRs), light chain variable regions (LCVRs), heavy chain complementarity determining regions (HCDRs) (HCDR1, HCDR2 and HCDR3), and light chain complementarity determining regions (LCDRs) (LCDR1, LCDR2 and LCDR3) of exemplary antibodies. Table 2 sets forth the nucleic acid sequence identifiers of the HCVRs, LCVRs, HCDR1, HCDR2 HCDR3, LCDR1, LCDR2 and LCDR3 of the exemplary antibodies. Table 3 sets forth the heavy chain and light chain amino acid sequences and nucleic acid sequences of the exemplary antibodies.

The present invention provides antibodies, or antigen-binding fragments thereof, comprising an HCVR comprising an amino acid sequence selected from any of the HCVR amino acid sequences listed in Table 1, or a substantially similar sequence thereof having at least 90%, at least 95%, at least 98% or at least 99% sequence identity thereto.

The present invention also provides antibodies, or antigen-binding fragments thereof, comprising an LCVR comprising an amino acid sequence selected from any of the LCVR amino acid sequences listed in Table 1, or a substantially similar sequence thereof having at least 90%, at least 95%, at least 98% or at least 99% sequence identity thereto.

The present invention also provides antibodies, or antigen-binding fragments thereof, comprising an HCVR and an LCVR amino acid sequence pair (HCVR/LCVR) comprising any of the HCVR amino acid sequences listed in Table 1 paired with any of the LCVR amino acid sequences listed in Table 1. According to certain embodiments, the present invention provides antibodies, or antigen-binding fragments thereof, comprising an HCVR/LCVR amino acid sequence pair contained within any of the exemplary antibodies listed in Table 1. The present invention provides bispecific antibodies, or antigen-binding fragments thereof comprising a first heavy chain variable region (HCVR) having an amino acid sequence of SEQ ID NO: 2, or a substantially similar sequence thereof having at least 90%, at least 95%, at least 98% or at least 99% sequence identity, and a second HCVR having an amino acid sequence selected from the group consisting of SEQ ID NO: 10 and SEQ ID NO: 32, or a substantially similar sequence thereof having at least 90%, at least 95%, at least 98% or at least 99% sequence identity.

The present invention also provides a bispecific antibody or antigen-binding fragment of an antibody comprising a light chain variable region (LCVR) having an amino acid sequence of SEQ ID NO: 18, or a substantially similar sequence thereof having at least 90%, at least 95%, at least 98% or at least 99% sequence identity.

In some cases, the bispecific antibody of the invention comprises a first HCVR, a second HCVR, and a common light chain LCVR.

In some cases, the bispecific antibody comprises a GDF8-specific binding domain and an Activin A-specific binding domain, wherein the GDF8-specific binding domain comprises the CDRs of a HCVR having an amino acid sequence consisting of SEQ ID NO: 2; and the CDRs of a LCVR having an amino acid sequence consisting of SEQ ID NO: 18; and wherein the Activin A-specific binding domain comprises the CDRs of a HCVR having an amino acid sequence selected from the group consisting of SEQ ID NOs: 10 and 32; and the CDRs of a LCVR having an amino acid sequence consisting of SEQ ID NO: 18.

The present invention also provides an multi specific antibody or antigen-binding fragment thereof comprising two or more HCVR and LCVR (HCVR/LCVR) amino acid sequence pairs selected from the group consisting of SEQ ID NO: 2/18, 10/18, and 32/18. In some cases, the bispecific antibody comprises two HCVR/LCVR amino acid sequence pairs of SEQ ID NO: 2/18 and 10/18 (e.g., H4H11418D). In some cases, the bispecific antibody comprises two HCVR/LCVR amino acid sequence pairs of SEQ ID NO: 2/18 and 32/18 (e.g., H4H11419D).

The present invention also provides an antibody or antigen-binding fragment of an antibody comprising a heavy chain CDR3 (HCDR3) domain having an amino acid sequence selected from the group consisting of SEQ ID NO: 8, 16, and 36, or a substantially similar sequence thereof having at least 90%, at least 95%, at least 98% or at least 99% sequence identity; and a light chain CDR3 (LCDR3) domain having the amino acid sequence of SEQ ID NO: 18, or a substantially similar sequence thereof having at least 90%, at least 95%, at least 98% or at least 99% sequence identity.

In certain embodiments, the antibody or antigen-binding portion of an antibody comprises a HCDR3/LCDR3 amino acid sequence pair selected from the group consisting of SEQ ID NO:8/24, 16/24, and 36/24.

The present invention also provides an antibody or fragment thereof further comprising a heavy chain CDR1 (HCDR1) domain having an amino acid sequence selected from the group consisting of SEQ ID NO: 4 and 12, or a substantially similar sequence thereof having at least 90%, at least 95%, at least 98% or at least 99% sequence identity; a heavy chain CDR2 (HCDR2) domain having an amino acid sequence selected from the group consisting of SEQ ID NO: 6, 14, 34, or a substantially similar sequence thereof having at least 90%, at least 95%, at least 98% or at least 99% sequence identity; a light chain CDR1 (LCDR1) domain having an amino acid sequence of SEQ ID NO: 20, or a substantially similar sequence thereof having at least 90%, at least 95%, at least 98% or at least 99% sequence identity; and a light chain CDR2 (LCDR2) domain having an amino acid sequence of SEQ ID NO: 22, or a substantially similar sequence thereof having at least 90%, at least 95%, at least 98% or at least 99% sequence identity.

Certain non-limiting, exemplary antibodies and antigen-binding fragments of the invention comprise HCDR1-HCDR2-HCDR3-LCDR1-LCDR2-LCDR3 domains, respectively, having the amino acid sequences selected from the group consisting of: SEQ ID NOs: 4-6-8-20-22-24; 12-14-16-20-22-24; and 12-34-36-20-22-24. In some cases, the bispecific antibody of the invention comprises HCDR1-HCDR2-HCDR3-LCDR1-LCDR2-LCDR3 domains, respectively, having the amino acid sequences of 4-6-8-20-22-24 and 12-14-16-20-22-24 (e.g., H4H11418D). In some cases, the bispecific antibody of the invention comprises HCDR1-HCDR2-HCDR3-LCDR1-LCDR2-LCDR3 domains, respectively, having the amino acid sequences of 4-6-8-20-22-24 and 12-34-36-20-22-24 (e.g., H4H11419D).

In a related embodiment, the invention includes an antibody or antigen-binding fragment of an antibody which specifically binds GDF8 and/or Activin A proteins, wherein the antibody or fragment comprises the heavy and light chain CDR domains contained within heavy and light chain variable region (HCVR/LCVR) sequences selected from the group consisting of SEQ ID NO: 2/18, 10/18, and 32/18.

The present invention also provides antibodies, or antigen-binding fragments thereof, comprising a HCVR and a LCVR, said HCVR comprising an amino acid sequence listed in Table 1 having no more than twelve amino acid substitutions, and/or said LCVR comprising an amino acid sequence listed in Table 1 having no more than ten amino acid substitutions. For example, the present invention provides antibodies or antigen-binding fragments thereof comprising a HCVR and a LCVR, said HCVR comprising an amino acid sequence listed in Table 1, said amino acid sequence having one, two, three, four, five, six, seven, eight, nine, ten, eleven or twelve amino acid substitutions. In another example, the present invention provides antibodies or antigen-binding fragments thereof comprising a HCVR and a LCVR, said LCVR comprising an amino acid sequence listed in Table 1, said amino acid sequence having one, two, three, four, five, six, seven, eight, nine or ten amino acid substitutions. In one embodiment, the present invention provides GDF8 x Activin A bispecific antibodies or antigen-binding fragments thereof comprising a HCVR and a LCVR, said HCVR comprising an amino acid sequence listed in Table 1, said amino acid sequence having at least one amino acid substitution, and/or said LCVR comprising an amino acid sequence listed in Table 1, said amino acid sequence having at least one amino acid substitution.

The present invention also provides antibodies, or antigen-binding fragments thereof, comprising a heavy chain CDR1 (HCDR1) comprising an amino acid sequence selected from any of the HCDR1 amino acid sequences listed in Table 1 or a substantially similar sequence thereof having at least 90%, at least 95%, at least 98% or at least 99% sequence identity.

The present invention also provides antibodies, or antigen-binding fragments thereof, comprising a heavy chain CDR2 (HCDR2) comprising an amino acid sequence selected from any of the HCDR2 amino acid sequences listed in Table 1 or a substantially similar sequence thereof having at least 90%, at least 95%, at least 98% or at least 99% sequence identity.

The present invention also provides antibodies, or antigen-binding fragments thereof, comprising a heavy chain CDR3 (HCDR3) comprising an amino acid sequence selected from any of the HCDR3 amino acid sequences listed in Table 1 or a substantially similar sequence thereof having at least 90%, at least 95%, at least 98% or at least 99% sequence identity.

The present invention also provides antibodies, or antigen-binding fragments thereof, comprising a light chain CDR1 (LCDR1) comprising an amino acid sequence selected from any of the LCDR1 amino acid sequences listed in Table 1 or a substantially similar sequence thereof having at least 90%, at least 95%, at least 98% or at least 99% sequence identity.

The present invention also provides antibodies, or antigen-binding fragments thereof, comprising a light chain CDR2 (LCDR2) comprising an amino acid sequence selected from any of the LCDR2 amino acid sequences listed in Table 1 or a substantially similar sequence thereof having at least 90%, at least 95%, at least 98% or at least 99% sequence identity.

The present invention also provides antibodies, or antigen-binding fragments thereof, comprising a light chain CDR3 (LCDR3) comprising an amino acid sequence selected from any of the LCDR3 amino acid sequences listed in Table 1 or a substantially similar sequence thereof having at least 90%, at least 95%, at least 98% or at least 99% sequence identity.

The present invention also provides antibodies, or antigen-binding fragments thereof, comprising an HCDR3 and an LCDR3 amino acid sequence pair (HCDR3/LCDR3) comprising any of the HCDR3 amino acid sequences listed in Table 1 paired with any of the LCDR3 amino acid sequences listed in Table 1.

According to certain embodiments, the present invention provides antibodies, or antigen-binding fragments thereof, comprising an HCDR3/LCDR3 amino acid sequence pair contained within any of the exemplary GDF8 x Activin A bispecific antibodies listed in Table 1.

In certain embodiments, the HCDR3/LCDR3 amino acid sequence pair is selected from the group consisting of SEQ ID NOs: 8/24 and 16/24 (e.g., H4H11418D), and SEQ ID NOs: 8/24 and 36/24 (e.g., H4H11419D).

The present invention also provides antibodies, or antigen-binding fragments thereof, comprising a HCVR and a LCVR, said HCVR comprising HCDR1 comprising an amino acid sequence differing from an amino acid sequence listed in Table 1 by 1 amino acid, HCDR2 comprising an amino acid sequence differing from an amino acid sequence listed in Table 1 by 1 amino acid, and/or HCDR3 comprising an amino acid sequence differing from an amino acid sequence listed in Table 1 by 1 amino acid. In certain embodiments, the present invention provides antibodies, or antigen-binding fragments thereof, comprising a HCVR and a LCVR, said LCVR comprising LCDR1 comprising an amino acid sequence differing from an amino acid sequence listed in Table 1 by 1 amino acid, LCDR2 comprising an amino acid sequence differing from an amino acid sequence listed in Table 1 by 1 amino acid, and/or LCDR3 comprising an amino acid sequence differing from an amino acid sequence listed in Table 1 by 1 amino acid.

For example, the present invention provides antibodies, or antigen-binding fragments thereof, comprising a HCVR and a LCVR, said HCVR comprising HCDR1 comprising an amino acid sequence selected from the group consisting of SEQ ID NO: 4 and 12 or an amino acid sequence differing from SEQ ID NO: 4 and/or 12 by 1 amino acid, HCDR2 comprising an amino acid sequence selected from the group consisting of SEQ ID NO: 6, 14, and 34 or an amino acid sequence differing from SEQ ID NO: 6, 14, and/or 34 by 1 amino acid, and HCDR3 comprising an amino acid sequence selected from the group consisting of SEQ ID NO: 8, 16, and 36 or an amino acid sequence differing from SEQ ID NO: 8, 16, and/or by 1 amino acid.

In another exemplary embodiment, the present invention provides antibodies, or antigen-binding fragments thereof, comprising a HCVR and a LCVR, said LCVR comprising LCDR1 comprising an amino acid sequence of SEQ ID NO: 20 or an amino acid sequence differing from SEQ ID NO: 20 by 1 amino acid, LCDR2 comprising an amino acid sequence of SEQ ID NO: 22 or an amino acid sequence differing from SEQ ID NO: 22 by 1 amino acid, and/or LCDR3 comprising an amino acid sequence of SEQ ID NO: 24 or an amino acid sequence differing from SEQ ID NO: 24 by 1 amino acid.

The present invention also provides bispecific antibodies, or antigen-binding fragments thereof, comprising a set of six CDRs (i.e., HCDR1-HCDR2-HCDR3-LCDR1-LCDR2-LCDR3) contained within any of the exemplary antibodies listed in Table 1. In certain embodiments, the HCDR1-HCDR2-HCDR3-LCDR1-LCDR2-LCDR3 amino acid sequence set is selected from the group consisting of SEQ ID NOs: 4-6-8-20-22-24, 12-14-16-20-22-24, and 12-34-36-20-22-24.

In a related embodiment, the present invention provides antibodies, or antigen-binding fragments thereof, comprising a set of six CDRs (i.e., HCDR1-HCDR2-HCDR3-LCDR1-LCDR2-LCDR3) contained within an HCVR/LCVR amino acid sequence pair as defined by any of the exemplary antibodies listed in Table 1. For example, the present invention includes antibodies, or antigen-binding fragments thereof, comprising the HCDR1-HCDR2-HCDR3-LCDR1-LCDR2-LCDR3 amino acid sequences set contained within an HCVR/LCVR amino acid sequence pair selected from the group consisting of SEQ ID NO: 2/18, 10/18, and 32/18.

Methods and techniques for identifying CDRs within HCVR and LCVR amino acid sequences are well known in the art and can be used to identify CDRs within the specified HCVR and/or LCVR amino acid sequences disclosed herein. Exemplary conventions that can be used to identify the boundaries of CDRs include, e.g., the Kabat definition, the Chothia definition, and the AbM definition. In general terms, the Kabat definition is based on sequence variability, the Chothia definition is based on the location of the structural loop regions, and the AbM definition is a compromise between the Kabat and Chothia approaches. See, e.g., Kabat, “Sequences of Proteins of Immunological Interest,” National Institutes of Health, Bethesda, Md. (1991); Al-Lazikani et al., J. Mol. Biol. 273:927-948 (1997); and Martin et al., Proc. Natl. Acad. Sci. USA 86:9268-9272 (1989). Public databases are also available for identifying CDR sequences within an antibody.

In certain embodiments, the present invention includes a bispecific antibody or antigen-binding fragment thereof that binds specifically to GDF8 and Activin A, wherein the antibody or antigen-binding fragment thereof comprises three heavy chain complementarity determining regions (CDRs) (HCDR1, HCDR2 and HCDR3) contained within a heavy chain variable region (HCVR) and three light chain CDRs (LCDR1, LCDR2 and LCDR3) contained within a light chain variable region (LCVR), wherein the HCVR comprises: (i) an amino acid sequence selected from the group consisting of SEQ ID NOs: 2, 10, and 32; (ii) an amino acid sequence having at least 90% identity to the amino acid sequence selected from the group consisting of SEQ ID NOs: 2, 10, and 32; (iii) an amino acid sequence having at least 95% identity to the amino acid sequence selected from the group consisting of SEQ ID NOs: 2, 10, and 32; or (iv) an amino acid sequence selected from the group consisting of SEQ ID NOs: 2, 10, and 32, said amino acid sequence having no more than 12 amino acid substitutions; and the LCVR comprises: (a) an amino acid sequence of SEQ ID NO: 18; (b) an amino acid sequence having at least 90% identity to the amino acid sequence of SEQ ID NO: 18; (c) an amino acid sequence having at least 95% identity to the amino acid sequence selected of SEQ ID NO: 18; or (d) an amino acid sequence of SEQ ID NO: 18, said amino acid sequence having no more than 10 amino acid substitutions.

In certain preferred embodiments, the present invention includes bispecific antibodies or antigen-binding fragments thereof that bind specifically to GDF8 in an antagonist manner, i.e., decrease or block GDF8 binding and/or activity.

In certain preferred embodiments, the present invention includes bispecific antibodies or antigen-binding fragments thereof that bind specifically to Activin A in an antagonist manner, i.e., decrease or block Activin A binding and/or activity.

In certain preferred embodiments, the present invention includes bispecific antibodies or antigen-binding fragments thereof that bind specifically to GDF8 and Activin A in an antagonist manner, i.e., decrease or block GDF8 and Activin A binding and/or activity.

In some embodiments, the bispecific antibodies or antigen-binding fragments of the present invention increase total lean mass in a subject. In some embodiments, the bispecific antibodies or antigen-binding fragments of the present invention increase total lean mass by Echo MRI. In some cases, the bispecific antibodies or antigen-binding fragments of the present invention increase total lean mass compared to baseline. In some cases, the bispecific antibodies or antigen-binding fragments of the present invention increase total lean mass compared to isotype control. In some embodiments, the bispecific antibodies or antigen-binding fragments of the present invention increase lean body mass in a subject. In some embodiments, the bispecific antibodies or antigen-binding fragments of the present invention do not increase heart weight. In some embodiments, the bispecific antibodies or antigen-binding fragments of the present invention do not decrease liver weight.

In some embodiments, the bispecific antibodies or antigen-binding fragments of the present invention increase skeletal muscle weight. In some cases, the skeletal muscle is selected from the group consisting of tibialis anterior (TA), quadriceps muscle, and gastrocnemius muscle (GA).

In some embodiments, the bispecific antibodies or antigen-binding fragments of the present invention decrease total body fat in a subject. In some embodiments, the bispecific antibodies or antigen-binding fragments of the present invention decrease total body fat in a subject by Echo MRI. In some cases, the bispecific antibodies or antigen-binding fragments of the present invention decrease total body fat in a subject compared to baseline. In some cases, the bispecific antibodies or antigen-binding fragments of the present invention decrease total body fat in a subject compared to isotype control. In some cases, the bispecific antibodies or antigen-binding fragments of the present invention decrease total body fat in a subject and do not decrease liver weight in the subject. In some cases, the bispecific antibodies or antigen-binding fragments of the present invention decrease total body fat in a subject and increase lean body mass in the subject.

In some cases, the bispecific antibodies or antigen-binding fragments of the present invention decrease total body fat in a subject, increase lean body mass in the subject, and do not decrease liver weight in the subject.

A method for increasing lean body mass, muscle mass, or strength in a subject is provided, the method comprising administering to the subject an antigen-binding molecule comprising: a GDF8-specific binding domain; an Activin A-specific binding domain; and optionally a common light chain. In some cases, (i) the GDF8-specific binding domain comprises three HCDRs of an HCVR of an antibody that binds GDF8; (ii) the Activin A-specific binding domain comprises three HCDRs of an HCVR of an antibody that binds Activin A; and (iii) the common light chain comprises three LCDRs of an LCVR of an antibody that binds GDF8. In some cases, the three HCDRs of the HCVR of an antibody that binds GDF8 comprises an amino acid sequence selected from the group consisting of sequences listed in Table 1; the three HCDRs of the HCVR of an antibody that binds Activin A comprises an amino acid sequence selected from the group consisting of sequences listed in Table 1; and/or the three LCDRs of an LCVR of an antibody that binds GDF8 comprises an amino acid sequence selected from the group consisting of sequences listed in Table 1. In some cases, the method does not decrease liver weight in the subject.

A method for decreasing total body fat in a subject is provided, the method comprising administering to the subject an antigen-binding molecule comprising: a GDF8-specific binding domain; an Activin A-specific binding domain; and optionally a common light chain. In some cases, (i) the GDF8-specific binding domain comprises three HCDRs of an HCVR of an antibody that binds GDF8; (ii) the Activin A-specific binding domain comprises three HCDRs of an HCVR of an antibody that binds Activin A; and (iii) the common light chain comprises three LCDRs of an LCVR of an antibody that binds GDF8. In some cases, the three HCDRs of the HCVR of an antibody that binds GDF8 comprises an amino acid sequence selected from the group consisting of sequences listed in Table 1; the three HCDRs of the HCVR of an antibody that binds Activin A comprises an amino acid sequence selected from the group consisting of sequences listed in Table 1; and/or the three LCDRs of an LCVR of an antibody that binds GDF8 comprises an amino acid sequence selected from the group consisting of sequences listed in Table 1. In some cases, the method does not decrease liver weight in the subject.

The present invention includes GDF8 x Activin A bispecific antibodies having a modified glycosylation pattern. In some embodiments, modification to remove undesirable glycosylation sites may be useful, or an antibody lacking a fucose moiety present on the oligosaccharide chain, for example, to increase antibody dependent cellular cytotoxicity (ADCC) function (see Shield et al. (2002) JBC 277:26733). In other applications, modification of galactosylation can be made in order to modify complement dependent cytotoxicity (CDC).

The present invention also provides for antibodies and antigen-binding fragments thereof that compete for specific binding to GDF8 and/or Activin A with an antibody or antigen-binding fragment thereof comprising the CDRs of a HCVR and the CDRs of a LCVR, wherein the HCVR and LCVR each has an amino acid sequence selected from the HCVR and LCVR sequences listed in Table 1.

The present invention also provides antibodies and antigen-binding fragments thereof that cross-compete for binding to GDF8 and/or Activin A with a reference antibody or antigen-binding fragment thereof comprising the CDRs of a HCVR and the CDRs of a LCVR, wherein the HCVR and LCVR each has an amino acid sequence selected from the HCVR and LCVR sequences listed in Table 1.

The present invention also provides antibodies and antigen-binding fragments thereof that bind to the same epitope as a reference antibody or antigen-binding fragment thereof comprising three CDRs of a HCVR and three CDRs of a LCVR, wherein the HCVR and LCVR each has an amino acid sequence selected from the HCVR and LCVR sequences listed in Table 1.

The present invention also provides isolated bispecific antibodies and antigen-binding fragments thereof that inhibit ligand-induced signaling by GDF8, Activin A or a combination of GDF8 and Activin A from forming a signaling complex with an Activin type II receptor.

In some embodiments, the GDF8 x Activin A bispecific antibody or antigen-binding fragment thereof prevents GDF8 and/or Activin A from forming signaling complex with an Activin type II receptor.

In certain embodiments, the antibodies or antigen-binding fragments of the present invention are multispecific comprising a first binding specificity to a first epitope of Activin A and a second binding specificity to a second epitope of GDF8.

The present invention provides isolated GDF8 x Activin A multispecific antibodies and antigen-binding fragments thereof that may bind to the same epitope on Activin A or GDF8 or may bind to a different epitope on Activin A or GDF8.

In certain embodiments, the antibodies or antigen-binding fragments of the present invention are multispecific comprising a first binding specificity to a first epitope of Activin A and a second binding specificity to a second epitope of Activin A wherein the first and second epitopes are distinct and non-overlapping.

In certain embodiments, the antibodies or antigen-binding fragments of the present invention are multispecific comprising a first binding specificity to a first epitope of GDF8 and a second binding specificity to a second epitope of GDF8 wherein the first and second epitopes are distinct and non-overlapping.

In certain embodiments, the present invention provides an isolated bispecific antibody or antigen-binding fragment thereof that has one or more of the following characteristics:
(a) is a fully human monoclonal antibody;
(b) binds to GDF8 protein Asp268-Ser376 of UniProtKB/Swiss-Prot accession number 008689 (SEQ ID NO: 42) at 25° C. with a dissociation constant (K_{D}) of less than about 100 pM, less than about 70 pM, less than about 50 pM, less than about 25 pM, less than about 20 pM, or less than about 15 pM, as measured in a surface plasmon resonance assay;
(c) binds to GDF8 protein Asp268-Ser376 of UniProtKB/Swiss-Prot accession number 008689 (SEQ ID NO: 42) at 37° C. with a dissociation constant (K_{D}) of less than about 100 pM, less than about 70 pM, less than about 50 pM, less than about 25 pM, less than about 20 pM, less than about 15 pM, or less than about 12 pM as measured in a surface plasmon resonance assay;
(d) binds to Activin A protein Gly311-Ser426 of UniProtKB/Swiss-Prot accession number P08476 (SEQ ID NO: 43) at 25° C. with a dissociation constant (K_{D}) of less than about 250 pM, less than about 225 pM, less than about 100 pM, less than about 50 pM, less than about 25 pM, or less than about 20 pM as measured in a surface plasmon resonance assay;
(e) binds to Activin A protein Gly311-Ser426 of UniProtKB/Swiss-Prot accession number P08476 (SEQ ID NO: 43) at 37° C. with a dissociation constant (K_{D}) of less than about 1 nM, less than about 750 pM, less than about 500 pM, less than about 200 pM, less than about 100 pM, lessthan about 50 pM, or less than about 25 pM as measured in a surface plasmon resonance assay;
(f) inhibits activation of cells expressing SMAD 2/3 transcription factors by human Activin A with a IC₅₀ of less than 5 nM, less than 3 nM, less than 2 nM, less than 1 nM, less than 0.5 nM, less than 0.25 nM, or less than 0.2 nM as measured in a cell-based bioassay;
(g) inhibits activation of cells expressing SMAD 2/3 transcription factors by GDF8 with a IC₅₀ of less than 20 nM, less than 15 nM, less than 10 nM, less than 8 nM, or less than 7 nM as measured in a cell-based bioassay; and (h) comprises (i) an HCVR of an antibody that binds GDF8, wherein the HCVR comprises an amino acid sequence selected from the group consisting of sequences listed in Table 1, (ii) an HCVR of an antibody that binds Activin A, wherein the HCVR comprises an amino acid sequence selected from the group consisting of sequences listed in Table 1; and (iii) an LCVR of an antibody that binds GDF8, wherein the LCVR comprises an amino acid sequence selected from the group consisting of sequences listed in Table 1. In some cases, the isolated bispecific antibody or antigen-binding fragment thereof does not include an LCVR of an antibody that binds Activin A. In some cases, the isolated bispecific antibody or antigen-binding fragment thereof does not include an LCVR comprising an amino acid sequence selected from the group consisting of LCVR sequences listed in Table 13.

In a second aspect, the present invention provides nucleic acid molecules encoding anti-GDF8 and anti-Activin A antibodies or portions thereof. For example, the present invention provides nucleic acid molecules encoding any of the HCVR amino acid sequences listed in Table 1; in certain embodiments the nucleic acid molecule comprises a polynucleotide sequence selected from any of the HCVR nucleic acid sequences listed in Table 2, or a substantially similar sequence thereof having at least 90%, at least 95%, at least 98% or at least 99% sequence identity thereto.

The present invention also provides nucleic acid molecules encoding any of the LCVR amino acid sequences listed in Table 1; in certain embodiments the nucleic acid molecule comprises a polynucleotide sequence selected from any of the LCVR nucleic acid sequences listed in Table 2, or a substantially similar sequence thereof having at least 90%, at least 95%, at least 98% or at least 99% sequence identity thereto.

The present invention also provides nucleic acid molecules encoding any of the HCDR1 amino acid sequences listed in Table 1; in certain embodiments the nucleic acid molecule comprises a polynucleotide sequence selected from any of the HCDR1 nucleic acid sequences listed in Table 2, or a substantially similar sequence thereof having at least 90%, at least 95%, at least 98% or at least 99% sequence identity thereto.

The present invention also provides nucleic acid molecules encoding any of the HCDR2 amino acid sequences listed in Table 1; in certain embodiments the nucleic acid molecule comprises a polynucleotide sequence selected from any of the HCDR2 nucleic acid sequences listed in Table 2, or a substantially similar sequence thereof having at least 90%, at least 95%, at least 98% or at least 99% sequence identity thereto.

The present invention also provides nucleic acid molecules encoding any of the HCDR3 amino acid sequences listed in Table 1; in certain embodiments the nucleic acid molecule comprises a polynucleotide sequence selected from any of the HCDR3 nucleic acid sequences listed in Table 2, or a substantially similar sequence thereof having at least 90%, at least 95%, at least 98% or at least 99% sequence identity thereto.

The present invention also provides nucleic acid molecules encoding any of the LCDR1 amino acid sequences listed in Table 1; in certain embodiments the nucleic acid molecule comprises a polynucleotide sequence selected from any of the LCDR1 nucleic acid sequences listed in Table 2, or a substantially similar sequence thereof having at least 90%, at least 95%, at least 98% or at least 99% sequence identity thereto.

The present invention also provides nucleic acid molecules encoding any of the LCDR2 amino acid sequences listed in Table 1; in certain embodiments the nucleic acid molecule comprises a polynucleotide sequence selected from any of the LCDR2 nucleic acid sequences listed in Table 2, or a substantially similar sequence thereof having at least 90%, at least 95%, at least 98% or at least 99% sequence identity thereto.

The present invention also provides nucleic acid molecules encoding any of the LCDR3 amino acid sequences listed in Table 1; in certain embodiments the nucleic acid molecule comprises a polynucleotide sequence selected from any of the LCDR3 nucleic acid sequences listed in Table 2, or a substantially similar sequence thereof having at least 90%, at least 95%, at least 98% or at least 99% sequence identity thereto.

The present invention also provides nucleic acid molecules encoding an HCVR, wherein the HCVR comprises a set of three CDRs (i.e., HCDR1-HCDR2-HCDR3), wherein the HCDR1-HCDR2-HCDR3 amino acid sequence set is as defined by any of the exemplary antibodies listed in Table 1.

The present invention also provides nucleic acid molecules encoding an LCVR, wherein the LCVR comprises a set of three CDRs (i.e., LCDR1-LCDR2-LCDR3), wherein the LCDR1-LCDR2-LCDR3 amino acid sequence set is as defined by any of the exemplary antibodies listed in Table 1.

The present invention also provides nucleic acid molecules encoding both an HCVR and an LCVR, wherein the HCVR comprises an amino acid sequence of any of the HCVR amino acid sequences listed in Table 1, and wherein the LCVR comprises an amino acid sequence of any of the LCVR amino acid sequences listed in Table 1. In certain embodiments, the nucleic acid molecule comprises a polynucleotide sequence selected from any of the HCVR nucleic acid sequences listed in Table 2, or a substantially similar sequence thereof having at least 90%, at least 95%, at least 98% or at least 99% sequence identity thereto, and a polynucleotide sequence selected from any of the LCVR nucleic acid sequences listed in Table 1, or a substantially similar sequence thereof having at least 90%, at least 95%, at least 98% or at least 99% sequence identity thereto. In certain embodiments according to this aspect of the invention, the nucleic acid molecule encodes an HCVR and LCVR, wherein the HCVR and LCVR are both derived from the same anti-GDF8 and anti-Activin A domains listed in Table 1.

In a related aspect, the present invention provides recombinant expression vectors capable of expressing a polypeptide comprising a heavy and/or light chain variable region of an antibody. For example, the present invention includes recombinant expression vectors comprising any of the nucleic acid molecules mentioned above, i.e., nucleic acid molecules encoding any of the HCVR, LCVR, and/or CDR sequences as set forth in Table 2.

In certain embodiments, the present invention provides expression vectors comprising: (a) a nucleic acid molecule comprising a nucleic acid sequence encoding a HCVR of an antibody that binds GDF8, wherein the HCVR comprises an amino acid sequence selected from the group consisting of sequences listed in Table 1, and (b) a nucleic acid molecule comprising a nucleic acid sequence encoding an HCVR of an antibody that binds Activin A, wherein the HCVR comprises an amino acid sequence selected from the group consisting of sequences listed in Table 1; and/or (c) a nucleic acid molecule comprising a nucleic acid sequence encoding a LCVR of an antibody that binds GDF8, wherein the LCVR comprises an amino acid sequence selected from the group consisting of sequences listed in Table 1.

Also included within the scope of the present invention are host cells into which such vectors have been introduced, as well as methods of producing the antibodies or portions thereof by culturing the host cells under conditions permitting production of the antibodies or antibody fragments, and recovering the antibodies and antibody fragments so produced. In certain embodiments, the host cells comprise a mammalian cell or a prokaryotic cell. In certain embodiments, the host cell is a Chinese Hamster Ovary (CHO) cell or an Escherichia coli (E. coli) cell. In certain embodiments, the present invention provides methods of producing an antibody or antigen-binding fragment thereof of the invention, the methods comprising introducing into a host cell an expression vector comprising a nucleic acid sequence encoding a HCVR and/or LCVR of an antibody or antigen-binding fragment thereof of the invention operably linked to a promoter; culturing the host cell under conditions favorable for expression of the nucleic acid sequence; and isolating the antibody or antigen-binding fragment thereof from the culture medium and/or host cell. The isolated antibody or antigen-binding fragment thereof may be purified using any of the methods known in prior art.

In a third aspect, the invention provides a pharmaceutical composition comprising a therapeutically effective amount of at least one recombinant monoclonal bispecific antibody or antigen-binding fragment thereof which specifically binds GDF8 and specifically binds Activin A and a pharmaceutically acceptable carrier. In a related aspect, the invention features a composition which is a combination of a bispecific antibody of the invention and a second therapeutic agent. In one embodiment, the second therapeutic agent is any agent that is advantageously combined with an GDF8 x Activin A bispecific antibody.

Exemplary agents that may be advantageously combined with the GDF8 x Activin A bispecific antibody include, without limitation, other agents that bind and/or inhibit GDF8 and/or Activin A activity (including other antibodies or antigen-binding fragments thereof, etc.) and/or agents which do not directly bind GDF8 or Activin A but nonetheless treat or ameliorate at least one symptom or indication of a GDF8-associated disease or disorder and/or Activin A-associated disease or disorder (disclosed elsewhere herein). Additional combination therapies and co-formulations involving the bispecific antibodies of the present invention are disclosed elsewhere herein.

In a fourth aspect, the invention provides therapeutic methods for treating a disease or disorder associated with GDF8 and/or Activin A in a subject using a GDF8 x Activin A bispecific antibody or antigen-binding portion of an antibody of the invention, wherein the therapeutic methods comprise administering a therapeutically effective amount of a pharmaceutical composition comprising an antibody or antigen-binding fragment of an antibody of the invention to the subject in need thereof.

The disorder treated is any disease or condition which is improved, ameliorated, inhibited or prevented by inhibiting GDF8 and/or Activin A activity. In certain embodiments, the invention provides methods to prevent, or treat a GDF8-associated and/or Activin A-associated disease or disorder comprising administering a therapeutically effective amount of an GDF8 x Activin A bispecific antibody or antigen-binding fragment thereof of the invention to a subject in need thereof.

In some embodiments, the antibody or antigen-binding fragment thereof may be administered prophylactically or therapeutically to a subject having or at risk of having a GDF8-associated and/or Activin A-associated disease or disorder. In certain embodiments, the antibody or antigen-binding fragment thereof the invention is administered in combination with a second therapeutic agent to the subject in need thereof.

The second therapeutic agent may be selected from the group consisting of an anti-Activin A antibody, or an antigen-binding fragment thereof, an anti-GDF8 antibody, or an antigen-binding fragment thereof, a growth factor inhibitor, an immunosuppressant, an anti-inflammatory agent, a metabolic inhibitor, a glucagon-like peptide-1 receptor agonist (GLP-1 agonist), an enzyme inhibitor, a cytotoxic/cytostatic agents, a lifestyle modification, a dietary supplement and any other drug or therapy known in the art. In certain embodiments, the second therapeutic agent may be an agent that helps to counteract or reduce any possible side effect(s) associated with an antibody or antigen-binding fragment thereof of the invention, if such side effect(s) should occur.

The present invention also includes use of a bispecific antigen-binding protein comprising an Activin A-specific binding domain and a GDF8-specific binding domain of the invention in the manufacture of a medicament for treating, preventing or ameliorating a disease or disorder characterized by decreased muscle mass or strength. In some cases, the disease or disorder characterized by decreased muscle mass or strength is selected from the group consisting of sarcopenia, cachexia, muscle injury, ligament injury, muscle wasting/atrophy, cancer, obesity, diabetes, arthritis, multiple sclerosis, muscular dystrophy, amyotrophic lateral sclerosis, Parkinson's disease, osteoporosis, osteoarthritis, osteopenia, and a metabolic syndrome.

The present invention also includes use of a bispecific antigen-binding protein comprising an Activin A-specific binding domain and a GDF8-specific binding domain of the invention in the manufacture of a medicament for increasing muscle mass or strength in a subject.

The bispecific antibody or fragment thereof may be administered subcutaneously, intravenously, intradermally, intraperitoneally, orally, intramuscularly, or intracerebroventricularly.

The antibody or fragment thereof may be administered at a dose of about 0.1 mg/kg of body weight to about 100 mg/kg of body weight of the subject. In certain embodiments, an antibody of the present invention may be administered at one or more doses comprising between 10 mg to 600 mg.

Other embodiments will become apparent from a review of the ensuing detailed description.

### BRIEF DESCRIPTION OF THE FIGURES

FIG. 1A shows a graph of effects on mean body weights in mice over time as change from baseline in g of REGN1033 and REGN2477 compared to H4H11419D bispecific antibody in obese, male DIO mice.

FIG. 1B shows a graph of effects on final mean body weights in mice as change from baseline in g of REGN1033 and REGN2477 compared to H4H11419D bispecific antibody in obese, male DIO mice.

FIG. 2A shows a graph of effects on mean lean mass weights in mice over time as change from baseline in g of REGN1033 and REGN2477 compared to H4H11419D bispecific antibody in obese, male DIO mice.

FIG. 2B shows a bar graph of effects on final mean lean mass weights in mice as change from baseline in g of REGN1033 and REGN2477 compared to H4H11419D bispecific antibody in obese, male DIO mice.

FIG. 2C shows a graph of effects on mean fat mass weights in mice over time as change from baseline in g of REGN1033 and REGN2477 compared to H4H11419D bispecific antibody in obese, male DIO mice.

FIG. 2D shows a bar graph of effects on mean final fat mass weights in mice as change from baseline in g of REGN1033 and REGN2477 compared to H4H11419D bispecific antibody in obese, male DIO mice.

FIG. 3A shows a bar graph of effects on mean final TA weights (mg) in mice of REGN1033 and REGN2477 compared to H4H11419D in obese, male DIO mice.

FIG. 3B shows a bar graph of effects on mean final GA/Sol weights (mg) in mice of REGN1033 and REGN2477 compared to H4H11419D in obese, male DIO mice.

FIG. 3C shows a bar graph of effects on mean final quad weights (mg) in mice of REGN1033 and REGN2477 compared to H4H11419D in obese, male DIO mice.

FIG. 3D shows a bar graph of effects on mean final scWAT weights (mg) in mice of REGN1033 and REGN2477 compared to H4H11419D in obese, male DIO mice.

FIG. 3E shows a bar graph of effects on mean final gWAT weights (mg) in mice of REGN1033 and REGN2477 compared to H4H11419D in obese, male DIO mice.

FIG. 3F shows a bar graph of effects on mean final liver weights (mg) in mice of REGN1033 and REGN2477 compared to H4H11419D in obese, male DIO mice.

FIG. 3G shows a bar graph of effects on mean final heart weights (mg) in mice of REGN1033 and REGN2477 compared to H4H11419D in obese, male DIO mice.

### DETAILED DESCRIPTION

Before the present methods are described, it is to be understood that this invention is not limited to particular methods, and experimental conditions described, as such methods and conditions may vary. It is also to be understood that the terminology used herein is for the purpose of describing particular embodiments only, and is not intended to be limiting, since the scope of the present invention will be limited only by the appended claims.

Unless defined otherwise, all technical and scientific terms used herein have the same meaning as commonly understood by one of ordinary skill in the art to which this invention belongs.

Although any methods and materials similar or equivalent to those described herein can be used in the practice or testing of the present invention, preferred methods and materials are now described. All publications, patents, and patent applications mentioned herein are incorporated herein by reference in their entirety.

### Definitions

As used herein, the term “about,” when used in reference to a particular recited numerical value, means that the value may vary from the recited value by no more than 1%. For example, as used herein, the expression “about 100” includes 99 and 101 and all values in between (e.g., 99.1, 99.2, 99.3, 99.4, etc.).

As used herein, the expression “antigen-binding molecule” means a protein, polypeptide or molecular complex comprising or consisting of at least one complementarity determining region (CDR) that alone, or in combination with one or more additional CDRs and/or framework regions (FRs), specifically binds to a particular antigen. In embodiments, an antigen binding molecule comprises one or more antigen binding domains. The term “antigen-binding molecule” includes antibodies and antigen-binding fragments of antibodies, including, e.g., bispecific antibodies. In some cases, the bispecific antibodies may comprise a common light chain.

The term “antibody”, as used herein, means any antigen-binding molecule or molecular complex comprising at least one complementarity determining region (CDR) that specifically binds to or interacts with a particular antigen (e.g., activin A or GDF8). The term “antibody” includes immunoglobulin molecules comprising four polypeptide chains, two heavy (H) chains and two light (L) chains inter-connected by disulfide bonds, as well as multimers thereof (e.g., IgM). Each heavy chain comprises a heavy chain variable region (abbreviated herein as HCVR or V_{H}) and a heavy chain constant region. The heavy chain constant region comprises three domains, C_{H}1, C_{H}2 and C_{H}3. Each light chain comprises a light chain variable region (abbreviated herein as LCVR or V_{L}) and a light chain constant region. The light chain constant region comprises one domain (C_{L}1). The V_{H} and V_{L} regions can be further subdivided into regions of hypervariability, termed complementarity determining regions (CDRs), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. In different embodiments of the disclosure, the FRs of the anti-C1q antibody (or antigen-binding portion thereof) may be identical to the human germline sequences, or may be naturally or artificially modified. In different embodiments of the disclosure, the FRs of the anti-IsdB antibody (or antigen-binding portion thereof) may be identical to the human germline sequences, or may be naturally or artificially modified. An amino acid consensus sequence may be defined based on a side-by-side analysis of two or more CDRs.

The term “antibody”, as used herein, also includes antigen-binding fragments of full antibody molecules. The terms “antigen-binding portion” of an antibody, “antigen binding domain” of an antibody, “antigen-binding fragment” of an antibody, and the like, as used herein, include any naturally occurring, enzymatically obtainable, synthetic, or genetically engineered polypeptide or glycoprotein that specifically binds an antigen to form a complex. Antigen-binding fragments of an antibody may be derived, e.g., from full antibody molecules using any suitable standard techniques such as proteolytic digestion or recombinant genetic engineering techniques involving the manipulation and expression of DNA encoding antibody variable and optionally constant domains. Such DNA is known and/or is readily available from, e.g., commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

As used herein, the expression “antigen-binding domain” means any peptide, polypeptide, nucleic acid molecule, scaffold-type molecule, peptide display molecule, or polypeptide-containing construct that is capable of specifically binding a particular antigen of interest (e.g., Activin A or GDF8).

Non-limiting examples of antigen-binding fragments include: (i) Fab fragments; (ii) F(ab′)2 fragments; (iii) Fd fragments; (iv) Fv fragments; (v) single-chain Fv (scFv) molecules; (vi) dAb fragments; (vii) an antigen binding domain, and (viii) minimal recognition units consisting of the amino acid residues that mimic the hypervariable region of an antibody (e.g., an isolated complementarity determining region (CDR) such as a CDR3 peptide), or a constrained FR3-CDR3-FR4 peptide. Other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g. monovalent nanobodies, bivalent nanobodies, etc.), small modular immunopharmaceuticals (SMIPs), and shark variable IgNAR domains, are also encompassed within the expression “antigen-binding fragment,” as used herein.

An antigen-binding fragment of an antibody will typically comprise at least one variable domain. The variable domain may be of any size or amino acid composition and will generally comprise at least one CDR which is adjacent to or in frame with one or more framework sequences. In antigen-binding fragments having a V_{H} domain associated with a V_{L} domain, the V_{H} and V_{L} domains may be situated relative to one another in any suitable arrangement. For example, the variable region may be dimeric and contain V_{H}-V_{H}, V_{H}-V_{L} or V_{L}-V_{L} dimers. Alternatively, the antigen-binding fragment of an antibody may contain a monomeric V_{H} or V_{L} domain.

In certain embodiments, an antigen-binding fragment of an antibody may contain at least one variable domain covalently linked to at least one constant domain. Non-limiting, exemplary configurations of variable and constant domains that may be found within an antigen-binding fragment of an antibody of the present disclosure include: (i) V_{H}-C_{H}1; (ii) V_{H}-C_{H}2; (iii) V_{H}-C_{H}3; (iv) V_{H}-C_{H}1-C_{H}2; (v) V_{H}-C_{H}1-C_{H}2-C_{H}3; (vi) V_{H}-C_{H}2-C_{H}3; (vii) V_{H}-C_{L}; (viii) V_{L}-C_{H}1; (ix) V_{L}-C_{H}2; (X) V_{L}-C_{H}3; (xi) V_{L}-C_{H}1-C_{H}2; (xii) V_{L}-C_{H}1-C_{H}2-C_{H}3; (xiii) V_{L}-C_{H}2-C_{H}3; and (xiv) V_{L}-C_{L}. In any configuration of variable and constant domains, including any of the exemplary configurations listed above, the variable and constant domains may be either directly linked to one another or may be linked by a full or partial hinge or linker region. A hinge region may comprise or consist of at least 2 (e.g., 5, 10, 15, 20, 40, 60 or more) amino acids which result in a flexible or semi-flexible linkage between adjacent variable and/or constant domains in a single polypeptide molecule. Moreover, an antigen-binding fragment of an antibody of the present disclosure may comprise a homo-dimer or hetero-dimer (or other multimer) of any of the variable and constant domain configurations listed above in non-covalent association with one another and/or with one or more monomeric V_{H} or V_{L} domain (e.g., by disulfide bond(s)).

As with full antibody molecules, antigen-binding fragments may be monospecific or multispecific (e.g., bispecific). A multispecific antigen-binding fragment of an antibody will typically comprise at least two different variable domains, wherein each variable domain is capable of specifically binding to a separate antigen or to a different epitope on the same antigen. Any multispecific antibody format, including the exemplary bispecific antibody formats disclosed herein, may be adapted for use in the context of an antigen-binding fragment of an antibody of the present disclosure using routine techniques available in the art. In some cases, the bispecific antibodies of the disclosure comprise a common light chain.

The term “force pairing” of a common light chain in a bispecific antibody refers to wherein no engineering is applied to force pairing of the chains. In some cases, both monospecific and bispecific antibodies may be secreted in the supernatant. In this context, stoichiometric expression of the two light chains allows for maximum assembly.

The term “specifically binds” or the like, as used herein, means that the antigen-binding domain forms a complex with a particular antigen characterized by a dissociation constant (K_{D}) of 25 nM or less. Exemplary categories of antigen-binding domains that can be used in the context of the present disclosure include antibodies, antigen-binding portions of antibodies, peptides that specifically interact with a particular antigen (e.g., peptibodies), receptor molecules that specifically interact with a particular antigen, proteins comprising a ligand-binding portion of a receptor that specifically binds a particular antigen, antigen-binding scaffolds (e.g., DARPins, HEAT repeat proteins, ARM repeat proteins, tetratricopeptide repeat proteins, and other scaffolds based on naturally occurring repeat proteins, etc., [see, e.g., Boersma and Pluckthun, 2011, Curr. Opin. Biotechnol. 22:849-857, and references cited therein]), and aptamers or portions thereof.

Methods for determining whether two molecules specifically bind one another are well known in the art and include, for example, equilibrium dialysis, surface plasmon resonance, and the like. For example, an antigen-binding molecule, as used in the context of the present disclosure, includes polypeptides that bind a particular antigen (e.g., a target molecule [T] or a portion thereof) with a K_{D} of less than about 25 nM, less than about 20 nM, less than about 15 nM, less than about 10 nM, less than about 5 nM, less than about 1 nM, less than about 500 pM, less than about 400 pM, less than about 300 pM, less than about 200 pM, less than about 100 pM, less than about 90 pM, less than about 80 pM, less than about 70 pM, less than about 60 pM, less than about 50 pM, less than about 40 pM, less than about 30 pM, less than about 20 pM, less than about 10 pM, less than about 5 pM, less than about 4 pM, less than about 2 pM, less than about 1 pM, less than about 0.5 pM, less than about 0.2 pM, less than about 0.1 pM, or less than about 0.05 pM, as measured in a surface plasmon resonance assay.

The term “SEQ ID NO: 21” refers to the DNA sequence ACT ACA TCC. The term “SEQ ID NO: 22” refers to the amino acid sequence Thr Thr Ser. The term “SEQ ID NO: 46” refers to the amino acid sequence Gly Ala Ser. The term “SEQ ID NO: 54” refers to the amino acid sequence SEQ ID NO: Asp Ala Ser. The term “SEQ ID NO: 62” refers to the amino acid sequence Ala Ala Ser.

### Antigen-Specific Binding Proteins

The present invention relates to compositions comprising bispecific antigen-binding proteins. More specifically, the present invention provides a bispecific antigen-binding molecule comprising an Activin A-specific binding domain and a GDF8-specific binding domain.

As used herein, the expression “antigen-specific binding protein” means a protein comprising at least one domain which specifically binds a particular antigen. Exemplary categories of antigen-specific binding proteins include antibodies, antigen-binding portions of antibodies, peptides that specifically interact with a particular antigen (e.g., peptibodies), receptor molecules that specifically interact with a particular antigen, and proteins comprising a ligand-binding portion of a receptor that specifically binds a particular antigen.

Antigen-Binding Molecules with Two Different Antigen-Specific Binding Domains

The present invention also includes antigen-binding molecules comprising two different antigen-specific binding domains. In particular, the present invention includes antigen-binding molecules comprising an Activin A-specific binding domain and a GDF8-specific binding domain. The term “antigen-specific binding domain,” as used herein, includes polypeptides comprising or consisting of: (i) an antigen-binding fragment of an antibody molecule, (ii) a peptide that specifically interacts with a particular antigen (e.g., a peptibody), and/or (iii) a ligand-binding portion of a receptor that specifically binds a particular antigen. In some cases, the bispecific antibodies may be IgG type antibodies having two different heavy chain variable regions.

For example, the present invention includes bispecific antibodies with one arm comprising a first heavy chain variable region/light chain variable region (HCVR/LCVR) pair that specifically binds Activin A and another arm comprising a second HCVR/LCVR pair that specifically binds GDF8. The bispecific antibodies may be produced in any format known in the art. In some cases, the bispecific antibodies comprise a common light chain LCVR. In some cases, the method of making the bispecific antibodies may comprise force pairing of a common light chain in the bispecific antibody. In some cases, the common light chain is a GDF8 common light chain. In some cases, the common light chain is an Activin A common light chain.

The present invention includes bi-specific antigen-binding proteins comprising a first antigen-binding domain (D1) that specifically binds GDF8, i.e., “GDF8-specific binding domain,” and a second antigen-binding domain (D2) that specifically binds Activin A, i.e., “Activin A-specific binding domain.” Activins are homo- and hetero-dimeric molecules comprising beta subunits, i.e., Inhibin RA, inhibin RB, inhibin RC, and/or inhibin RE. The @A subunit has the amino acid sequence of SEQ ID NO: 40 and the @3B subunit has the amino acid sequence of SEQ ID NO: 41. Activin A is a homodimer of two sA subunits; Activin B is a homodimer of two @B subunits; Activin AB is a heterodimer of one @A subunit and one @B subunit; and Activin AC is a heterodimer of one @A subunit and one pC subunit. An Activin A-specific binding protein may be an antigen-specific binding protein that specifically binds the @A subunit. Since the @A subunit is found in Activin A, Activin AB, and Activin AC molecules, an “Activin A-specific binding domain” can be an antigen-specific binding protein that specifically binds Activin A as well as Activin AB and Activin AC (by virtue of its interaction with the @A subunit). Therefore, according to one embodiment of the present invention, an Activin A-specific binding domain specifically binds Activin A; or Activin A and Activin AB; or Activin A and Activin AC; or Activin A, Activin AB and Activin AC, but does not bind other ActRIIB ligands such as Activin B, GDF3, BMP2, BMP4, BMP7, BMP9, BMP10, GDF11, Nodal, etc. Thus, in one embodiment of the invention, an Activin A-specific binding domain specifically binds to Activin A but does not bind significantly to Activin B or Activin C. In another embodiment, an Activin A-specific binding domain may also bind to Activin B (by virtue of cross-reaction with the βB subunit, i.e., InhibinβB). In another embodiment, an Activin A-specific binding domain is a binding protein that binds specifically to Activin A but does not bind to any other ligand of ActRIIB. In another embodiment, an Activin A-specific binding domain is a binding protein and binds specifically to Activin A and does not bind to any Bone Morphogenetic Protein (BMP) (e.g., BMP2, BMP4, BMP6, BMP9, BMP10). In another embodiment, an Activin A-specific binding domain is a binding protein that binds specifically to Activin A but does not bind to any other member of the transforming growth factor beta (TGFβ) superfamily.

The present invention includes bi-specific antigen-binding proteins comprising a second antigen-binding domain (D2) that specifically binds Activin A, i.e., “Activin A-specific binding domain” and a second antigen-binding domain (D2) that specifically binds GDF8, i.e., “GDF8-specific binding domain.” The term “GDF8” (also referred to as “growth and differentiation factor-8” and “myostatin”) means the protein comprising the amino acid sequence of SEQ ID NO: 42 (mature protein). According to the present invention, GDF8-specific binding domains specifically bind GDF8 but do not bind other ActRIIB ligands such as GDF3, BMP2, BMP4, BMP7, BMP9, BMP10, GDF11, Nodal, etc.

In the context of the present invention, molecules such as ActRIIB-Fc (e.g., “ACE-031”), which comprise the ligand-binding portion of the ActRIIB receptor, are not considered “Activin A-specific binding proteins” or “GDF8-specific binding proteins” because such molecules bind multiple ligands besides GDF8, Activin A and Activin AB.

All references to proteins, polypeptides and protein fragments herein are intended to refer to the human version of the respective protein, polypeptide or protein fragment unless explicitly specified as being from a non-human species.

The term “antibody”, as used herein, is intended to refer to immunoglobulin molecules comprised of four polypeptide chains, two heavy (H) chains and two light (L) chains inter-connected by disulfide bonds (i.e., “full antibody molecules”), as well as multimers thereof (e.g. IgM) or antigen-binding fragments thereof. Each heavy chain is comprised of a heavy chain variable region (“HCVR” or “V_{H}”) and a heavy chain constant region (comprised of domains C_{H}1, C_{H}2 and C_{H}3). Each light chain is comprised of a light chain variable region (“LCVR or “V_{L}”) and a light chain constant region (C_{L}). The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. In certain embodiments of the invention, the FRs of the antibody (or antigen binding fragment thereof) may be identical to the human germline sequences, or may be naturally or artificially modified. An amino acid consensus sequence may be defined based on a side-by-side analysis of two or more CDRs.

Substitution of one or more CDR residues or omission of one or more CDRs is also possible. Antibodies have been described in the scientific literature in which one or two CDRs can be dispensed with for binding. Padlan et al. (1995 FASEB J. 9:133-139) analyzed the contact regions between antibodies and their antigens, based on published crystal structures, and concluded that only about one fifth to one third of CDR residues actually contact the antigen. Padlan also found many antibodies in which one or two CDRs had no amino acids in contact with an antigen (see also, Vajdos et al. 2002 J Mol Biol 320:415-428).

CDR residues not contacting antigen can be identified based on previous studies (for example residues H60-H65 in CDRH2 are often not required), from regions of Kabat CDRs lying outside Chothia CDRs, by molecular modeling and/or empirically. If a CDR or residue(s) thereof is omitted, it is usually substituted with an amino acid occupying the corresponding position in another human antibody sequence or a consensus of such sequences. Positions for substitution within CDRs and amino acids to substitute can also be selected empirically. Empirical substitutions can be conservative or non-conservative substitutions.

The fully human antigen-binding molecules antibodies disclosed herein may comprise one or more amino acid substitutions, insertions and/or deletions in the framework and/or CDR regions of the heavy and light chain variable domains as compared to the corresponding germline sequences. Such mutations can be readily ascertained by comparing the amino acid sequences disclosed herein to germline sequences available from, for example, public antibody sequence databases. The present invention includes antibodies, and antigen-binding fragments thereof, which are derived from any of the amino acid sequences disclosed herein, wherein one or more amino acids within one or more framework and/or CDR regions are mutated to the corresponding residue(s) of the germline sequence from which the antibody was derived, or to the corresponding residue(s) of another human germline sequence, or to a conservative amino acid substitution of the corresponding germline residue(s) (such sequence changes are referred to herein collectively as “germline mutations”). A person of ordinary skill in the art, starting with the heavy and light chain variable region sequences disclosed herein, can easily produce numerous antibodies and antigen-binding fragments which comprise one or more individual germline mutations or combinations thereof. In certain embodiments, all of the framework and/or CDR residues within the V_{H} and/or V_{L} domains are mutated back to the residues found in the original germline sequence from which the antibody was derived. In other embodiments, only certain residues are mutated back to the original germline sequence, e.g., only the mutated residues found within the first 8 amino acids of FR1 or within the last 8 amino acids of FR4, or only the mutated residues found within CDR1, CDR2 or CDR3. In other embodiments, one or more of the framework and/or CDR residue(s) are mutated to the corresponding residue(s) of a different germline sequence (i.e., a germline sequence that is different from the germline sequence from which the antibody was originally derived). Furthermore, the antibodies of the present invention may contain any combination of two or more germline mutations within the framework and/or CDR regions, e.g., wherein certain individual residues are mutated to the corresponding residue of a particular germline sequence while certain other residues that differ from the original germline sequence are maintained or are mutated to the corresponding residue of a different germline sequence. Once obtained, antibodies and antigen-binding fragments that contain one or more germline mutations can be easily tested for one or more desired property such as, improved binding specificity, increased binding affinity, improved or enhanced antagonistic biological properties, reduced immunogenicity, etc. Antibodies and antigen-binding fragments obtained in this general manner are encompassed within the present invention.

The present invention also includes fully human antigen-binding molecules comprising variants of any of the HCVR, LCVR, and/or CDR amino acid sequences disclosed herein having one or more conservative substitutions. For example, the present invention includes antigen-binding molecules having HCVR, LCVR, and/or CDR amino acid sequences with, e.g., 10 or fewer, 8 or fewer, 6 or fewer, 4 or fewer, etc. conservative amino acid substitutions relative to any of the HCVR, LCVR, and/or CDR amino acid sequences disclosed herein.

The term “human antigen-binding molecule”, “human antibody”, or “fully human antibody”, as used herein, is intended to include antibodies having variable and constant regions derived from human germline immunoglobulin sequences. The human mAbs of the invention may include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antigen-binding molecule”, “human antibody”, or “fully human antibody”, as used herein, is not intended to include mAbs in which CDR sequences derived from the germline of another mammalian species (e.g., mouse), have been grafted onto human FR sequences. The term includes antibodies and antigen-binding molecules that are recombinantly produced in a non-human mammal, or in cells of a non-human mammal. The term is not intended to include antibodies isolated from or generated in a human subject.

The term “recombinant”, as used herein, refers to antigen-binding molecules, antibodies or antigen-binding fragments thereof of the invention created, expressed, isolated or obtained by technologies or methods known in the art as recombinant DNA technology which include, e.g., DNA splicing and transgenic expression. The term refers to antibodies expressed in a non-human mammal (including transgenic non-human mammals, e.g., transgenic mice), or a cell (e.g., CHO cells) expression system or isolated from a recombinant combinatorial human antibody library.

### Specific Binding

The term “specifically binds,” or “binds specifically to”, or the like, means that an antigen-binding molecule, such as an antibody or antigen-binding fragment thereof forms a complex with an antigen that is relatively stable under physiologic conditions. Specific binding can be characterized by an equilibrium dissociation constant of at least about 1×10⁻⁸ M or less (e.g., a smaller K_{D} denotes a tighter binding). Methods for determining whether two molecules specifically bind are well known in the art and include, for example, equilibrium dialysis, surface plasmon resonance, and the like. As described herein, antibodies have been identified by surface plasmon resonance, e.g., BIACORE™, which bind specifically to Activin A, Activin AB, and GDF8. Moreover, multi-specific antibodies that bind to one domain in Activin A and a second domain in GDF8 or a bi-specific that binds to different regions of Activin A or GDF8 are nonetheless considered antibodies that “specifically bind”, as used herein.

The term “high affinity” antibody refers to those mAbs having a binding affinity to Activin A and/or GDF8, expressed as K_{D}, of at least 10⁻⁸ M; preferably 10⁻⁹ M; more preferably 10⁻¹⁰M, even more preferably 10⁻¹¹ M, as measured by surface plasmon resonance, e.g., BIACORE™ or solution-affinity ELISA.

In some cases, the term “specifically binds” or the like, or “binds specifically to”, or the like, as used herein, means that an antigen-specific binding protein, or an antigen-specific binding domain, forms a complex with a particular antigen characterized by a dissociation constant (K_{D}) of 500 pM or less, and does not bind other unrelated antigens under ordinary test conditions. “Unrelated antigens” are proteins, peptides or polypeptides that have less than 95% amino acid identity to one another. Methods for determining whether two molecules specifically bind one another are well known in the art and include, for example, equilibrium dialysis, surface plasmon resonance, and the like. For example, an antigen-specific binding protein or an antigen-specific binding domain, as used in the context of the present invention, includes molecules that bind a particular antigen (e.g., Activin A and/or AB, or GDF8) or a portion thereof with a K_{D} of less than about 500 pM, less than about 400 pM, less than about 300 pM, less than about 200 pM, less than about 100 pM, less than about 90 pM, less than about 80 pM, less than about 70 pM, less than about 60 pM, less than about 50 pM, less than about 40 pM, less than about 30 pM, less than about 20 pM, less than about 10 pM, less than about 5 pM, less than about 4 pM, less than about 2 pM, less than about 1 pM, less than about 0.5 pM, less than about 0.2 pM, less than about 0.1 pM, or less than about 0.05 pM, as measured in a surface plasmon resonance assay.

By the term “slow off rate”, “Koff” or “kd” is meant an antibody that dissociates from an antigen such as Activin A or GDF8, with a rate constant of 1×10⁻³ s⁻¹ or less, preferably 1×10⁻⁴ s⁻¹ or less, as determined by surface plasmon resonance, e.g., BIACORE™.

As used herein, an antigen-specific binding protein or antigen-specific binding domain “does not bind” to a specified molecule (e.g., “does not bind GDF11”, “does not bind BMP9”, “does not bind BMP10”, etc.) if the protein or binding domain, when tested for binding to the molecule at 25° C. in a surface plasmon resonance assay, exhibits a K_{D} of greater than 50.0 nM, or fails to exhibit any binding in such an assay or equivalent thereof.

The term “surface plasmon resonance”, as used herein, refers to an optical phenomenon that allows for the analysis of real-time interactions by detection of alterations in protein concentrations within a biosensor matrix, for example using the BIAcore™ system (Biacore Life Sciences division of GE Healthcare, Piscataway, NJ).

The term “K_{D}”, as used herein, means the equilibrium dissociation constant of a particular protein-protein interaction (e.g., antibody-antigen interaction). Unless indicated otherwise, the K_{D} values disclosed herein refer to K_{D} values determined by surface plasmon resonance assay at 25° C.

The terms “antigen-binding portion” of an antibody, “antigen-binding fragment” of an antigen-binding molecule, such as an antibody, and the like, as used herein, include any naturally occurring, enzymatically obtainable, synthetic, or genetically engineered polypeptide or glycoprotein that specifically binds an antigen to form a complex. The terms “antigen-binding fragment” of an antibody, or “antibody fragment”, as used herein, refers to one or more fragments of an antibody that retain the ability to bind to Activin A and/or GDF8 proteins.

In specific embodiments, antigen-binding molecules, antibody or antibody fragments of the invention may be conjugated to a moiety such a ligand or a therapeutic moiety (“immunoconjugate”), a second anti-Activin A or anti-GDF8 antibody, or any other therapeutic moiety useful for treating a Activin A-associated or GDF8-associated disease or disorder.

An “isolated antigen-binding molecule” or “isolated antibody”, as used herein, is intended to refer to an antigen-binding molecule or antibody that is substantially free of other antigen-binding molecules or antibodies (Abs) having different antigenic specificities. An isolated antigen-binding molecule comprising a first antigen-binding domain that specifically binds GDF8 and a second antigen-binding domain that specifically binds Activin A, is substantially free of binding domains that specifically bind antigens other than Activin A or GDF8.

An “deactivating antibody” or an “antagonist antibody”, as used herein (or an “antibody that decreases or blocks Activin A activity”, “antibody that decreases or blocks GDF8 activity” or “an antibody that destabilizes the activated conformation”), is intended to refer to an antibody whose binding to Activin A and/or GDF8 results in deactivation of at least one biological activity of Activin A and/or GDF8. For example, an antibody of the invention may decrease cachexia, sarcopenia, and other muscle-wasting conditions upon administration to a subject in need thereof.

The term “epitope” refers to an antigenic determinant that interacts with a specific antigen binding site in the variable region of an antibody or antigen-binding molecule known as a paratope.

A single antigen may have more than one epitope. Thus, different antibodies may bind to different areas on an antigen and may have different biological effects. The term “epitope” also refers to a site on an antigen to which B and/or T cells respond. It also refers to a region of an antigen that is bound by an antibody. B-cell epitopes can be formed both from contiguous amino acids or noncontiguous amino acids juxtaposed by tertiary folding of a protein. Epitopes formed from contiguous amino acids are typically retained on exposure to denaturing solvents, whereas epitopes formed by tertiary folding are typically lost on treatment with denaturing solvents. An epitope typically includes at least 3, and more usually, at least 5 or 8-10 amino acids in a unique spatial conformation. Epitopes may be defined as structural or functional. Functional epitopes are generally a subset of the structural epitopes and have those residues that directly contribute to the affinity of the interaction. Epitopes may also be conformational, that is, composed of non-linear amino acids. In certain embodiments, epitopes may include determinants that are chemically active surface groupings of molecules such as amino acids, sugar side chains, phosphoryl groups, or sulfonyl groups, and, in certain embodiments, may have specific three-dimensional structural characteristics, and/or specific charge characteristics.

The term “cross-competes”, as used herein, means an antigen-binding molecule, antibody or antigen-binding fragment thereof binds to an antigen and inhibits or blocks the binding of another antibody or antigen-binding fragment thereof. The term also includes competition between two antibodies in both orientations, i.e., a first antibody that binds and blocks binding of second antibody and vice-versa. In certain embodiments, the first antibody and second antibody may bind to the same epitope. Alternatively, the first and second antibodies may bind to different, but overlapping epitopes such that binding of one inhibits or blocks the binding of the second antibody, e.g., via steric hindrance. Cross-competition between antibodies may be measured by methods known in the art, for example, by a real-time, label-free bio-layer interferometry assay. Cross-competition between two antibodies may be expressed as the binding of the second antibody that is less than the background signal due to self-self binding (wherein first and second antibodies is the same antibody). Cross-competition between 2 antibodies may be expressed, for example, as % binding of the second antibody that is less than the baseline self-self background binding (wherein first and second antibodies is the same antibody).

The term “substantial identity” or “substantially identical,” when referring to a nucleic acid or fragment thereof, indicates that, when optimally aligned with appropriate nucleotide insertions or deletions with another nucleic acid (or its complementary strand), there is nucleotide sequence identity in at least about 90%, and more preferably at least about 95%, 96%, 97%, 98% or 99% of the nucleotide bases, as measured by any well-known algorithm of sequence identity, such as FASTA, BLAST or GAP, as discussed below. A nucleic acid molecule having substantial identity to a reference nucleic acid molecule may, in certain instances, encode a polypeptide having the same or substantially similar amino acid sequence as the polypeptide encoded by the reference nucleic acid molecule.

As applied to polypeptides, the term “substantial similarity” or “substantially similar” means that two peptide sequences, when optimally aligned, such as by the programs GAP or BESTFIT using default gap weights, share at least 90% sequence identity, even more preferably at least 95%, 98% or 99% sequence identity. Preferably, residue positions, which are not identical, differ by conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent or degree of similarity may be adjusted upwards to correct for the conservative nature of the substitution. Means for making this adjustment are well known to those of skill in the art. See, e.g., Pearson (1994) Methods Mol. Biol. 24: 307-331, which is herein incorporated by reference. Examples of groups of amino acids that have side chains with similar chemical properties include 1) aliphatic side chains: glycine, alanine, valine, leucine and isoleucine; 2) aliphatic-hydroxyl side chains: serine and threonine; 3) amide-containing side chains: asparagine and glutamine; 4) aromatic side chains: phenylalanine, tyrosine, and tryptophan; 5) basic side chains: lysine, arginine, and histidine; 6) acidic side chains: aspartate and glutamate, and 7) sulfur-containing side chains: cysteine and methionine. Preferred conservative amino acids substitution groups are: valine-leucine-isoleucine, phenylalanine-tyrosine, lysine-arginine, alanine-valine, glutamate-aspartate, and asparagine-glutamine. Alternatively, a conservative replacement is any change having a positive value in the PAM250 log-likelihood matrix disclosed in Gonnet et al. (1992) Science 256: 1443 45, herein incorporated by reference. A “moderately conservative” replacement is any change having a nonnegative value in the PAM250 log-likelihood matrix.

Sequence similarity for polypeptides is typically measured using sequence analysis software. Protein analysis software matches similar sequences using measures of similarity assigned to various substitutions, deletions and other modifications, including conservative amino acid substitutions. For instance, GCG software contains programs such as GAP and BESTFIT which can be used with default parameters to determine sequence homology or sequence identity between closely related polypeptides, such as homologous polypeptides from different species of organisms or between a wild type protein and a mutein thereof. See, e.g., GCG Version 6.1. Polypeptide sequences also can be compared using FASTA with default or recommended parameters; a program in GCG Version 6.1. FASTA (e.g., FASTA2 and FASTA3) provides alignments and percent sequence identity of the regions of the best overlap between the query and search sequences (Pearson (2000) supra). Another preferred algorithm when comparing a sequence of the invention to a database containing a large number of sequences from different organisms is the computer program BLAST, especially BLASTP or TBLASTN, using default parameters. See, e.g., Altschul et al. (1990) J. Mol. Biol. 215: 403-410 and (1997) Nucleic Acids Res. 25:3389-3402, each of which is herein incorporated by reference.

By the phrase “therapeutically effective amount” is meant an amount that produces the desired effect for which it is administered. The exact amount will depend on the purpose of the treatment, and will be ascertainable by one skilled in the art using known techniques (see, for example, Lloyd (1999) The Art, Science and Technology of Pharmaceutical Compounding).

As used herein, the term “subject” refers to an animal, preferably a mammal, more preferably a human, in need of amelioration, prevention and/or treatment of a Activin A-associated disease or disorder or GDF8-associate disease or disorder such as cachexia, sarcopenia, and other muscle-wasting conditions. The term includes human subjects who have or are at risk of having such a disease or disorder.

As used herein, the terms “treat”, “treating”, or “treatment” refer to the reduction or amelioration of the severity of at least one symptom or indication of a Activin A-associated or GDF8-associated disease or disorder due to the administration of a therapeutic agent such as an antigen-binding molecule such as an antibody of the present invention to a subject in need thereof. The terms include inhibition of progression of disease or of worsening of a symptom/indication. The terms also include positive prognosis of disease, i.e., the subject may be free of disease or may have reduced disease upon administration of a therapeutic agent such as an antibody of the present invention. The therapeutic agent may be administered at a therapeutic dose to the subject.

The terms “prevent”, “preventing” or “prevention” refer to inhibition of manifestation of a Activin A-associated disease or disorder, a GDF8-associated disease or disorder, or any symptoms or indications of such a disease or disorder upon administration of a bispecific antigen binding molecule of the present invention.

### Antigen-Binding Fragments of Antibodies

Unless specifically indicated otherwise, the term “antibody,” as used herein, shall be understood to encompass antibody molecules comprising two immunoglobulin heavy chains and two immunoglobulin light chains (i.e., “full antibody molecules”) as well as antigen-binding fragments thereof. The terms “antigen-binding portion” of an antibody, “antigen-binding fragment” of an antibody, and the like, as used herein, include any naturally occurring, enzymatically obtainable, synthetic, or genetically engineered polypeptide or glycoprotein that specifically binds an antigen to form a complex. The terms “antigen-binding fragment” of an antibody, or “antibody fragment”, as used herein, refers to one or more fragments of an antibody that retain the ability to specifically bind to an GDF8 protein, an Activin A protein, a fragment thereof, and/or mutant thereof. An antibody fragment may include a Fab fragment, a F(ab′)₂ fragment, a Fv fragment, a dAb fragment, a fragment containing a CDR, or an isolated CDR. In certain embodiments, the term “antigen-binding fragment” refers to a polypeptide fragment of a multi-specific antigen-binding molecule. Antigen-binding fragments of an antibody may be derived, e.g., from full antibody molecules using any suitable standard techniques such as proteolytic digestion or recombinant genetic engineering techniques involving the manipulation and expression of DNA encoding antibody variable and (optionally) constant domains. Such DNA is known and/or is readily available from, e.g., commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

Non-limiting examples of antigen-binding fragments include: (i) Fab fragments; (ii) F(ab′)2 fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated complementarity determining region (CDR) such as a CDR3 peptide), or a constrained FR3-CDR3-FR4 peptide. Other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g., monovalent nanobodies, bivalent nanobodies, etc.), small modular immunopharmaceuticals (SMIPs), and shark variable IgNAR domains, are also encompassed within the expression “antigen-binding fragment,” as used herein.

An antigen-binding fragment of an antibody will typically comprise at least one variable domain. The variable domain may be of any size or amino acid composition and will generally comprise at least one CDR, which is adjacent to or in frame with one or more framework sequences. In antigen-binding fragments having a V_{H} domain associated with a V_{L} domain, the V_{H} and V_{L} domains may be situated relative to one another in any suitable arrangement. For example, the variable region may be dimeric and contain V_{H}-V_{H}, V_{H}-V_{L} or V_{L}-V_{L} dimers. Alternatively, the antigen-binding fragment of an antibody may contain a monomeric V_{H} or V_{L} domain.

In certain embodiments, an antigen-binding fragment of an antibody may contain at least one variable domain covalently linked to at least one constant domain. Non-limiting, exemplary configurations of variable and constant domains that may be found within an antigen-binding fragment of an antibody of the present invention include: (i) V_{H}-C_{H}1; (ii) V_{H}-C_{H}2; (iii) V_{H}-C_{H}3; (iv) V_{H}-C_{H}1-C_{H}2; (v) V_{H}-C_{H}1-C_{H}2-C_{H}3; (vi) V_{H}-C_{H}2-C_{H}3; (vii) V_{H}-C_{L}; (viii) V_{L}-C_{H}1; (ix) V_{L}-C_{H}2; (x) V_{L}—C_{H}3; (xi) V_{L}-C_{H}1-C_{H}2; (xii) V_{L}-C_{H}1-C_{H}2—C_{H}3; (xiii) V_{L}-C_{H}2-C_{H}3; and (xiv) V_{L}-C_{L}. In any configuration of variable and constant domains, including any of the exemplary configurations listed above, the variable and constant domains may be either directly linked to one another or may be linked by a full or partial hinge or linker region. A hinge region may consist of at least 2 (e.g., 5, 10, 15, 20, 40, 60 or more) amino acids, which result in a flexible or semi-flexible linkage between adjacent variable and/or constant domains in a single polypeptide molecule. Moreover, an antigen-binding fragment of an antibody of the present invention may comprise a homo-dimer or hetero-dimer (or other multimer) of any of the variable and constant domain configurations listed above in non-covalent association with one another and/or with one or more monomeric V_{H} or V_{L} domain (e.g., by disulfide bond(s)).

As with full antibody molecules, antigen-binding fragments may be mono-specific or multi-specific (e.g., bi-specific). A multi-specific antigen-binding fragment of an antibody will typically comprise at least two different variable domains, wherein each variable domain is capable of specifically binding to a separate antigen or to a different epitope on the same antigen. Any multi-specific antibody format, including the exemplary bi-specific antibody formats disclosed herein, may be adapted for use in the context of an antigen-binding fragment of an antibody of the present invention using routine techniques available in the art.

### Preparation of Human Antibodies

Methods for generating human antibodies in transgenic mice are known in the art. Any such known methods can be used in the context of the present invention to make human bispecific antigen-binding molecules, human bispecific antibodies that specifically bind to human GDF8 and human Activin A.

Human antibodies against Activin A or GDF8 may be isolated from a full length human IgG synthetic naive library using an in vitro yeast selection system and associated methods (Rouha H, et al. MAbs. 2015; 7(1):243-254). An antibody library of approximately 1×10¹⁰ in diversity may be designed and propagated as described previously (Rouha et al. 2015, Xu et al., Protein Eng Des Sel. 2013; 26(10):663-670). Activin A-binding antibodies or GDF8-binding antibodies may be enriched by incubation of biotinylated Activin A-Fc and Myc-His monomeric Activin A or biotinylated GDF8-Fc and Myc-His monomeric GDF8, respectively, at different concentrations with antibody-expressing yeast cells followed by magnetic bead selection (Miltenyi Biotec) or flow cytometry on a FACSAria II cell sorter (BD Biosciences), for example, using fluorescent streptavidin or extravidin secondary reagents in several successive selection rounds.

Antibodies cross-reactive to off-target proteins Activin B, Inhibin A, Inhibin B, GDF3, GDF11, Nodal, BMP2, BMP4, and/or BMP7, and the like, may be actively depleted from selection outputs. After the last round of enrichment, yeast cells may be plated onto agar plates, analyzed by DNA sequencing, and expanded for IgG production. Heavy chains from the naive outputs may be used to prepare light-chain diversification libraries, which may then used for additional selection rounds. In particular, heavy chains may be extracted from naive selection round outputs and transformed into a light-chain library, for example, consisting of 1×10⁶ unique light chains to create new libraries of, for example, approximately 1×10⁸ in total diversity. Antibody optimization may be completed in 3 phases. Optimization of the heavy chain via diversification of the complementarity-determining regions (CDRs) HCDR1 and HCDR2 may be followed either by mutagenic PCR-based diversification of the entire heavy chain variable region or diversification of the light chain LCDR1 and LCDR2 segments. HCDR1 and HCDR2 regions may be diversified with premade libraries of HCDR1 and HCDR2 variants of a diversity of, for example, approximately 1×10⁸. Lead variants may be further diversified via DNA oligonucleotide sequence variegation of the HCDR3 or LCDR3.

Diversified antibody lineage populations may be selected for enhanced binding to the target proteins while avoiding undesired cross-reactivity. The methods used for selections on diversified populations may be similar or identical to those used to isolate the original lead IgGs (Xu et al., 2013).

Alternatively, immunogen comprising any one of the following can be used to generate antibodies to Activin A or GDF8 protein.

In certain embodiments, the antibodies of the invention are obtained from mice immunized with a human Activin A or a fragment thereof. The Activin A may comprise an Activin beta A subunit protein. (See, for example, UniProtKB/Swiss-Prot accession number P08476) (SEQ ID NO: 40) (or with DNA encoding the protein or fragment thereof). Alternatively, the protein or a fragment thereof may be produced using standard biochemical techniques and modified and used as immunogen. In some embodiments, the immunogen may be a recombinant Activin A protein fragment thereof such as Gly311-Ser426 of UniProtKB/Swiss-Prot accession number P08476 (SEQ ID NO: 43) expressed in E. coli or in any other eukaryotic or mammalian cells such as Chinese hamster ovary (CHO) cells. In some cases, the Activin A protein may include an Activin beta B subunit or fragment thereof. In some cases, the Activin beta B subunit or fragment thereof may comprise the amino acid sequence of SEQ ID NO: 41.

In certain embodiments, the antibodies of the invention are obtained from mice immunized with a GDF8 preprotein (See, for example, UniProtKB/Swiss-Prot accession number 008689) (SEQ ID NO: 39) (or with DNA encoding the protein or fragment thereof). Alternatively, the protein or a fragment thereof may be produced using standard biochemical techniques and modified and used as immunogen. In some embodiments, the immunogen may be a recombinant GDF8 protein or fragment thereof such as protein or an appropriate fragment thereof such as Asp268-Ser376 of UniProtKB/Swiss-Prot accession number 008689 (SEQ ID NO: 42) expressed in E. coli or in any other eukaryotic or mammalian cells such as Chinese hamster ovary (CHO) cells.

Using VELOCIMMUNE® technology (see, for example, U.S. Pat. No. 6,596,541, Regeneron Pharmaceuticals, VELOCIMMUNE®) or any other known method for generating monoclonal antibodies, high affinity chimeric antibodies to GDF8 or Activin A are initially isolated having a human variable region and a mouse constant region. The VELOCIMMUNE® technology involves generation of a transgenic mouse having a genome comprising human heavy and light chain variable regions operably linked to endogenous mouse constant region loci such that the mouse produces an antibody comprising a human variable region and a mouse constant region in response to antigenic stimulation. The DNA encoding the variable regions of the heavy and light chains of the antibody are isolated and operably linked to DNA encoding the human heavy and light chain constant regions. The DNA is then expressed in a cell capable of expressing the fully human antibody.

Generally, a VELOCIMMUNE® mouse is challenged with the antigen of interest, and lymphatic cells (such as B-cells) are recovered from the mice that express antibodies. The lymphatic cells may be fused with a myeloma cell line to prepare immortal hybridoma cell lines, and such hybridoma cell lines are screened and selected to identify hybridoma cell lines that produce antibodies specific to the antigen of interest. DNA encoding the variable regions of the heavy chain and light chain may be isolated and linked to desirable isotypic constant regions of the heavy chain and light chain. Such an antibody protein may be produced in a cell, such as a CHO cell. Alternatively, DNA encoding the antigen-specific chimeric antibodies or the variable domains of the light and heavy chains may be isolated directly from antigen-specific lymphocytes.

Initially, high affinity chimeric antibodies are isolated having a human variable region and a mouse constant region. As in the experimental section below, the antibodies are characterized and selected for desirable characteristics, including affinity, selectivity, epitope, etc. The mouse constant regions are replaced with a desired human constant region to generate the fully human antibody of the invention, for example wild type or modified IgG1 or IgG4. While the constant region selected may vary according to specific use, high affinity antigen-binding and target specificity characteristics reside in the variable region.

### Bioequivalents

The bispecific antigen-binding molecules and fragments thereof of the present invention encompass proteins having amino acid sequences that vary from those of the described antibodies, but that retain the ability to bind human GDF8 and human Activin A proteins. Such variant antibodies and antibody fragments comprise one or more additions, deletions, or substitutions of amino acids when compared to parent sequence, but exhibit biological activity that is essentially equivalent to that of the described antibodies. Likewise, the antibody-encoding DNA sequences of the present invention encompass sequences that comprise one or more additions, deletions, or substitutions of nucleotides when compared to the disclosed sequence, but that encode an antibody or antibody fragment that is essentially bioequivalent to an antibody or antibody fragment of the invention.

Two antigen-binding proteins, or antibodies, are considered bioequivalent if, for example, they are pharmaceutical equivalents or pharmaceutical alternatives whose rate and extent of absorption do not show a significant difference when administered at the same molar dose under similar experimental conditions, either single dose or multiple doses. Some antibodies will be considered equivalents or pharmaceutical alternatives if they are equivalent in the extent of their absorption but not in their rate of absorption and yet may be considered bioequivalent because such differences in the rate of absorption are intentional and are reflected in the labeling, are not essential to the attainment of effective body drug concentrations on, e.g., chronic use, and are considered medically insignificant for the particular drug product studied.

In one embodiment, two antigen-binding proteins are bioequivalent if there are no clinically meaningful differences in their safety, purity, or potency.

In one embodiment, two antigen-binding proteins are bioequivalent if a patient can be switched one or more times between the reference product and the biological product without an expected increase in the risk of adverse effects, including a clinically significant change in immunogenicity, or diminished effectiveness, as compared to continued therapy without such switching.

In one embodiment, two antigen-binding proteins are bioequivalent if they both act by a common mechanism or mechanisms of action for the condition or conditions of use, to the extent that such mechanisms are known.

Bioequivalence may be demonstrated by in vivo and/or in vitro methods. Bioequivalence measures include, e.g., (a) an in vivo test in humans or other mammals, in which the concentration of the antibody or its metabolites is measured in blood, plasma, serum, or other biological fluid as a function of time; (b) an in vitro test that has been correlated with and is reasonably predictive of human in vivo bioavailability data; (c) an in vivo test in humans or other mammals in which the appropriate acute pharmacological effect of the antibody (or its target) is measured as a function of time; and (d) in a well-controlled clinical trial that establishes safety, efficacy, or bioavailability or bioequivalence of an antibody.

Bioequivalent variants of the antibodies of the invention may be constructed by, for example, making various substitutions of residues or sequences or deleting terminal or internal residues or sequences not needed for biological activity. For example, cysteine residues not essential for biological activity can be deleted or replaced with other amino acids to prevent formation of unnecessary or incorrect intramolecular disulfide bridges upon renaturation. In other contexts, bioequivalent antibodies may include antibody variants comprising amino acid changes, which modify the glycosylation characteristics of the antibodies, e.g., mutations that eliminate or remove glycosylation.

### Antibodies Comprising Fc Variants

According to certain embodiments of the present invention, bispecific antigen-binding proteins comprising a first antigen-binding domain that specifically binds to GDF8 and a second antigen-binding domain that specifically binds to Activin A such as bispecific antibodies are provided comprising an Fc domain comprising one or more mutations which enhance or diminish antibody binding to the FcRn receptor, e.g., at acidic pH as compared to neutral pH. For example, the present invention includes bispecific antibodies comprising a mutation in the C_{H2} or a CH³ region of the Fc domain, wherein the mutation(s) increases the affinity of the Fc domain to FcRn in an acidic environment (e.g., in an endosome where pH ranges from about 5.5 to about 6.0). Such mutations may result in an increase in serum half-life of the antibody when administered to an animal. Non-limiting examples of such Fc modifications include, e.g., a modification at position 250 (e.g., E or Q); 250 and 428 (e.g., L or F); 252 (e.g., L/Y/F/W or T), 254 (e.g., S or T), and 256 (e.g., S/R/Q/E/D or T); or a modification at position 428 and/or 433 (e.g., H/L/R/S/P/Q or K) and/or 434 (e.g., A, W, H, F or Y [N434A, N434W, N434H, N434F or N434Y]); or a modification at position 250 and/or 428; or a modification at position 307 or 308 (e.g., 308F, V308F), and 434. In one embodiment, the modification comprises a 428L (e.g., M428L) and 434S (e.g., N434S) modification; a 428L, 259I (e.g., V259I), and 308F (e.g., V308F) modification; a 433K (e.g., H433K) and a 434 (e.g., 434Y) modification; a 252, 254, and 256 (e.g., 252Y, 254T, and 256E) modification; a 2500 and 428L modification (e.g., T250Q and M428L); and a 307 and/or 308 modification (e.g., 308F or 308P). In yet another embodiment, the modification comprises a 265A (e.g., D265A) and/or a 297A (e.g., N297A) modification.

For example, the present invention includes GDF8 x Activin A bispecific antibodies comprising an Fc domain comprising one or more pairs or groups of mutations selected from the group consisting of: 2500 and 248L (e.g., T250Q and M248L); 252Y, 254T and 256E (e.g., M252Y, S254T and T256E); 428L and 434S (e.g., M428L and N434S); 2571 and 3111 (e.g., P2571 and Q3111); 2571 and 434H (e.g., P2571 and N434H); 376V and 434H (e.g., D376V and N434H); 307A, 380A and 434A (e.g., T307A, E380A and N434A); and 433K and 434F (e.g., H433K and N434F).

All possible combinations of the foregoing Fc domain mutations and other mutations within the antibody variable domains disclosed herein, are contemplated within the scope of the present invention.

The present invention also includes GDF8 x Activin A bispecific antibodies comprising a chimeric heavy chain constant (C_{H}) region, wherein the chimeric C_{H} region comprises segments derived from the C_{H} regions of more than one immunoglobulin isotype. For example, the antibodies of the invention may comprise a chimeric C_{H} region comprising part or all of a C_{H}2 domain derived from a human IgG1, human IgG2 or human IgG4 molecule, combined with part or all of a C_{H}3 domain derived from a human IgG1, human IgG2 or human IgG4 molecule. According to certain embodiments, the antibodies of the invention comprise a chimeric C_{H} region having a chimeric hinge region. For example, a chimeric hinge may comprise an “upper hinge” amino acid sequence (amino acid residues from positions 216 to 227 according to EU numbering) derived from a human IgG1, a human IgG2 or a human IgG4 hinge region, combined with a “lower hinge” sequence (amino acid residues from positions 228 to 236 according to EU numbering) derived from a human IgG1, a human IgG2 or a human IgG4 hinge region. According to certain embodiments, the chimeric hinge region comprises amino acid residues derived from a human IgG1 or a human IgG4 upper hinge and amino acid residues derived from a human IgG2 lower hinge. An antibody comprising a chimeric C_{H} region as described herein may, in certain embodiments, exhibit modified Fc effector functions without adversely affecting the therapeutic or pharmacokinetic properties of the antibody. (See, e.g., U.S. Patent Application Publication 2014/0243504, the disclosure of which is hereby incorporated by reference in its entirety).

### Biological Characteristics of the Antibodies

The present disclosure provides multi-specific antigen-binding molecules comprising a first domain that specifically binds to human GDF8 and a second domain that specifically binds to Activin A such as a GDF8 x Activin A bispecific antibody.

In general, the multi-specific antigen-binding molecules of the present invention function by binding to Activin A protein and decreasing its activity and bind to GDF8 protein and decrease its activity. For example, the present invention includes bispecific antigen-binding molecules and antigen-binding fragments thereof that bind human Activin A protein and GDF8 protein (e.g., at 25° C. or at 37° C.) with a K_{D} of less than 50 nM as measured by surface plasmon resonance, e.g., using the assay format as defined in Example 3 herein.

In certain embodiments, the GDF8 x Activin A bispecific antigen-binding molecules or antigen-binding fragments thereof bind GDF8 with a K_{D} of less than about 50 nM, less than about 40 nM, less than about 30 nM, less than about 20 nM, less than about 10 nM, less than about 5 nM, less than about 2.5 nM, less than about 1 nM, or less than about 0.5 nM, less than about 0.3 nM, less than about 0.2 nM, less than about 0.1 nM, less than about 50 pM, less than about 20 pM, or less than about 15 pM as measured by surface plasmon resonance at 25° C., e.g., using the assay format as defined in Example 3 herein, or a substantially similar assay.

In certain embodiments, the GDF8 x Activin A bispecific antigen-binding molecules or antigen-binding fragments thereof bind human GDF8 with a K_{D} of less than about 50 nM, less than about 40 nM, less than about 30 nM, less than about 20 nM, less than about 10 nM, less than about 5 nM, less than about 2.5 nM, less than about 1 nM, or less than about 0.5 nM, less than about 0.3 nM, less than about 0.2 nM, less than about 0.1 nM, less than about 50 pM, less than about 20 pM, or less than about 15 pM as measured by surface plasmon resonance at 37° C., e.g., using the assay format as defined in Example 3 herein, or a substantially similar assay.

In certain embodiments, the GDF8 x Activin A bispecific antigen-binding molecules or antigen-binding fragments thereof bind human Activin A with a K_{D} of less than about 50 nM, less than about 40 nM, less than about 30 nM, less than about 20 nM, less than about 10 nM, less than about 5 nM, less than about 2.5 nM, less than about 1 nM, or less than about 0.5 nM, less than about 0.3 nM, less than about 0.2 nM, less than about 0.1 nM, or less than about 50 pM as measured by surface plasmon resonance at 25° C., e.g., using the assay format as defined in Example 3 herein, or a substantially similar assay.

In certain embodiments, the GDF8 X Activin A bispecific antigen-binding molecules or antigen-binding fragments thereof bind human Activin A with a K_{D} of less than about 50 nM, less than about 40 nM, less than about 30 nM, less than about 20 nM, less than about 10 nM, less than about 5 nM, less than about 2.5 nM, less than about 1 nM, or less than about 0.5 nM, less than about 0.3 nM, less than about 0.2 nM, less than about 0.1 nM as measured by surface plasmon resonance at 37° C., e.g., using the assay format as defined in Example 3 herein, or a substantially similar assay.

In certain embodiments, the present invention provides an isolated GDF8 x Activin A bispecific antigen binding molecule or antigen-binding fragment thereof that is a fully human monoclonal antibody.

The present invention also includes bispecific antigen binding molecules and antigen-binding fragments thereof that inhibit Activin A-mediated cellular signaling and GDF8-activated cellular signaling.

For example, the present invention includes GDF8 x Activin A bispecific antigen binding molecules and antigen-binding fragments that inhibit activation of the SMAD complex signal transduction pathway via the binding of Activin A and/or GDF8 to Activin Type II receptors with an IC₅₀ value of less than about 20 nM, as measured in a cell-based blocking bioassay, e.g., using the assay format as defined in Example 4 herein, or a substantially similar assay.

In certain embodiments, the GDF8 x Activin A bispecific antigen binding molecules and antigen-binding fragments of the present invention inhibit activation of the SMAD complex signal transduction pathway via the binding of Activin A to Activin Type II receptors (e.g., ActRIIA) with an IC₅₀ value of less than about 10 nM, less than about 5 nM, less than about 4 nm, less than about 500 pM, less than about 250 pM, less than about 240 pM, less than about 230 pM, less than about 220 pM, less than about 210 pM, less than about 200 pM, less than about 190 pM, less than about 180 pM, as measured in a cell-based blocking bioassay, e.g., using the assay format as defined in Example 4 herein, or a substantially similar assay.

In certain embodiments, the GDF8 x Activin A bispecific antigen binding molecules and antigen-binding fragments of the present invention inhibit activation of the SMAD complex signal transduction pathway via the binding of GDF8 to Activin Type II receptors (e.g., ActRIIB) with an IC₅₀ value of less than about 15 nM, less than about 10 nM, or less than about 8 nm as measured in a cell-based blocking bioassay, e.g., using the assay format as defined in Example 4 herein, or a substantially similar assay.

The invention also includes GDF8 x Activin A bispecific antigen binding molecules and antigen-binding fragments thereof that induce muscle hypertrophy in vivo.

The invention also includes GDF8 x Activin A bispecific antigen binding molecules and antigen-binding fragments thereof that induce significant muscle hypertrophy in vivo in one or more of tibialis anterior (TA) muscle, quadriceps muscle, gastrocnemius muscle (GA) and soleus muscles.

The invention also includes GDF8 x Activin A bispecific antigen binding molecules and antigen-binding fragments thereof that induce significant muscle hypertrophy in vivo but do not induce significant decrease in liver weight.

The invention also includes GDF8 x Activin A bispecific antigen binding molecules and antigen-binding fragments thereof that induce significant muscle hypertrophy in vivo but do not induce significant increase in heart weight.

The invention also includes GDF8 x Activin A bispecific antigen binding molecules and antigen-binding fragments thereof that do not significantly increase subcutaneous white adipose (scWAT), or gonadal white adipose tissue (gWAT) in vivo.

The invention also includes GDF8 x Activin A bispecific antigen binding molecules and antigen-binding fragments thereof that decrease subcutaneous white adipose (scWAT), and gonadal white adipose tissue (gWAT) in vivo.

The invention also includes GDF8 x Activin A bispecific antigen binding molecules and antigen-binding fragments thereof that bind and block the activity of Activin A and GDF8.

In certain embodiments, the GDF8 x Activin A bispecific antigen binding molecules and antigen-binding fragments thereof specifically bind human Activin A or a fragment thereof and GDF8 or a fragment thereof, and comprise a HCVR comprising an amino acid sequence selected from the group consisting of HCVR sequence listed in Table 1 and a LCVR comprising an amino acid sequence selected from the group consisting of LCVR sequences listed in Table 1.

In one embodiment, the present invention provides an isolated recombinant bispecific antibody or antigen-binding fragment thereof that binds specifically to GDF8 protein and inhibits GDF8-mediated signal transduction and binds specifically to Activin A and inhibits Activin A-mediated signal transduction, wherein the bispecific antibody or fragment thereof exhibits one or more of the following characteristics:
(a) is a fully human monoclonal antibody;
(b) binds to GDF8 protein Asp268-Ser376 of UniProtKB/Swiss-Prot accession number 008689 (SEQ ID NO: 42) at 25° C. with a dissociation constant (K_{D}) of less than about 100 pM, less than about 70 pM, less than about 50 pM, less than about 25 pM, less than about 20 pM, or less than about 15 pM, as measured in a surface plasmon resonance assay;
(c) binds to GDF8 protein Asp268-Ser376 of UniProtKB/Swiss-Prot accession number 008689 (SEQ ID NO: 42) at 37° C. with a dissociation constant (K_{D}) of less than about 100 pM, less than about 70 pM, less than about 50 pM, less than about 25 pM, less than about 20 pM, less than about 15 pM, or less than about 12 pM as measured in a surface plasmon resonance assay;
(d) binds to Activin A protein Gly311-Ser426 of UniProtKB/Swiss-Prot accession number P08476 (SEQ ID NO: 43) at 25° C. with a dissociation constant (K_{D}) of less than about 250 pM, less than about 225 pM, less than about 100 pM, less than about 50 pM, less than about 25 pM, or less than about 20 pM as measured in a surface plasmon resonance assay;
(e) binds to Activin A protein Gly311-Ser426 of UniProtKB/Swiss-Prot accession number P08476 (SEQ ID NO: 43) at 37° C. with a dissociation constant (K_{D}) of less than about 1 nM, less than about 750 pM, less than about 500 pM, less than about 200 pM, less than about 100 pM, lessthan about 50 pM, or less than about 25 pM as measured in a surface plasmon resonance assay;
(f) inhibits activation of cells expressing SMAD 2/3 transcription factors by human Activin A with a IC₅₀ of less than 5 nM, less than 3 nM, less than 2 nM, less than 1 nM, less than 0.5 nM, less than 0.25 nM, or less than 0.2 nM as measured in a cell-based bioassay;
(g) inhibits activation of cells expressing SMAD 2/3 transcription factors by GDF8 with a IC₅₀ of less than 20 nM, less than 15 nM, less than 10 nM, less than 8 nM, or less than 7 nM as measured in a cell-based bioassay; and
(h) comprises (i) an HCVR of an antibody that binds GDF8, wherein the HCVR comprises an amino acid sequence selected from the group consisting of sequences listed in Table 1, (ii) an HCVR of an antibody that binds Activin A, wherein the HCVR comprises an amino acid sequence selected from the group consisting of sequences listed in Table 1; and (iii) an LCVR of an antibody that binds GDF8, wherein the LCVR comprises an amino acid sequence selected from the group consisting of sequences listed in Table 1.

The antibodies of the present invention may possess one or more of the aforementioned biological characteristics, or any combinations thereof. Other biological characteristics of the antibodies of the present invention will be evident to a person of ordinary skill in the art from a review of the present disclosure including the working Examples herein.

### Epitope Mapping and Related Technologies

The present invention includes bispecific antigen-binding molecule comprising a GDF8-specific binding domain and an Activin A-specific binding domain which interact with one or more amino acids found within one or more regions of the Activin A protein molecule and GDF8 protein molecule. Each epitope to which the GDF8 x Activin A bispecific antigen-binding molecules bind may consist of a single contiguous sequence of 3 or more (e.g., 3, 4, 5, 6, 7, 8, 9, 10, 11, 12, 13, 14, 15, 16, 17, 18, 19, 20 or more) amino acids located within any of the aforementioned domains of the Activin A protein molecule or GDF8 molecule (e.g. a linear epitope in a domain). Alternatively, the epitopes may independently consist of a plurality of non-contiguous amino acids (or amino acid sequences) located within either or both of the aforementioned domains of the protein molecules (e.g. a conformational epitope).

Various techniques known to persons of ordinary skill in the art can be used to determine whether a bispecific antigen-binding molecule “interacts with one or more amino acids” within a polypeptide or protein. Exemplary techniques include, for example, routine cross-blocking assays, such as that described in Antibodies, Harlow and Lane (Cold Spring Harbor Press, Cold Spring Harbor, NY). Other methods include alanine scanning mutational analysis, peptide blot analysis (Reineke (2004) Methods Mol. Biol. 248: 443-63), peptide cleavage analysis crystallographic studies and NMR analysis. In addition, methods such as epitope excision, epitope extraction and chemical modification of antigens can be employed (Tomer (2000) Prot. Sci. 9: 487-496). Another method that can be used to identify the amino acids within a polypeptide with which an antigen-binding molecule interacts is hydrogen/deuterium exchange detected by mass spectrometry. In general terms, the hydrogen/deuterium exchange method involves deuterium-labeling the protein of interest, followed by binding the antigen-binding molecule to the deuterium-labeled protein. Next, the protein/antigen-binding molecule complex is transferred to water and exchangeable protons within amino acids that are protected by the antibody complex undergo deuterium-to-hydrogen back-exchange at a slower rate than exchangeable protons within amino acids that are not part of the interface. As a result, amino acids that form part of the protein/antigen-binding molecule interface may retain deuterium and therefore exhibit relatively higher mass compared to amino acids not included in the interface. After dissociation of the antigen-binding molecule, the target protein is subjected to protease cleavage and mass spectrometry analysis, thereby revealing the deuterium-labeled residues which correspond to the specific amino acids with which the antigen-binding molecule interacts. See, e.g., Ehring (1999) Analytical Biochemistry 267: 252-259; Engen and Smith (2001) Anal. Chem. 73: 256A-265A.

Modification-Assisted Profiling (MAP), also known as Antigen Structure-based Antibody Profiling (ASAP) is a method that categorizes large numbers of antigen-binding molecules such as monoclonal antibodies (mAbs) directed against the same antigen according to the similarities of the binding profile of each antibody to chemically or enzymatically modified antigen surfaces (see US 2004/0101920, herein specifically incorporated by reference in its entirety). Each category may reflect a unique epitope either distinctly different from or partially overlapping with epitope represented by another category. This technology allows rapid filtering of genetically identical antibodies, such that characterization can be focused on genetically distinct antibodies. When applied to hybridoma screening, MAP may facilitate identification of rare hybridoma clones that produce mAbs having the desired characteristics. MAP may be used to sort the antibodies of the invention into groups of antibodies binding different epitopes.

In certain embodiments, the present invention includes GDF8 x Activin A bispecific antigen-binding molecules and antigen-binding fragments thereof that interact with one or more epitopes found within the extracellular domain of Activin A or GDF8. The epitopes may consist of one or more contiguous sequences of 3 or more (e.g., 3, 4, 5, 6, 7, 8, 9, 10, 11, 12, 13, 14, 15, 16, 17, 18, 19, 20 or more) amino acids located within the extracellular domain of Activin A or GDF8. Alternatively, the epitope may consist of a plurality of non-contiguous amino acids (or amino acid sequences) located within the Activin A or GDF8 protein.

The present invention includes multi-specific antigen-binding molecules comprising a GDF8-specific binding domain and an Activin A-specific binding domain that bind to the same epitope, or a portion of the epitope, as any of the specific exemplary bispecific antigen-binding molecules listed in Table 1. Likewise, the present invention also includes multi-specific antigen-binding molecules comprising a GDF8-specific binding domain and an Activin A-specific binding domain that compete for binding to Activin A or GDF8 protein or a fragment thereof with any of the specific exemplary bispecific antigen-binding molecules comprising a GDF8-specific binding domain and an Activin A-specific binding domain listed in Table 1. For example, the present invention includes multi-specific antigen-binding molecules comprising a GDF8-specific binding domain and an Activin A-specific binding domain that cross-compete for binding to Activin A and/or GDF8 with one or more bispecific antigen-binding molecules listed in Table 1.

One can easily determine whether an antibody binds to the same epitope as, or competes for binding with, a reference bispecific antigen-binding molecule by using routine methods known in the art. For example, to determine if a test bispecific antigen-binding molecules binds to the same epitope as a reference multi-specific antigen-binding molecule comprising a GDF8-specific binding domain and an Activin A-specific binding domain of the invention, the reference multi-specific antigen-binding molecule is allowed to bind to a Activin A or GDF8 protein or peptide under saturating conditions. Next, the ability of a test bispecific antigen-binding molecule to bind to the Activin A or GDF8 protein molecule is assessed. If the test bispecific antigen-binding molecule is able to bind to Activin A or GDF8 following saturation binding with the reference multi-specific antigen-binding molecule, it can be concluded that the test bispecific antigen-binding molecule binds to a different epitope than the reference multi-specific antigen-binding molecule. On the other hand, if the test bispecific antigen-binding molecule is not able to bind to the Activin A or GDF8 protein following saturation binding with the reference multi-specific antigen-binding molecule, then the test bispecific antigen-binding molecule may bind to the same epitope as the epitope bound by the reference multi-specific antigen-binding molecule comprising a GDF8-specific binding domain and an Activin A-specific binding domain of the invention.

To determine if a bispecific antigen-binding molecule competes for binding with a reference multi-specific antigen-binding molecule comprising a GDF8-specific binding domain and an Activin A-specific binding domain, the above-described binding methodology is performed in two orientations: In a first orientation, the reference multi-specific antigen-binding molecule is allowed to bind to a Activin A or GDF8 protein under saturating conditions followed by assessment of binding of the test bispecific antigen-binding molecule to the Activin A or GDF8 molecule. In a second orientation, the test bispecific antigen-binding molecule is allowed to bind to a Activin A or GDF8 molecule under saturating conditions followed by assessment of binding of the reference multi-specific antigen-binding molecule to the Activin A or GDF8 molecule. If, in both orientations, only the first (saturating) antibody is capable of binding to the Activin A or GDF8 molecule, then it is concluded that the test bispecific antigen-binding molecule and the reference multi-specific antigen-binding molecule compete for binding to Activin A or GDF8. As will be appreciated by a person of ordinary skill in the art, an bispecific antigen-binding molecule that competes for binding with a reference multi-specific antigen-binding molecule may not necessarily bind to the identical epitope as the reference, but may sterically block binding of the reference multi-specific antigen-binding molecule by binding an overlapping or adjacent epitope.

Two antibodies bind to the same or overlapping epitope if each competitively inhibits (blocks) binding of the other to the antigen. That is, a 1-, 5-, 10-, 20- or 100-fold excess of one antigen-binding molecule inhibits binding of the other by at least 50% but preferably 75%, 90% or even 99% as measured in a competitive binding assay (see, e.g., Junghans et al., Cancer Res. 1990 50:1495-1502). Alternatively, two multi-specific antigen-binding molecules have the same epitope if essentially all amino acid mutations in the antigen that reduce or eliminate binding of one multi-specific antigen-binding molecule reduce or eliminate binding of the other. Two multi-specific antigen-binding molecules have overlapping epitopes if some amino acid mutations that reduce or eliminate binding of one multi-specific antigen-binding molecule reduce or eliminate binding of the other.

Additional routine experimentation (e.g., peptide mutation and binding analyses) can then be carried out to confirm whether the observed lack of binding of the test antigen-binding molecule is in fact due to binding to the same epitope as the reference antigen-binding molecule or if steric blocking (or another phenomenon) is responsible for the lack of observed binding. Experiments of this sort can be performed using ELISA, RIA, surface plasmon resonance, flow cytometry or any other quantitative or qualitative antibody-binding assay available in the art.

### Species Selectivity and Species Cross-Reactivity

The present invention, according to certain embodiments, provides GDF8 x Activin A bispecific antibodies that bind to human Activin A but not to Activin A from other species. The present invention also includes GDF8 x Activin A bispecific antibodies that bind to human Activin A and to Activin A from one or more non-human species. For example, the anti-Activin A antibodies of the invention may bind to human Activin A and may bind or not bind, as the case may be, to one or more of mouse, rat, guinea pig, hamster, gerbil, pig, cat, dog, rabbit, goat, sheep, cow, horse, camel, cynomologous, marmoset, rhesus or chimpanzee Activin A. According to certain exemplary embodiments of the present invention, anti-Activin A antibodies are provided which specifically bind human Activin A (e.g., Activin A or a βA subunit-containing heterodimer) and cynomolgus monkey (e.g., Macaca fascicularis) Activin A.

### Immunoconjugates

The invention encompasses a human multi-specific antigen-binding molecule comprising a GDF8-specific binding domain and an Activin A-specific binding domain conjugated to a therapeutic moiety (“immunoconjugate”), to treat an Activin A-associated disease or disorder or GDF8-associated disease or disorder. As used herein, the term “immunoconjugate” refers to an multi-specific antigen-binding molecule which is chemically or biologically linked to a radioactive agent, a cytokine, an interferon, a target or reporter moiety, an enzyme, a peptide or protein or a therapeutic agent. The multi-specific antigen-binding molecule may be linked to the radioactive agent, cytokine, interferon, target or reporter moiety, enzyme, peptide or therapeutic agent at any location along the molecule so long as it is able to bind its target. Examples of immunoconjugates include multi-specific antigen-binding molecule drug conjugates and multi-specific antigen-binding molecule-toxin fusion proteins. In one embodiment, the agent may be a second different antibody to Activin A or GDF8 protein. The type of therapeutic moiety that may be conjugated to the multi-specific antigen-binding molecule will take into account the condition to be treated and the desired therapeutic effect to be achieved. Examples of suitable agents for forming immunoconjugates are known in the art; see for example, WO 05/103081, which is herein incorporated by reference.

### Multi-Specific Antigen-Binding Molecules

The multi-specific antigen-binding molecule comprising a GDF8-specific binding domain and an Activin A-specific binding domain of the present invention may be bi-specific or multi-specific. Multi-specific antibodies may be specific for different epitopes of one target polypeptide or may contain antigen-binding domains specific for more than one target polypeptide. See, e.g., Tutt et al., 1991, J. Immunol. 147:60-69; Kufer et al., 2004, Trends Biotechnol. 22:238-244, each of which is herein incorporated by reference.

Any of the GDF8 x Activin A multi-specific antigen-binding molecules of the invention, or variants thereof, may be constructed using standard molecular biological techniques (e.g., recombinant DNA and protein expression technology), as will be known to a person of ordinary skill in the art.

In some embodiments, multi-specific antigen-binding molecules comprising a GDF8-specific binding domain and an Activin A-specific binding domain are generated in a bi-specific format (a “bi-specific”) in which variable regions binding to distinct domains of activin A protein and GDF8 protein are linked together to confer dual-domain specificity within a single binding molecule. Appropriately designed multi-specifics may enhance overall activin A and/or GDF8 inhibitory efficacy through increasing both specificity and binding avidity. Variable regions with specificity for individual domains, (e.g., segments of the N-terminal domain), or that can bind to different regions within one domain, are paired on a structural scaffold that allows each region to bind simultaneously to the separate epitopes, or to different regions within one domain. In one example for a bi-specific, heavy chain variable regions (V_{H}) from a binder with specificity for one domain are recombined with light chain variable regions (V_{L}) from a series of binders with specificity for a second domain to identify non-cognate V_{L} partners that can be paired with an original V_{H} without disrupting the original specificity for that V_{H}. In this way, a single V_{L} segment (e.g., V_{L}1) can be combined with two different V_{H} domains (e.g., V_{H}1 and V_{H}2) to generate a bi-specific comprised of two binding “arms” (V_{H}1-V_{L}1 and V_{H}2-V_{L}1). Use of a single V_{L} segment reduces the complexity of the system and thereby simplifies and increases efficiency in cloning, expression, and purification processes used to generate the bi-specific (See, for example, US2011/0195454 and US2010/0331527).

Alternatively, multi-specific antigen-binding molecules that bind more than one domains of Activin A and a second target, such as, but not limited to, for example, a second different GDF8, may be prepared in a bi-specific format using techniques described herein, or other techniques known to those skilled in the art. Antibody variable regions binding to distinct regions may be linked together with variable regions that bind to relevant sites on, for example, to confer dual-antigen specificity within a single binding molecule. Appropriately designed bi-specifics of this nature serve a dual function. Variable regions with specificity for the extracellular domain are combined with a variable region with specificity for outside the extracellular domain and are paired on a structural scaffold that allows each variable region to bind to the separate antigens.

An exemplary bi-specific antibody format that can be used in the context of the present invention involves the use of a first immunoglobulin (Ig) C_{H3} domain and a second Ig C_{H3} domain, wherein the first and second Ig C_{H3} domains differ from one another by at least one amino acid, and wherein at least one amino acid difference reduces binding of the bi-specific antibody to Protein A as compared to a bi-specific antibody lacking the amino acid difference. In one embodiment, the first Ig C_{H3} domain binds Protein A and the second Ig C_{H3} domain contains a mutation that reduces or abolishes Protein A binding such as an H95R modification (by IMGT exon numbering; H435R by EU numbering). The second C_{H3} may further comprise a Y96F modification (by IMGT; Y436F by EU). Further modifications that may be found within the second C_{H3} include: D16E, L18M, N44S, K52N, V57M, and V821 (by IMGT; D356E, L358M, N384S, K392N, V397M, and V4221 by EU) in the case of IgG1 antibodies; N44S, K52N, and V821 (IMGT; N384S, K392N, and V4221 by EU) in the case of IgG2 antibodies; and Q15R, N44S, K52N, V57M, R69K, E79Q, and V821 (by IMGT; Q355R, N384S, K392N, V397M, R409K, E419Q, and V4221 by EU) in the case of IgG4 antibodies. Variations on the bi-specific antibody format described above are contemplated within the scope of the present invention.

Other exemplary bispecific formats that can be used in the context of the present invention include, without limitation, e.g., scFv-based or diabody bispecific formats, IgG-scFv fusions, dual variable domain (DVD)-Ig, Quadroma, knobs-into-holes, common light chain (e.g., common light chain with knobs-into-holes, etc.), CrossMab, CrossFab, (SEED)body, leucine zipper, Duobody, IgG1/lgG2, dual acting Fab (DAF)-IgG, and Mab² bispecific formats (see, e.g., Klein et al. 2012, mAbs 4:6, 1-11, and references cited therein, for a review of the foregoing formats). Bispecific antibodies can also be constructed using peptide/nucleic acid conjugation, e.g., wherein unnatural amino acids with orthogonal chemical reactivity are used to generate site-specific antibody-oligonucleotide conjugates which then self-assemble into multimeric complexes with defined composition, valency and geometry. (See, e.g., Kazane et al., J. Am. Chem. Soc. [Epub: Dec. 4, 2012]).

Methods for providing a bispecific antibody with a common light chain are known in the art. The bispecific antibody comprising a common light chain may be produced by any appropriate method known in the art. For example, a bispecific antibody comprising a common light chain may be produced by the methods of Brinkmann & Kontermann, The making of bispecific antibodies, MABS, 2017, vol. 9, No. 2, 182-212 or Shiraiwa et al., Engineering a bispecific antibody with a common light chain: identification and optimization of an anti-CD3 epsilon and anti-GPC3 bispecific antibody, ERY974, Methods, vol. 154, 1 Feb. 2019, pp. 10-20.

Methods for producing a bispecific antibody with a common heavy chain are known in the art. For example, a bispecific antibody comprising a common heavy chain may be produced by the methods of Magistrelli et al., Optimizing assembly and production of native bispecific antibodies by codon de-optimization, MABS, 2017, Vol. 9, No. 2, 231-239.

A bispecific antibody can bind two different antigens. Immunoglobulin (IgG) type antibodies have two binding sites with different variable regions. An IgG variable region is made up of a variable light chain sequence (VL) and a variable heavy chain sequence (VH). In some cases, the light chains (LCs) of common light chain antibodies are identical for both variable regions, leaving the heavy chain (HC) for generating different specificities. In some cases, recombinant host cells for production of common LC bispecific antibodies carry genes for both HCs, with the different specificities (A and B) along with one LC gene. A, B, and the light chain may be expressed independently in the host cells, which then assemble into three IgG types-AA, AB, and BB— for secretion into the culture environment. By random assembly, the three types can be produced in a ratio of 1:2:1 (AA, AB, and BB; AB and BA are equivalent) Thus purity of AB with respect to AA and BB would be 50%. See Muller-Spath et al., Purifying common-light chain bispecific antibodies, BioProcess International, May 1, 2013.

### Therapeutic Administration and Formulations

The invention provides therapeutic compositions comprising a polyspecific antigen-binding molecule comprising a first antigen-binding domain (D1) that binds GDF8, and a second antigen-binding domain (D2) that binds inhibin @A and dimers containing inhibin RA, e.g., Activin A, Activin AB, etc. The polyspecific antigen-binding molecule may be a bispecific antigen-binding molecule comprising a GDF8-specific binding domain and an Activin A-specific binding domain.

Therapeutic compositions in accordance with the invention will be administered with suitable pharmaceutically acceptable carriers, excipients, and other agents that are incorporated into formulations to provide improved transfer, delivery, tolerance, and the like. A multitude of appropriate formulations can be found in the formulary known to all pharmaceutical chemists: Remington's Pharmaceutical Sciences, Mack Publishing Company, Easton, PA. These formulations include, for example, powders, pastes, ointments, jellies, waxes, oils, lipids, lipid (cationic or anionic) containing vesicles (such as LIPOFECTIN™), DNA conjugates, anhydrous absorption pastes, oil-in-water and water-in-oil emulsions, emulsions carbowax (polyethylene glycols of various molecular weights), semi-solid gels, and semi-solid mixtures containing carbowax. See also Powell et al. “Compendium of excipients for parenteral formulations” PDA (1998) J Pharm Sci Technol 52:238-311.

The dose of antibody may vary depending upon the age and the size of a subject to be administered, target disease, conditions, route of administration, and the like. When an antigen-binding molecule of the present invention is used for treating a disease or disorder in an adult patient, or for preventing such a disease, it is advantageous to administer the antigen-binding molecule of the present invention normally at a single dose of about 0.1 to about 100 mg/kg body weight. Depending on the severity of the condition, the frequency and the duration of the treatment can be adjusted. In certain embodiments, the antigen-binding molecule of the disclosure can be administered as an initial dose of at least about 0.1 mg to about 800 mg, about 1 to about 600 mg, about 5 to about 500 mg, or about 10 to about 400 mg. In certain embodiments, the initial dose may be followed by administration of a second or a plurality of subsequent doses of the antigen-binding molecule thereof in an amount that can be approximately the same or less than that of the initial dose, wherein the subsequent doses are separated by at least 1 day to 3 days; at least one week, at least 2 weeks; at least 3 weeks; at least 4 weeks; at least 5 weeks; at least 6 weeks; at least 7 weeks; at least 8 weeks; at least 9 weeks; at least 10 weeks; at least 12 weeks; or at least 14 weeks.

Various delivery systems are known and can be used to administer the pharmaceutical composition of the invention, e.g., encapsulation in liposomes, microparticles, microcapsules, recombinant cells capable of expressing the mutant viruses, receptor mediated endocytosis (see, e.g., Wu et al. (1987) J. Biol. Chem. 262:4429-4432). Methods of introduction include, but are not limited to, intradermal, transdermal, intramuscular, intraperitoneal, intravenous, subcutaneous, intranasal, epidural, intracerebroventricular, and oral routes. The composition may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. The pharmaceutical composition can be also delivered in a vesicle, in particular a liposome (see, for example, Langer (1990) Science 249:1527-1533).

The use of nanoparticles to deliver the bispecific antigen-binding molecule comprising a GDF8-specific binding domain and an Activin A-specific binding domain of the present invention is also contemplated herein. Antigen-binding molecule-conjugated nanoparticles may be used both for therapeutic and diagnostic applications. Antibody-conjugated nanoparticles and methods of preparation and use are described in detail by Arruebo, M., et aL. 2009 (“Antibody-conjugated nanoparticles for biomedical applications” in J. Nanomat. Volume 2009, Article ID 439389, 24 pages, doi: 10.1155/2009/439389), incorporated herein by reference. Nanoparticles may be developed and conjugated to antibodies contained in pharmaceutical compositions to target cells. Nanoparticles for drug delivery have also been described in, for example, U.S. Pat. No. 8,257,740, or U.S. Pat. No. 8,246,995, each incorporated herein in its entirety.

In certain situations, the pharmaceutical composition can be delivered in a controlled release system. In one embodiment, a pump may be used. In another embodiment, polymeric materials can be used. In yet another embodiment, a controlled release system can be placed in proximity of the composition's target, thus requiring only a fraction of the systemic dose.

The injectable preparations may include dosage forms for intravenous, subcutaneous, intracranial, intraperitoneal and intramuscular injections, drip infusions, etc. These injectable preparations may be prepared by methods publicly known.

A pharmaceutical composition of the present invention can be delivered subcutaneously or intravenously with a standard needle and syringe. In addition, with respect to subcutaneous delivery, a pen delivery device readily has applications in delivering a pharmaceutical composition of the present invention. Such a pen delivery device can be reusable or disposable. A reusable pen delivery device generally utilizes a replaceable cartridge that contains a pharmaceutical composition. Once all of the pharmaceutical composition within the cartridge has been administered and the cartridge is empty, the empty cartridge can readily be discarded and replaced with a new cartridge that contains the pharmaceutical composition. The pen delivery device can then be reused. In a disposable pen delivery device, there is no replaceable cartridge. Rather, the disposable pen delivery device comes prefilled with the pharmaceutical composition held in a reservoir within the device. Once the reservoir is emptied of the pharmaceutical composition, the entire device is discarded.

In treatment of certain diseases or conditions, it may be necessary to overcome the blood-brain barrier. In certain embodiments, the blood-brain barrier is overcome by using one or more approaches disclosed in the art, e.g., in Parodi et al., 2019, Pharmaceutics 11:245, which is herein incorporated by reference.

Advantageously, the pharmaceutical compositions for oral or parenteral use described above are prepared into dosage forms in a unit dose suited to fit a dose of the active ingredients. Such dosage forms in a unit dose include, for example, tablets, pills, capsules, injections (ampoules), suppositories, etc. The amount of the contained is generally about 5 to about 500 mg per dosage form in a unit dose; especially in the form of injection, it is preferred that the bispecific antigen-binding molecule comprising a GDF8-specific binding domain and an Activin A-specific binding domain is contained in about 5 to about 300 mg and in about 10 to about 300 mg for the other dosage forms.

### Therapeutic Uses of the Antibodies

The bispecific antigen binding molecules of the present invention are useful for the treatment, and/or prevention of a disease or disorder or condition associated with activin A and/or GDF8 for ameliorating at least one symptom associated with such disease, disorder or condition. In certain embodiments, an antibody or antigen-binding fragment thereof of the invention may be administered at a therapeutic dose to a patient with a disease or disorder or condition associated with activin A or GDF8

The present invention provides methods for increasing muscle mass or strength in a subject by administering to the subject a bispecific antigen-binding molecule comprising a GDF8-specific binding domain and an Activin A-specific binding domain.

The present invention also provides methods for increasing muscle mass or strength in a subject by administering to the subject a bispecific antigen-binding molecule comprising a GDF8-specific binding domain and an Activin A-specific binding domain. The methods according to this aspect of the invention are useful for treating diseases or disorders associated with decreased muscle mass, strength, or power, including, e.g., cachexia, sarcopenia, and other muscle-wasting conditions, injury.

Exemplary diseases, disorders and conditions that can be treated with the compositions of the present invention include, but are not limited to, sarcopenia, cachexia (either idiopathic or secondary to other conditions, e.g., cancer, chronic renal failure, or chronic obstructive pulmonary disease), muscle injury, ligament injury, muscle trauma, muscle wasting and muscle atrophy, e.g., muscle atrophy or wasting caused by or associated with disuse, e.g., muscular, immobilization, bed rest, injury, medical treatment or surgical intervention (e.g., hip fracture, hip replacement, knee replacement, and other joint, tendon, or ligament injuries such as tears in the anterior cruciate ligament (ACL) and/or the medial collateral ligament (MCL), etc.), muscular dystrophy (e.g., Myotonic, Duchenne, Becker, Limb-girdle, Facioscapulohumeral (FSHD, also known as Landouzy-Dejerine disease), Congenital, Oculopharyngeal, Distal, Emery-Dreifuss, etc.), glucocorticoid-induced myopathy, stroke rehabilitation (e.g., rehabilitation for stroke hemiparesis) or by necessity of mechanical ventilation. The compositions of the invention may also be used to treat, prevent or ameliorate diseases such as cancer, obesity, diabetes, arthritis, multiple sclerosis, muscular dystrophy, amyotrophic lateral sclerosis, Parkinson's disease, osteoporosis, osteoarthritis, osteopenia, and metabolic syndromes (including, but not limited to diabetes, obesity, nutritional disorders, organ atrophy, chronic obstructive pulmonary disease, and anorexia). Additional diseases, disorders, and conditions that can be prevented, treated and/or ameliorated using compositions of the present invention include sepsis, chronic heart failure, benign and malignant pheochromocytoma, uterine fibroids/leiomyomata, preeclampsia, keloids and hypertrophic scars, and pulmonary artery hypertension.

It is also contemplated herein to use one or more bispecific antigen-binding molecule comprising a GDF8-specific binding domain and an Activin A-specific binding domain of the present disclosure prophylactically to subjects at risk for suffering from a activin A-associated disease or disorder and/or GDF8-associated disease or disorder.

In one embodiment of the invention, the present bispecific antigen-binding molecules comprising a GDF8-specific binding domain and an Activin A-specific binding domain are used for the preparation of a pharmaceutical composition or medicament for treating patients suffering from a disease, disorder or condition disclosed herein. In another embodiment of the invention, the present bispecific antigen-binding molecules comprising a GDF8-specific binding domain and an Activin A-specific binding domain are used as adjunct therapy with any other agent or any other therapy known to those skilled in the art useful for treating or ameliorating a disease, disorder or condition disclosed herein.

### Combination Therapies

Combination therapies may include an antibody of the invention and any additional therapeutic agent that may be advantageously combined with an antibody of the invention, or with a biologically active fragment of an antibody of the invention. The antibodies of the present invention may be combined synergistically with one or more drugs or therapy used to treat an activin A-associated and/or GDF8-associated disease or disorder. In some embodiments, the antibodies of the invention may be combined with a second therapeutic agent to ameliorate one or more symptoms of said disease or condition.

Depending upon the disease, disorder or condition, the antibodies of the present invention may be used in combination with one or more additional therapeutic agents.

The compositions of the present invention may be administered to a subject along with one or more additional therapeutic agents, including, e.g., growth factor inhibitors, immunosuppressants, anti-inflammatory agents, a glucagon-like peptide-1 receptor agonist (GLP-1 agonist), metabolic inhibitors, enzyme inhibitors, and cytotoxic/cytostatic agents. The additional therapeutic agent(s) may be administered prior to, concurrent with, or after the administration of the GDF8 x Activin A bispecific antigen-binding proteins of the present invention.

Exemplary agents that may be advantageously combined with the bispecific antigen binding molecule of the disclosure include, without limitation, other agents that inhibit GDF8 activity, Activin A activity (including other antibodies or antigen-binding fragments thereof, peptide inhibitors, small molecule antagonists, etc.) and/or agents which do not directly bind GDF8 or Activin A but nonetheless interfere with, block or attenuate GDF8-mediated signaling or Activin A-mediated signaling.

Exemplary anti-activin A agents for use with the bispecific antigen binding molecules of the disclosure include a human anti-activin A antibody or antigen-binding fragment thereof (e.g., an anti-activin A antibody comprising any of the HCVR/LCVR or CDR amino acid sequences as set forth in US 2015-0037339 A1 (e.g., H4H10446P2, H4H10442P2, H4H10430P, or H4H10436P2). Sequences of representative anti-Activin A antibodies are shown in Table 13.

TABLE 13
Amino Acid Sequences of anti-Activin A Antibodies

| Antibody | SEQ ID NO: | | | | | | | |
|---|---|---|---|---|---|---|---|---|
| H4H | HCVR | HCDR1 | HCDR2 | HCDR3 | LCVR | LCDR1 | LCDR2 | LCDR3 |
| 10446P2 | 32 | 12 | 34 | 36 | 44 | 45 | 46 | 47 |
| 10442P2 | 10 | 12 | 14 | 16 | 44 | 45 | 46 | 47 |
| 10430P | 48 | 50 | 51 | 52 | 49 | 53 | 54 | 55 |
| 10436P2 | 56 | 58 | 59 | 60 | 57 | 61 | 62 | 63 |

In one embodiment, the secondary therapeutic agent inhibits, interferes, blocks and/or attenuates the activity of another ligand of the ActRIIA and/or ActRIIB receptor (e.g., GDF8, Activin B, Activin AB, Inhibin A, Inhibin B, GDF3, GDF11, Nodal, BMP2, BMP4, and/or BMP7). In one embodiment, the secondary therapeutic agent is another anti-GDF8 antagonist (e.g., a human anti-GDF8 antibody or antigen-binding fragment thereof). Exemplary anti-GDF8 agents for use with the bispecific antigen binding molecules of the disclosure include a human anti-GDF8 antibody or antigen-binding fragment thereof (e.g., an anti-GDF8 antibody comprising any of the HCVR/LCVR or CDR amino acid sequences as set forth in US 2011-0293630 A1 (e.g., H4H1657N2, H4H1669P, or 8D12). For example, H4H1657N2, is an anti-GDF8 antibody with heavy chain complementarity determining regions (HCDRs) of a HCVR comprising SEQ ID NO:2 (e.g., the HCDR1, HCDR2, and HCDR3 sequences set forth in SEQ ID NO: 4, 6, and 8, respectively), and the light chain complementarity determining regions (LCDRs) of a LCVR comprising SEQ ID NO:18 (e.g., the LCDR1, LCDR2, and LCDR3 sequences set forth in SEQ ID NO: 20, 22, and 24)).

Additional therapeutically active components may include growth factor inhibitors, immunosuppressants, anti-inflammatory agents, metabolic inhibitors, GLP-1 agonists, enzyme inhibitors, and cytotoxic/cytostatic agents. Non-limiting examples of GLP-1 agonists include semaglutide, exenatide, dulaglutide, liraglutide, and lixisenatide. Non-limiting examples of the anti-inflammatory drug can include aspirin, diclofenac, indomethacin, ibuprofen, ketoprofen, naproxen, piroxicam, rofecoxib, celecoxib, azathioprine, penicillamine, methotrexate, sulfasalazine, leflunomide, infliximab, and etanercept. The additional therapeutically active component(s) may be administered prior to, concurrent with, or after the administration of the bispecific antigen binding molecule of the present disclosure.

As used herein, the term “in combination with” means that additional therapeutically active component(s) may be administered prior to, concurrent with, or after the administration of the GDF8 X Activin A bispecific antibody of the present invention. The term “in combination with” also includes sequential or concomitant administration of a bispecific antigen binding molecule and a second therapeutic agent.

The additional therapeutically active component(s) may be administered to a subject prior to administration of a GDF8 x Activin A bispecific antigen binding molecule of the present invention. For example, a first component may be deemed to be administered “prior to” a second component if the first component is administered 1 week before, 72 hours before, 60 hours before, 48 hours before, 36 hours before, 24 hours before, 12 hours before, 6 hours before, 5 hours before, 4 hours before, 3 hours before, 2 hours before, 1 hour before, 30 minutes before, or less than 30 minutes before administration of the second component. In other embodiments, the additional therapeutically active component(s) may be administered to a subject after administration of a bispecific antigen binding molecule of the present invention. For example, a first component may be deemed to be administered “after” a second component if the first component is administered 30 minutes after, 1 hour after, 2 hours after, 3 hours after, 4 hours after, 5 hours after, 6 hours after, 12 hours after, 24 hours after, 36 hours after, 48 hours after, 60 hours after, 72 hours after or more after administration of the second component. In yet other embodiments, the additional therapeutically active component(s) may be administered to a subject concurrent with administration of a bispecific antigen binding molecule of the present invention. “Concurrent” administration, for purposes of the present invention, includes, e.g., administration of a bispecific antigen binding molecule and an additional therapeutically active component to a subject in a single dosage form, or in separate dosage forms administered to the subject within about 30 minutes or less of each other. If administered in separate dosage forms, each dosage form may be administered via the same route (e.g., both the bispecific antigen binding molecule and the additional therapeutically active component may be administered intravenously, etc.); alternatively, each dosage form may be administered via a different route (e.g., the bispecific antigen binding molecule may be administered intravenously, and the additional therapeutically active component may be administered orally). In any event, administering the components in a single dosage from, in separate dosage forms by the same route, or in separate dosage forms by different routes are all considered “concurrent administration,” for purposes of the present disclosure. For purposes of the present disclosure, administration of a bispecific antigen binding molecule “prior to”, “concurrent with,” or “after” (as those terms are defined herein above) administration of an additional therapeutically active component is considered administration of a bispecific antigen binding molecule “in combination with” an additional therapeutically active component.

The present invention includes pharmaceutical compositions in which a GDF8 x Activin A bispecific antigen binding molecule of the present invention is co-formulated with one or more of the additional therapeutically active component(s) as described elsewhere herein.

### Diagnostic Uses of the Antibodies

The polyspecific antigen-binding molecules comprising an activin A specific binding domain and a GDF8 specific binding domain of the present disclosure may be used to detect and/or measure activin A and/or GDF8 protein in a sample, e.g., for diagnostic purposes. Some embodiments contemplate the use of one or more polyspecific antigen-binding molecules of the present invention in assays to detect a activin A-associated- and/or GDF8-associated-disease or disorder. Exemplary diagnostic assays for activin A and/or GDF8 may comprise, e.g., contacting a sample, obtained from a patient, with a polyspecific antigen-binding molecules of the invention, wherein the anti-activin A binding domain or anti-GDF8 binding domain is labeled with a detectable label or reporter molecule or used as a capture ligand to selectively isolate activin A and/or GDF8 from patient samples. Alternatively, an unlabeled polyspecific antigen-binding molecule can be used in diagnostic applications in combination with a secondary antibody which is itself detectably labeled. The detectable label or reporter molecule can be a radioisotope, such as ³H, ¹⁴C ³²P, ³⁵S, or ¹²⁵I; a fluorescent or chemiluminescent moiety such as fluorescein isothiocyanate, or rhodamine; or an enzyme such as alkaline phosphatase, β-galactosidase, horseradish peroxidase, or luciferase. Specific exemplary assays that can be used to detect or measure activin A and/or GDF8 in a sample include enzyme-linked immunosorbent assay (ELISA), radioimmunoassay (RIA), and fluorescence-activated cell sorting (FACS).

Samples that can be used in activin A and/or GDF8 diagnostic assays according to the present invention include any tissue or fluid sample obtainable from a patient, which contains detectable quantities of either activin A protein or fragments thereof and/or GDF8 protein or fragments thereof, under normal or pathological conditions. Generally, levels of activin A protein and/or GDF8 protein in a particular sample obtained from a healthy patient (e.g., a patient not afflicted with a disease associated with activin A or GDF8) will be measured to initially establish a baseline, or standard, level of activin A and/or GDF8. This baseline level of activin A and/or GDF8 can then be compared against the levels of activin A and/or GDF8 measured in samples obtained from individuals suspected of having a activin A-associated condition or symptoms associated with such condition or GDF8-associated condition or symptoms associated with such condition.

The polyspecific antigen-binding molecules specific for Activin A protein and GDF8 protein may contain no additional labels or moieties, or they may contain an N-terminal or C-terminal label or moiety. In one embodiment, the label or moiety is biotin. In a binding assay, the location of a label (if any) may determine the orientation of the peptide relative to the surface upon which the peptide is bound. For example, if a surface is coated with avidin, a peptide containing an N-terminal biotin will be oriented such that the C-terminal portion of the peptide will be distal to the surface.

### EXAMPLES

The following examples are put forth so as to provide those of ordinary skill in the art with a complete disclosure and description of how to make and use the methods and compositions of the invention, and are not intended to limit the scope of what the inventors regard as their invention. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, room temperature is about 25° C., and pressure is at or near atmospheric.

### Example 1: Generation of Human Bispecific Antigen-Binding Molecules

Human antibodies against Activin A were generated as described in US 2015-0037339 A1 (e.g., H4H10442P2, H4H10446P2, H4H10430P). Briefly, an immunogen comprising the Activin A protein (inhibin-βA dimer) was administered directly, with an adjuvant to stimulate the immune response, to a VELOCIMMUNE® mouse comprising DNA encoding human Immunoglobulin heavy and kappa light chain variable regions. The antibody immune response was monitored by a Activin A-specific immunoassay. When a desired immune response was achieved splenocytes were harvested and fused with mouse myeloma cells to preserve their viability and form hybridoma cell lines. The hybridoma cell lines were screened and selected to identify cell lines that produce Activin A-specific antibodies. Using this technique several anti-Activin A chimeric antibodies (i.e., antibodies possessing human variable domains and mouse constant domains) were obtained. An exemplary antibody obtained in this manner is H2aM10965N. The human variable domains from the chimeric antibodies were subsequently cloned onto human constant domains to make fully human anti-Activin A antibodies as described herein.

Anti-Activin A antibodies were also isolated directly from antigen-positive B cells without fusion to myeloma cells, as described in US 2007/0280945A1. Using this method, several fully human anti-Activin A antibodies (i.e., antibodies possessing human variable domains and human constant domains) were obtained; exemplary antibodies generated in this manner were designated as follows: H4H10423P, H4H10429P, H4H10430P (REGN2476), H4H10432P2, H4H10440P2, H4H10442P2 (REGN16429), H4H10436P2, and H4H10446P2 (REGN2477).

Human antibodies against human GDF8 were generated as set forth in US 2011-0293630 A1, e.g., H4H1657N2 (REGN1033), H4H1669P, 8D12, 21-E5, and 1A2.

Bispecific antigen-binding molecule H4H11419D comprising a first antigen-binding domain that specifically binds GDF8 and a second antigen-binding domain that specifically binds Activin A was generated by force pairing the heavy chains from H4H1657N2 and H4H10446P2 with the common light chain of H4H1657N2. Bispecific antigen-binding molecule H4H11418D comprising a first antigen-binding domain that specifically binds GDF8 and a second antigen-binding domain that specifically binds Activin A was generated by force pairing the heavy chains from H4H1657N2 and H4H10442P2 with the common light chain of H4H1657N2. Exemplary bispecific antigen-binding molecules were generated and designated as H4H11418D and H4H11419D (REGN18900).

The biological properties of the exemplary bispecific antigen-binding molecules generated in accordance with the methods of this Example are described in detail in the Examples set forth below.

### Example 2: Heavy and Light Chain Variable Region Amino Acid and Nucleotide Sequences

Table 1 sets forth the amino acid sequence identifiers of the heavy and light chain variable regions and CDRs of selected bispecific antigen-binding molecules of the invention. The corresponding nucleic acid sequence identifiers are set forth in Table 2.

TABLE 1
Amino Acid Sequence Identifiers

| Antigen | SEQ ID NO: | | | | | | | | | | | |
|---|---|---|---|---|---|---|---|---|---|---|---|---|
| binding | VH1 | | | | VH2 | | | | VK | | | |
| molecule | HCVR | HCDR1 | HCDR2 | HCDR3 | HCVR | HCDR1 | HCDR2 | HCDR3 | LCVR | LCDR1 | LCDR2 | LCDR3 |
| H4H11418D | 2 | 4 | 6 | 8 | 10 | 12 | 14 | 16 | 18 | 20 | 22 | 24 |
| H4H11419D | 2 | 4 | 6 | 8 | 32 | 12 | 34 | 36 | 18 | 20 | 22 | 24 |

TABLE 2
Nucleic Acid Sequence Identifiers

| Antigen | SEQ ID NO: | | | | | | | | | | | |
|---|---|---|---|---|---|---|---|---|---|---|---|---|
| binding | VH1 | | | | VH2 | | | | VK | | | |
| molecule | HCVR | HCDR1 | HCDR2 | HCDR3 | HCVR | HCDR1 | HCDR2 | HCDR3 | LCVR | LCDR1 | LCDR2 | LCDR3 |
| H4H11418D | 1 | 3 | 5 | 7 | 9 | 11 | 13 | 15 | 17 | 19 | 21 | 23 |
| H4H11419D | 1 | 3 | 5 | 7 | 31 | 11 | 33 | 35 | 17 | 19 | 21 | 23 |

Bispecific antigen-binding molecules referred to herein typically have fully human variable regions, but may have human or mouse constant regions. As will be appreciated by a person of ordinary skill in the art, an antigen-binding molecule such as an antibody having a particular Fc isotype can be converted to an antibody with a different Fc isotype (e.g., an antibody with a mouse IgG1 Fc can be converted to an antibody with a human IgG4, etc.), but in any event, the variable domains (including the CDRs)—which are indicated by the numerical identifiers shown in Tables 1 or 2—will remain the same, and the binding properties to antigen are expected to be identical or substantially similar regardless of the nature of the Fc domain. In certain embodiments, selected antibodies with a mouse IgG1 Fc are converted to antibodies with human IgG4 Fc. In one embodiment, the IgG4 Fc domain comprises 2 or more amino acid changes as disclosed in US20100331527. In one embodiment, the human IgG4 Fc comprises a serine to proline mutation in the hinge region (S108P) to promote dimer stabilization. Unless indicated otherwise, all antibodies used in the following examples comprise a human IgG4 isotype.

Table 3 sets forth the nucleic acid (DNA) and amino acid (PEP) sequence identifiers of the heavy and light chains (HC and LC) of selected bispecific antigen-binding molecules of the invention.

TABLE 3
Sequence Identifiers for Heavy and Light Chains

| | SEQ ID NO: | | | | | |
|---|---|---|---|---|---|---|
| | HC1 | | HC2 | | LC | |
| Antibody | HC | HC | HC | HC | LC | LC |
| Designation | DNA | PEP | DNA | PEP | DNA | PEP |
| H4H11418D | 25 | 26 | 27 | 28 | 29 | 30 |
| H4H11419D | 25 | 26 | 37 | 38 | 29 | 30 |

Antibodies are typically referred to herein according to the following nomenclature: Fc prefix (e.g., “H1 M,” “H2aM,” “H4H”), followed by a numerical identifier (e.g., “11418,” or “11419” as shown in Tables 1-3), followed by a “P,” “P2”, “D”, or “N” suffix. Thus, according to this nomenclature, an antibody may be referred to herein as, e.g., “H4H11418D”, “H4H10446P2,” “H4H10430P,” “H4H1657N2,” etc. The H1 M, H2M and H4H prefixes on the antibody designations used herein indicate the particular Fc region isotype of the antibody. For example, an “H2aM” antibody has a mouse IgG2a Fc, whereas an “H4H” antibody has a human IgG4 Fc. As will be appreciated by a person of ordinary skill in the art, an antibody having a particular Fc isotype can be converted to an antibody with a different Fc isotype (e.g., an antibody with a mouse IgG2a Fc can be converted to an antibody with a human IgG4, etc.), but in any event, the variable domains (including the CDRs)—which are indicated by the numerical identifiers shown in Table 1-2—will remain the same, and the binding properties are expected to be identical or substantially similar regardless of the nature of the Fc domain.

### Example 3: Binding Kinetics of GDF8 x Activin a Bispecific Antibodies to GDF8 and Activin a Reagents Measured at 25° C. And 37° C. As Determined by Surface Plasmon Resonance

Biacore binding kinetics of anti-GDF8 and Activin bivalent parental and GDF8 x Activin A bispecific antibodies to GDF8 and Activin A reagents was measured at 25° C. and 37° C. Parental antibodies are shown in Table 4 and bispecific antibodies are shown in Table 5.

TABLE 4
Anti-Activin A and Anti-GDF8 mAbs

| Antibody | | |
|---|---|---|
| Designation | Description | Lot # |
| H4H10430P | anti-Activin A bivalent | H4H10430P-L4 |
| H4H10446P2 | anti-Activin A bivalent | H4H10446P2-L2 |
| | parental | |
| H4H10442P2 | Anti-Activin A bivalent | 27-R121120 |
| | parental | |
| H4H1657N2 | anti-GDF8 bivalent | 01-100416 |
| | parental | |

TABLE 5
GDF8 × Activin A bispecific antibodies

| GDF8 × Activin A | | | |
|---|---|---|---|
| Bispecifics | VH | VH* | Lot# |
| H4H11418D | H4H1657N2 | H4H10442P2 | H4H11418D-L1 |
| H4H11419D | H4H1657N2 | H4H10446P2 | REGN18900-L1 |

GDF8 reagent was obtained commercially from R&D Systems Catalog number 788-G8/CF from mouse myeloma cell line, NSO-derived GDF-8/myostatin protein comprising Asp268-Ser 376, Accession number 008689 as a disulfide-linked homodimer. Activin A reagent was obtained commercially from R&D Systems Catalog number 338-AC/CF from Chinese Hamster Ovary cell line, CHO-derived Activin A protein Gly311-Ser426 Accession #P08476 as a disulfide-linked homodimer.

Equilibrium dissociation constants (K_{D}) for anti-GDF8 and Activin A bivalent and bispecific mAbs were determined using a real-time surface plasmon resonance (SPR) based Biacore S200 biosensor. All binding studies were performed in composed of 10 mM HEPES, 150 mM NaCl, 3 mM EDTA, 0.05% and 0.05% v/v surfactant Tween-20, pH 7.4 (HBS-ET) running buffer at 25° C. and 37° C. The Biacore CM4 sensor surface was first derivatized by amine coupling with a monoclonal mouse anti-human Fc antibody (REGN2567) to capture anti-GDF8 and Activin A bivalent parental and GDF8 x Activin A bispecific antibodies. Different concentrations of human GDF8 (R&D system), and human Activin A (R&D system), at concentrations ranging from 0.078 nM to 10 nM in a series of 2-fold dilutions prepared in HBS-ET running buffer were injected at a flow rate of 40 μL/min for 4 minutes. The dissociation of human GDF8 and Activin A reagents bound to anti-GDF8 and Activin A bivalent parental and GDF8 x Activin A bispecific antibodies was monitored for 20 minutes in HBS-ET running buffer. At the end of each cycle, the anti-GDF8 and Activin A bivalent parental and GDF8 x Activin A bispecific antibodies capture surface was regenerated using a 12 sec injection of 20 mM H3PO4. The association rate (ka) and dissociation rate (kd) were determined by fitting the real-time binding sensorgrams to a 1:1 binding model with mass transport limitation using Scrubber 2.0c curve-fitting software. Binding dissociation equilibrium constant (K_{D}) and dissociative half-life (t½) were calculated from the kinetic rates as:

$${K}_{D} ( M ) = \frac{kd}{ka} , and t 1 / 2 ( min ) = \frac{ln ( 2 )}{60 * kd}$$

Binding kinetics parameters for human GDF8 and Activin A to the anti-GDF8 and Activin A bivalent parental and GDF8 x Activin A bispecific antibodies of the invention at 25° C. and 37° C. are shown in Table 6 through Table 9.

At 25° C., anti-GDF8 and Activin A bivalent parental and GDF8 x Activin A bispecific antibodies bound to human GDF8 with K_{D} values ranging from 12.6 pM to 17.1 pM, as shown in Table 6.

At 25° C., anti-GDF8 and Activin A bivalent parental and GDF8 x Activin A bispecific antibodies s bound to human Activin A with K_{D} values ranging from 8.66 pM to 202 pM, as shown in Table 7.

At 37° C., anti-GDF8 and Activin A bivalent parental and GDF8 x Activin A bispecific antibodies bound to human GDF8 with K_{D} values ranging from 9.38 pM to 17.8 pM, as shown in Table 8.

At 37° C., anti-GDF8 and Activin A bivalent parental and GDF8 x Activin A bispecific antibodies bound to human Activin A with K_{D} values ranging from 4.29 pM to 446 pM, as shown in Table 9.

[symbol]

### Example 4. Inhibition of Activin a, GDF8, and Combination of Activin a and GDF8 in the Smad 2/3 Luciferase Reporter Assay Using A204/CAGAx12-Luc Cells

The antibodies of the invention were evaluated for their ability to inhibit signaling mediated by activin A, GDF8 or a combination of activin A and GDF8 in a cell-based assay. Activins and GDF8 bind to activin Type IlA and IlB receptors (ActRIIA and ActRIIB, respectively), initiating signaling events leading to phosphorylation-dependent dimerization and activation of SMAD 2/3 transcription factors. A bioassay was developed using a human A204 rhabdomyosarcoma cell line (ATCC, #HTB-82) engineered to express a Smad 2/3-luciferase reporter plasmid (CAGAx12-Luc; Dennler et al, 1998) to create the A204/CAGAx12-Luc cell line.

For the bioassay, A204/CAGAx12-Luc cells were seeded onto 96-well assay plates at 10,000 cells/well in assay media, 0.5% FBS and OPTIMEM (Invitrogen, #31985-070), and incubated at 37° C. and 5% CO₂ overnight. To determine the ligand dose response, ligands were serially diluted in assay media at 1:3 starting from 100 nM to 0.002 nM (activin A, R&D Systems, #338-AC), 200 nM to 0.003 nM (GDF8, R&D Systems, #788-G8/CF) or 50 nM to 0.0008 nM (for each activin A and GDF8 in combination) and added to cells along with a control condition using media containing no ligand. To measure inhibition, antibodies were serially diluted in assay media at 1:3 starting from 100 to 0.002 nM (with an additional well without antibodies) and added to cells with a constant concentration of 100 pM activin A, 1 nM GDF8 or a combination of 50 pM activin A and 50 pM GDF8. After 5.5 hours of incubation in 37° C. and 5% CO₂, OneGlo substrate (Promega, #E6051) was added and then luciferase activity was detected using a Victor X (Perkin Elmer) instrument. The EC₅₀ or IC₅₀ values were determined with GraphPad Prism™ software using nonlinear regression (4-parameter logistics). The percentage of inhibition was calculated based on the relative luminescence unit (RLU) values using the equation:

$$Percent Inhibition = 100 \times \frac{{RLU}_{Baseline} - {RLU}_{Inh}}{{RLU}_{Baseline} - {RLU}_{Background}}$$

In this equation “RLU_{Baseline}” is the value from the cells treated with only ligand without antibodies, “RLUₗₙₕ” are the values from the highest concentration of antibody, and “RLU_{Background}” is the value from cells without ligands or antibodies. Results are shown in Table 10.

[symbol]

As shown in Table 10, four antibodies of the invention, H4H10446P2, H4H10442P2, H4H11418D, and H4H11419D, showed complete inhibition of 100 pM of Activin A with IC₅₀ values of 35.5 pM-3.17 nM and H4H1657N2 showed no inhibition of activin A. Three antibodies, H4H1657N2, H4H11418D, and H4H11419D, showed maximum inhibition ranging 97-99% of 1 nM of GDF8 with IC₅₀ values of 1.48 nM-6.66 nM. H4H10446P2, and H4H10442P2 showed no inhibition of GDF8. H4H11419D showed complete inhibition of combination of activin A and GDF8 with IC₅₀ value of 212 pM whereas H4H10446P2 inhibited with IC₅₀ of 3.97 pM with a maximum inhibition of 57% and H4H1657N2 inhibited with maximum inhibition of 20%. Human IgG4 negative control (REGN1945) showed little to no inhibition (8-12%) of either ligand alone or in combination. Activin A, GDF8 and a combination of Activin A and GDF8 showed activation with EC₅₀ values of 43.8 pM, 115 pM and 34.8 pM, respectively.

### REFERENCE

Dennier S, Itoh S, Vivien D, ten Dijke P, Huet S, Gauthier J M. Direct binding of Smad3 and Smad4 to critical TGF beta-inducible elements in the promoter of human plasminogen activator inhibitor-type 1 gene. EMBO J. 1998 Jun. 1; 17(11):3091-100. doi: 10.1093/emboj/17.11.3091. PMID: 9606191; PMCID: PMC1170648.

### Example 5. In Vivo Effects of Bispecific Antibody on Muscle and Fat Mass in Diet-Induced Obese Mice

The in vivo effects of bispecific antibody H4H11419D (REGN18900) on muscle and fat mass were investigated in diet-induced obese mice. The combination treatment of H4H1657N2 (anti-myostatin antibody) and H4H10446P2 (anti-Activin A antibody) and H4H11419D/REGN18900 (the GDF8 x Activin A bispecific antibody of the invention), were compared head to head in diet-induced obese (DIO) male mice to determine if they have comparable effects on body composition.

H4H11419D was generated by force pairing the heavy chains from H4H1657N2 and H4H10446P2 with the common light chain of H4H1657N2.

Fifty-five, 27-week-old male mice, who were on a 60% high-fat diet for 21 weeks, were separated into five groups of eleven mice each based on their baseline fat mass. The five treatment groups were as follows: single treatment of H4H1657N2 at 20 mg/kg, single treatment of H4H10446P2 at 20 mg/kg, combination treatment of H4H1657N2+H4H10446P2 at 10 mg/kg, H4H11419D at 20 mg/kg, or a human IgG4 isotope control antibody (REGN1945), at 20 mg/kg. Each mouse was given 5 treatment doses over a four-week period. The dosing schedule and groups are listed in Table 11.

TABLE 11
Study groups and dosing

| | | | | Dosing | |
|---|---|---|---|---|---|
| Group # | Treatment | Dose | Route | Schedule | # Mice |
| 1 | Isotype | 20 mg/kg | S.C. | D0, D3, D7, | 11 |
| | control | | | D14, D21 | |
| 2 | H4H1657N2 | 20 mg/kg | S.C. | D0, D3, D7, | 11 |
| | | | | D14, D21 | |
| 3 | H4H10446P2 | 20 mg/kg | S.C. | D0, D3, D7, | 11 |
| | | | | D14, D21 | |
| 4 | H4H1657N2/ | 10 mg/kg + | S.C. | D0, D3, D7, | 11 |
| | H4H10446P2 | 10 mg/kg | | D14, D21 | |
| 5 | H4H11419D | 20 mg/kg | S.C. | D0, D3, D7, | 11 |
| | | | | D14, D21 | |

Body weights were taken throughout the study and body composition was measured by EchoMRI at baseline and on study days 4, 11, 18 and 24. On days 27 and 28, the mice were euthanized in a staggered fashion and the following organ weights were measured: tibialis anterior (TA) muscle, quadriceps muscle, gastrocnemius muscle (GA; dissection also included the soleus), subcutaneous white adipose (scWAT), gonadal white adipose tissue (gWAT), liver and heart.

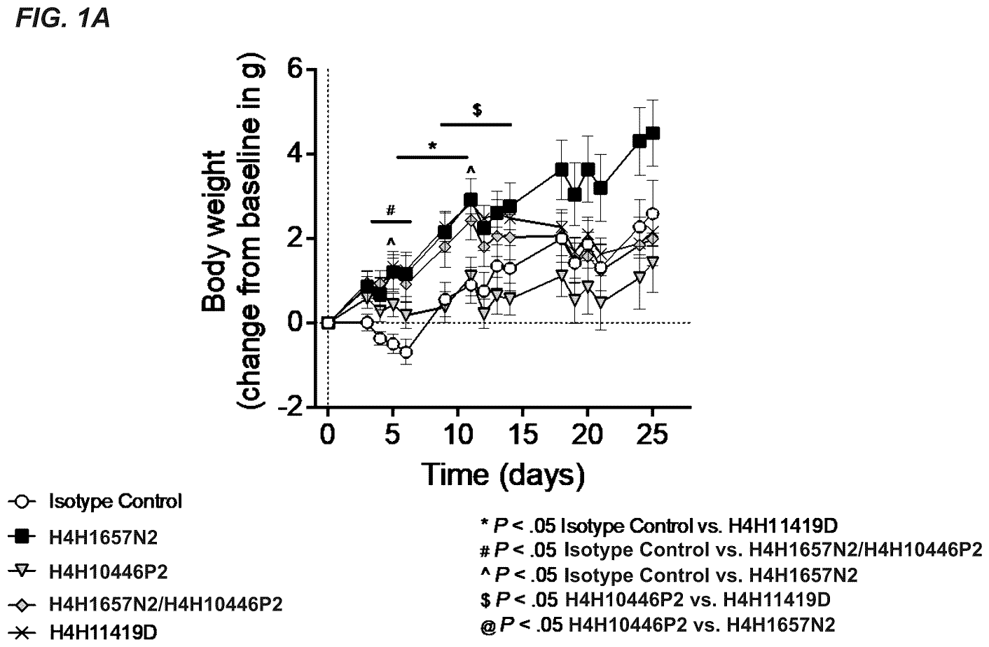

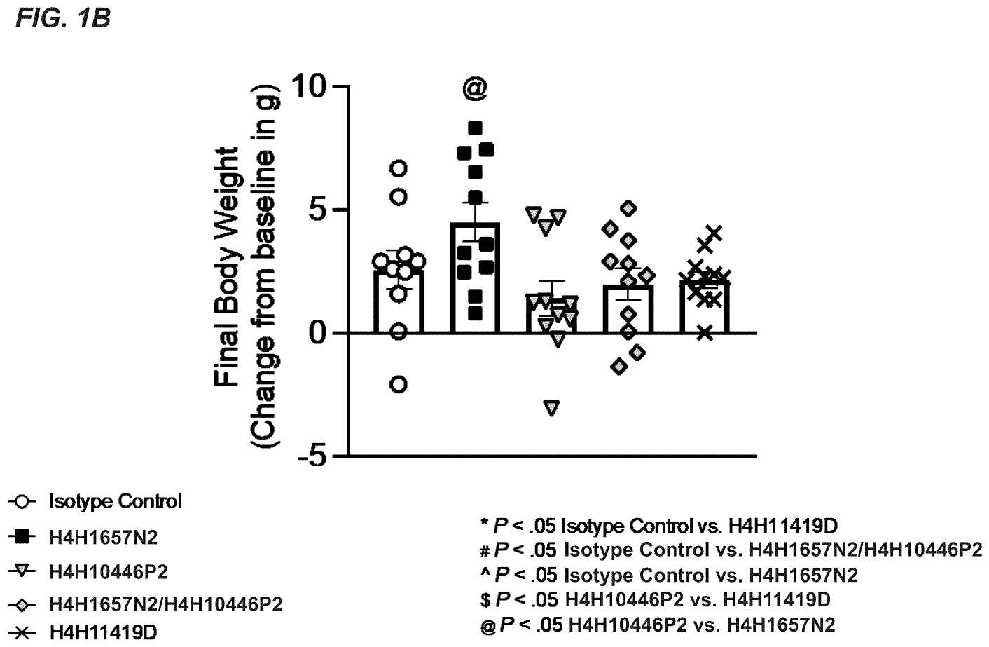

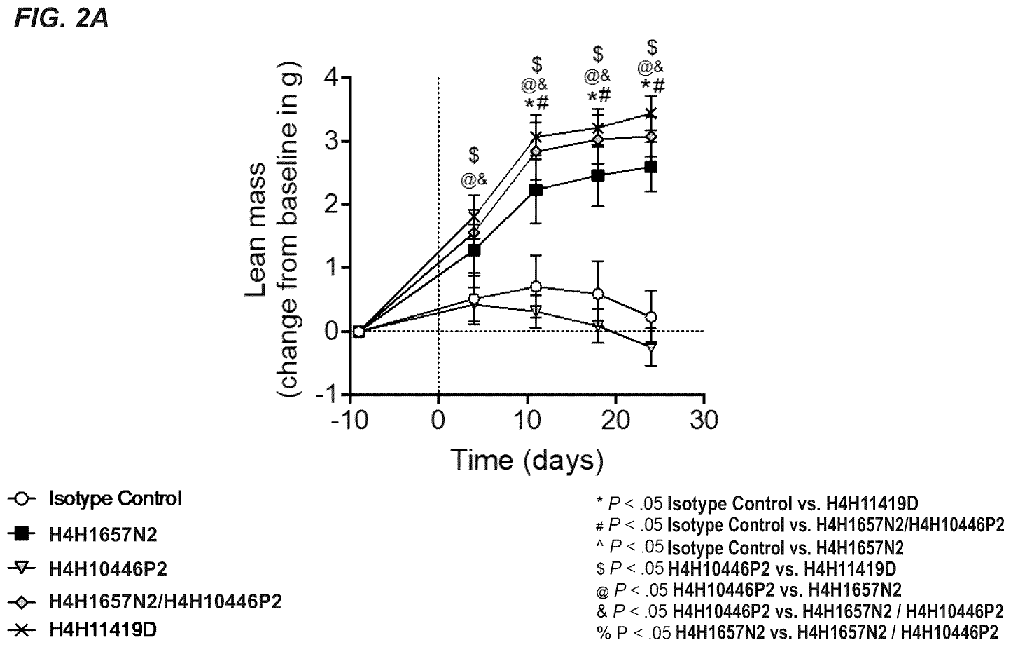

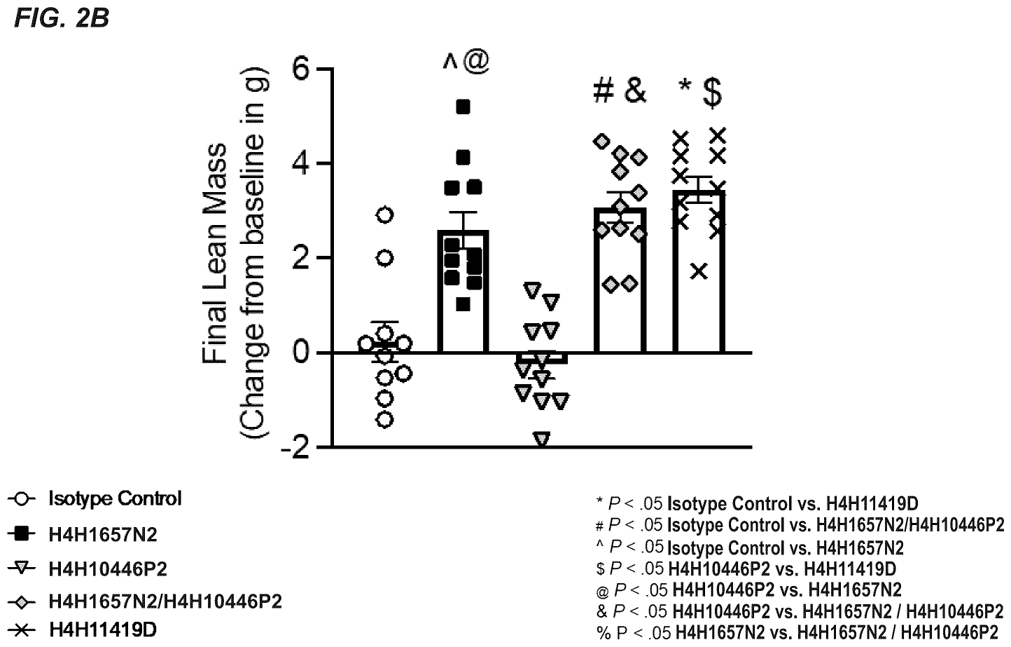

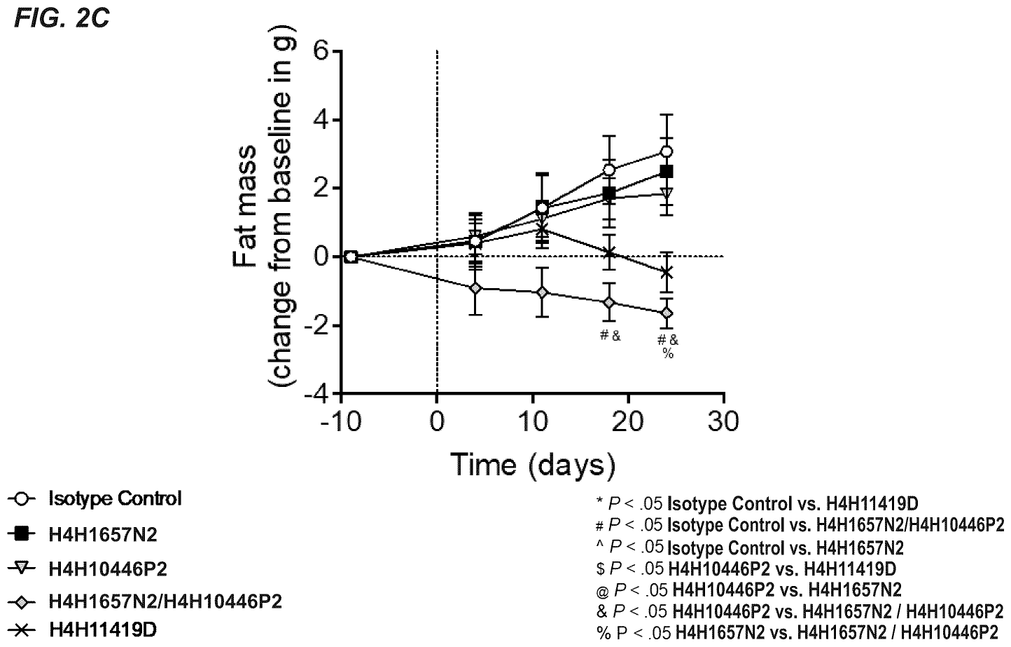

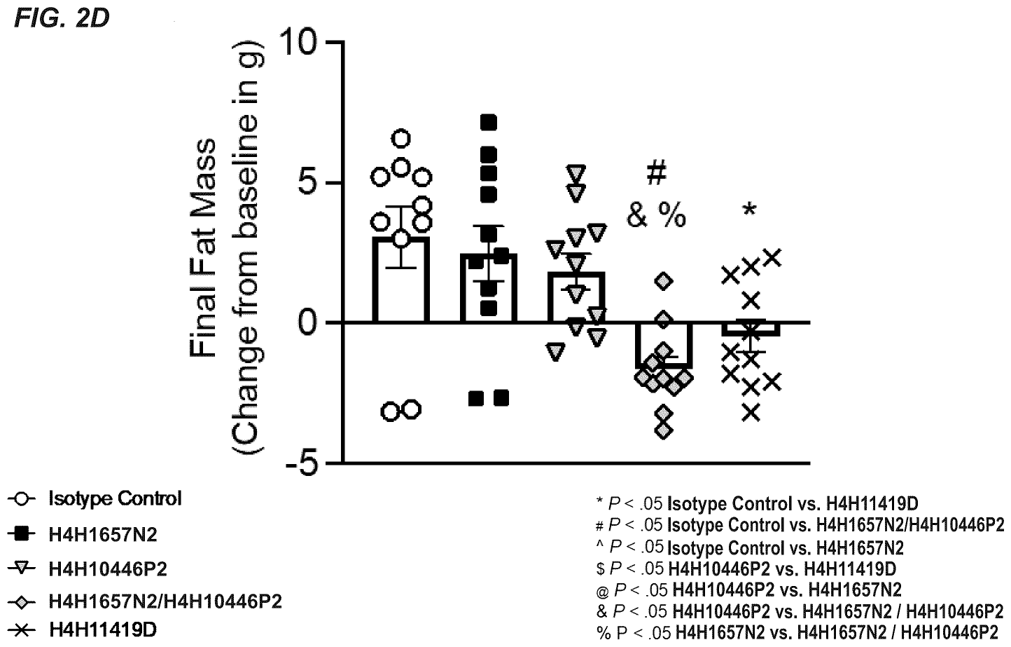

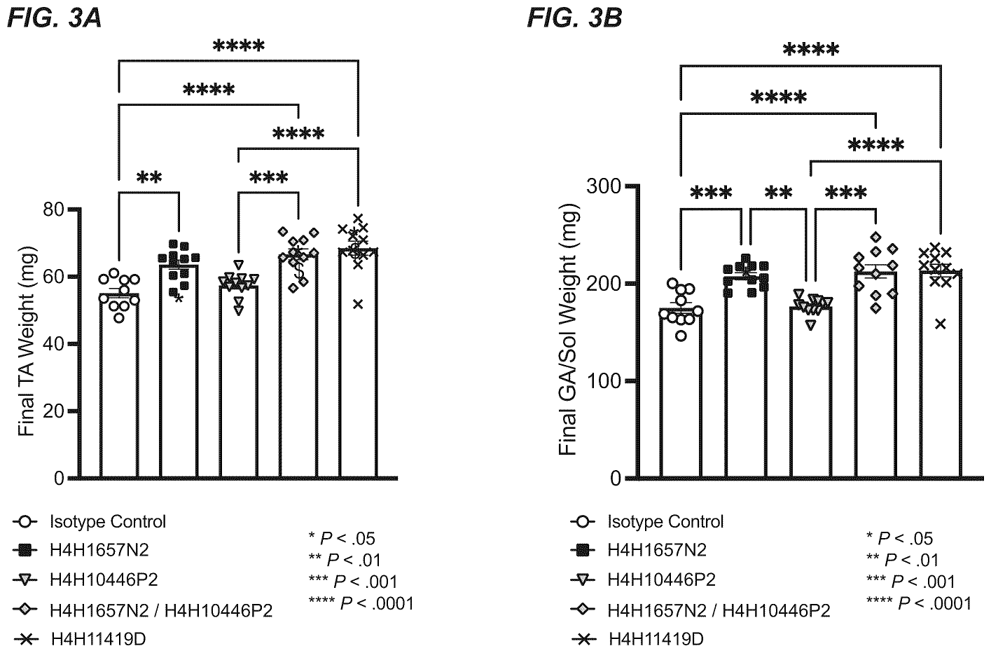

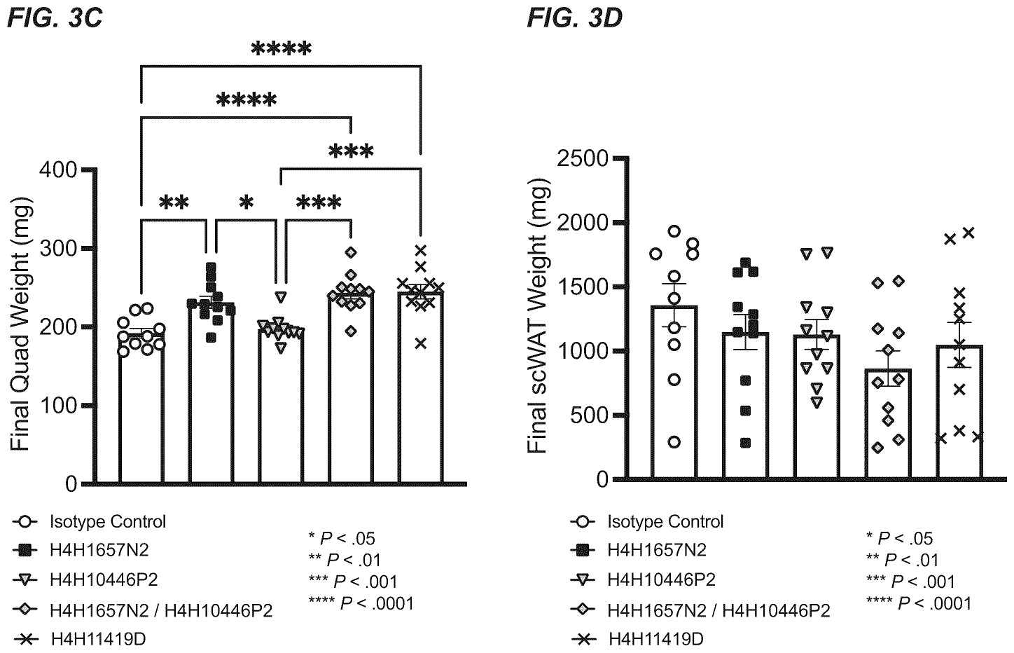

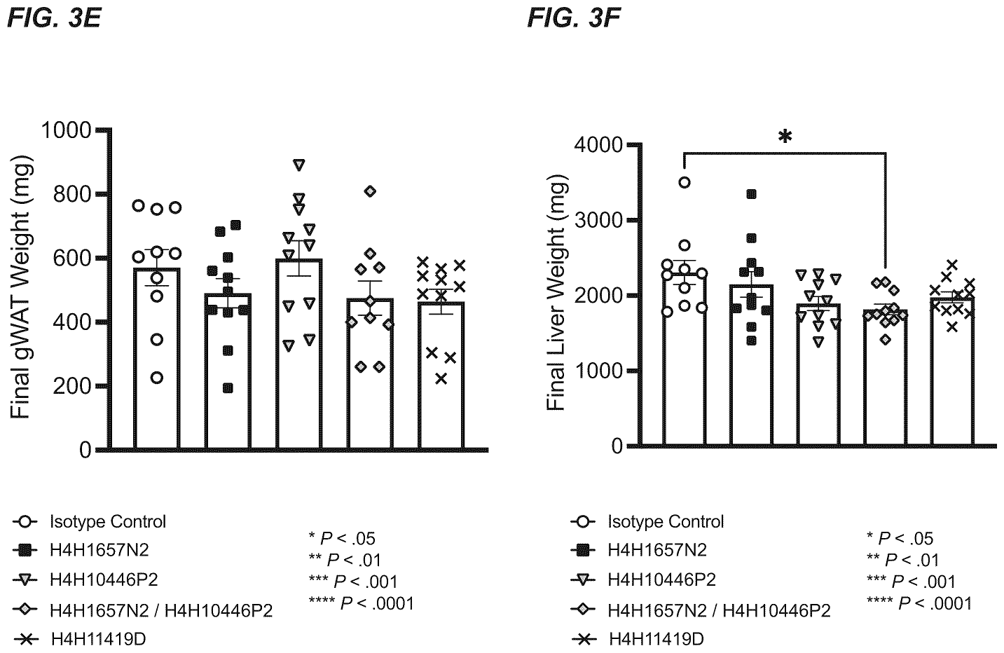

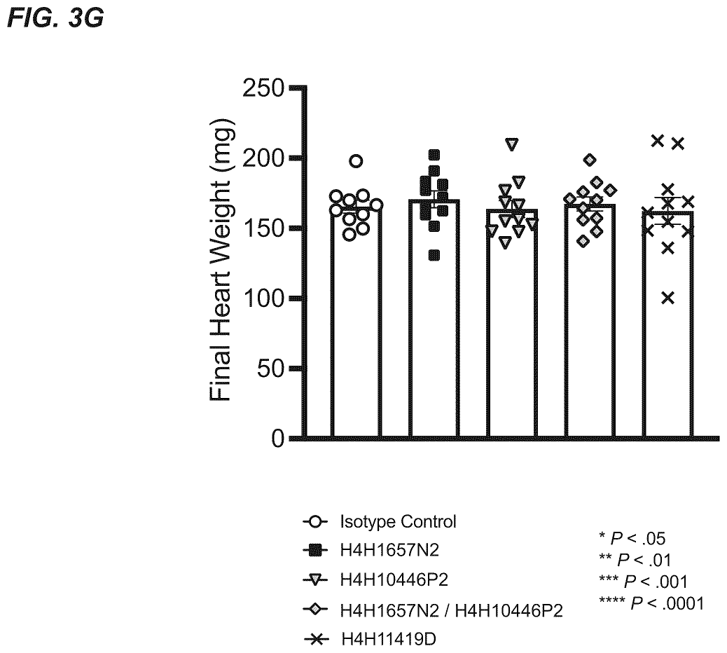

Change in body weight, total body fat and total lean mass were calculated as the difference in grams from each time point compared to each baseline reading. Body weights are shown in FIGS. 1A and B and body composition is shown in FIGS. 2A-D. Each figure shows the difference in body weight over time (A) and the final body weight differences between each group (B). Changes over time were assessed by two-way ANOVA while the final results were compared by one-way ANOVA. Terminal tissue weights, assessed by one-way ANOVA, are shown in FIGS. 3A-G.

Results summary and conclusions: Body weights for all groups increased over the four-week study (FIG. 1A), with the H4H1657N2 monotherapy group having gained the most weight (10.2%, 4.5 g±0.24 g, average+SEM, NS vs REGN1945 control 2.6 g±0.25 g) at the end of four weeks. Body weight for other groups increased similarly to the control at study termination (FIG. 1B, H4H10446P2: 1.4 g±0.21 g; H4H1657N2+H4H10446P2: 2.0 g±0.19 g; H4H11419D: 2.2 g±0.10 g). No group, however, was significantly different from the isotype control group at the end of the study.

Changes in body composition are shown in FIGS. 2A-D. H4H1657N2 monotherapy, H4H1657N2+H4H10446P2 and H4H11419D groups all demonstrated significant lean mass gains (H4H1657N2: 2.6 g±0.12 g, final average lean mass change vs. baseline+SEM; H4H1657N2+H4H10446P2, 3.1 g±0.10 g; H1 H11419D, 3.4 g±0.08 g, all P<0.05 vs. REGN1945 after 4 weeks of treatment, FIG. 2A-B). The isotype control and H4H10446P2 groups did not show any significant gain in lean mass (0.2 g±0.13 g and −0.2 g±0.09 g respectively).

H4H1657N2+H4H10446P2 and H4H11419D groups both showed decreases in total body fat (H4H1657N2+H4H10446P2, −1.6 g±0.13 g; H4H11419D, −0.5 g±0.17 g, both P<0.05 vs. isotype control after 4 weeks of treatment, FIG. 2C-D). The remaining groups all gained similar amounts of fat mass over the four-week study (isotype control, 3.1 g±0.34 g; H4H1657N2: 2.5 g±0.30 g; H4H10446P2: 1.8 g±0.19 g).

The terminal weights of all tissues are shown in FIGS. 3A-G. H4H1657N2 monotherapy, H4H1657N2+H4H10446P2 and H4H11419D all showed significant increases in TA, GA/Sol and Quad muscles vs. isotype control over the four-week period. H4H10446P2 alone did not increase muscle weights. The only other significant change was a significant decrease in liver weight in the H4H1657N2+H4H10446P2 group. Final weights are summarized in Table 12.

In conclusion, the 20 mg/kg dosing of H4H11419D bi-specific antibody works similarly compared to dosing the combination of 10 mg/kg of H4H1657N2+H4H10446P2 combination to increase skeletal muscle weights in diet-induced obese mice.

[symbol]

1. An isolated antigen-binding molecule comprising a first antigen-binding domain that specifically binds to GDF8; and a second antigen-binding domain that specifically binds to Activin A.

2. The isolated antigen-binding molecule of claim 1, further comprising a common light chain.

3. The isolated antigen-binding molecule of claim 1, wherein the isolated antigen-binding molecule specifically binds human GDF8 with a K_{D} of less than about 20 pM, or less than about 15 pM, as measured in a surface plasmon resonance assay at 25° C.

4. The isolated antigen-binding molecule of claim 1, wherein the isolated antigen-binding molecule specifically binds human Activin A with a K_{D} of less than about 250 pM, less than about 225 pM, less than about 100 pM, less than about 50 pM, or less than about 25 pM as measured in a surface plasmon resonance assay at 25° C.

5. The isolated antigen-binding molecule of claim 1, wherein the isolated antigen-binding molecule inhibits signaling mediated by Activin A, GDF8, or a combination of Activin A and GDF8 in a cell-based assay.

6. The isolated antigen-binding molecule of claim 5, wherein the isolated antigen-binding molecule inhibits signaling mediated by Activin A in a cell-based assay with an EC₅₀ of about 5 nM or less.

7. The isolated antigen-binding molecule of claim 5, wherein the isolated antigen-binding molecule inhibits signaling mediated by GDF8 in a cell-based assay with an EC₅₀ of about 10 nM or less.

8. The isolated antigen-binding molecule of claim 1, wherein the isolated antigen-binding molecule blocks activation by Activin A, GDF8, or a combination of Activin A and GDF8 of at least one activin receptor.

9. The isolated antigen-binding molecule of claim 8, wherein the least one activin receptor comprises one or more of an activin Type IIA (ActRIIA) receptor and an activin Type IIB (ActRIIB) receptor.

10. The isolated antigen-binding molecule of claim 1, wherein the isolated antigen-binding molecule has one or more properties selected from the group consisting of: (a) is a fully human monoclonal antibody;
(b) binds to GDF8 protein Asp268-Ser376 of UniProtKB/Swiss-Prot accession number 008689 (SEQ ID NO: 42) at 25° C. with a dissociation constant (K_{D}) of less than about 100 pM, less than about 70 pM, less than about 50 pM, less than about 25 pM, less than about 20 pM, or less than about 15 pM, as measured in a surface plasmon resonance assay; (c) binds to GDF8 protein Asp268-Ser376 of UniProtKB/Swiss-Prot accession number 008689 (SEQ ID NO: 42) at 37° C. with a dissociation constant (K_{D}) of less than about 100 pM, less than about 70 pM, less than about 50 pM, less than about 25 pM, less than about 20 pM, less than about 15 pM, or less than about 12 pM as measured in a surface plasmon resonance assay; (d) binds to Activin A protein Gly311-Ser426 of UniProtKB/Swiss-Prot accession number P08476 (SEQ ID NO: 43) at 25° C. with a dissociation constant (K_{D}) of less than about 250 pM, less than about 225 pM, less than about 100 pM, less than about 50 pM, less than about 25 pM, or less than about 20 pM as measured in a surface plasmon resonance assay; (e) binds to Activin A protein Gly311-Ser426 of UniProtKB/Swiss-Prot accession number P08476 (SEQ ID NO: 43) at 37° C. with a dissociation constant (K_{D}) of less than about 1 nM, less than about 750 pM, less than about 500 pM, less than about 200 pM, less than about 100 pM, lessthan about 50 pM, or less than about 25 pM as measured in a surface plasmon resonance assay; (f) inhibits activation of cells expressing SMAD 2/3 transcription factors by human Activin A with a ICsₒ of less than 5 nM, less than 3 nM, less than 2 nM, less than 1 nM, less than 0.5 nM, less than 0.25 nM, or less than 0.2 nM as measured in a cell-based bioassay; (g) inhibits activation of cells expressing SMAD 2/3 transcription factors by GDF8 with a ICsₒ of less than 20 nM, less than 15 nM, less than 10 nM, less than 8 nM, or less than 7 nM as measured in a cell-based bioassay;
(h) comprises (i) an HCVR of an antibody that binds GDF8, wherein the HCVR comprises an amino acid sequence selected from the group consisting of sequences listed in Table 1, (ii) an HCVR of an antibody that binds Activin A, wherein the HCVR comprises an amino acid sequence selected from the group consisting of sequences listed in Table 1; and (iii) an LCVR of an antibody that binds GDF8, wherein the LCVR comprises an amino acid sequence selected from the group consisting of sequences listed in Table 1; (j) does not include an LCVR of an antibody that binds Activin A; and (k) does not include an LCVR comprising an amino acid sequence selected from the group consisting of LCVR sequences listed in Table 13.

11. The isolated antigen-binding molecule of claim 2, wherein
the first antigen-binding domain that specifically binds to GDF8 comprises three heavy chain complementarity-determining regions (HCDRs) within a heavy chain variable region (HCVR);
the second antigen-binding domain that specifically binds to Activin A comprises three HCDRs within an HCVR; and
the common light chain comprises three light chain complementarity determining regions (LCDRs) within a light chain variable region (LCVR).

12. The isolated antigen-binding molecule of claim 11, comprising (i) three HCDRs of an HCVR of an antibody that binds GDF8; (ii) three HCDRs of an HCVR of an antibody that binds Activin A; and (iii) three LCDRs of an LCVR of an antibody that binds GDF8.

13. The isolated antigen-binding molecule of claim 12, wherein the isolated antigen-binding molecule does not comprise (iv) three LCDRs of an LCVR of an antibody that binds Activin A.

14. The isolated antigen-binding molecule of claim 1, comprising
the first antigen-binding domain that specifically binds to GDF8 comprising a first heavy chain comprising three HCDRs within a first HCVR;
the second antigen-binding domain that specifically binds to Activin A comprising a second heavy chain comprising three HCDRs within a second HCVR; and
a common light chain force paired with the first heavy chain and the second heavy chain, wherein the common light chain comprises three LCDRs within a LCVR.

15. The isolated antigen-binding molecule of claim 14, wherein the first antigen-binding domain that specifically binds to GDF8 comprises three HCDRs within an HCVR comprising the amino acid sequence of SEQ ID NO: 2.

16. The isolated antigen-binding molecule of claim 14, wherein the first antigen-binding domain that specifically binds to GDF8 comprises three heavy chain CDRs: heavy chain CDR1 (HCDR1), HCDR2, and HCDR3 comprising the amino acid sequences of SEQ ID NO: 4, SEQ ID NO: 6, and SEQ ID NO: 8, respectively.

17. The isolated antigen-binding molecule of claim 14, wherein the first antigen-binding domain that specifically binds to GDF8 comprises an HCVR comprising the amino acid sequence of SEQ ID NO: 2.

18. The isolated antigen-binding molecule of claim 14, wherein the second antigen-binding domain that specifically binds to Activin A comprises three HCDRs within an HCVR comprising the amino acid sequence selected from the group consisting of SEQ ID NO: 10 and SEQ ID NO: 32.

19. The isolated antigen-binding molecule of claim 18, wherein the second antigen-binding domain that specifically binds to Activin A comprises three heavy chain CDRs: HCDR1, HCDR2, and HCDR3 comprising the amino acid sequences selected from the group consisting of SEQ ID NOs: 12-14-16, and SEQ ID NOs:12-34-36, respectively.

20. The isolated antigen-binding molecule of claim 14, wherein the second antigen-binding domain that specifically binds to Activin A comprises an HCVR comprising the amino acid sequence selected from the group consisting of SEQ ID NO: 10 and SEQ ID NO: 32.

21. The isolated antigen-binding molecule of claim 14, wherein the common light chain comprises three LCDRs within an LCVR comprising the amino acid sequence of SEQ ID NO: 18.

22. The isolated antigen-binding molecule of claim 14, wherein the common light chain comprises three LCDRs: LCDR1, LCDR2, and LCDR3 comprising the amino acid sequences of SEQ ID NO: 20, SEQ ID NO: 22, and SEQ ID NO: 24, respectively.

23. The isolated antigen-binding molecule of claim 14, wherein the common light chain comprises an LCVR comprising the amino acid sequence of SEQ ID NO: 18.

24. The isolated antigen-binding molecule of claim 2, wherein
the first antigen-binding domain that specifically binds to GDF8 comprises three CDRs within an HCVR comprising the amino acid sequence of SEQ ID NO: 2;
the second antigen-binding domain that specifically binds to Activin A comprises three HCDRs within an HCVR comprising the amino acid sequence selected from the group consisting of SEQ ID NO: 10 and SEQ ID NO: 32; and
the common light chain comprises three LCDRs within an LCVR comprising the amino acid sequence of SEQ ID NO: 18.

25. The isolated antigen-binding molecule of claim 24, wherein
the first antigen-binding domain that specifically binds to GDF8 comprises three HCDRs: HCDR1, HCDR2, and HCDR3 comprising the amino acid sequences of SEQ ID NO: 4, SEQ ID NO: 6, and SEQ ID NO: 8, respectively; and
the second antigen-binding domain that specifically binds to Activin A comprises three HCDRs: HCDR1, HCDR2, and HCDR3 comprising the amino acid sequences selected from the group consisting of SEQ ID NOs: 12-14-16, and SEQ ID NOs:12-34-36, respectively.

26. The isolated antigen-binding molecule of claim 14, wherein the first heavy chain comprises an amino acid sequence of SEQ ID NO: 26.

27. The isolated antigen-binding molecule of claim 14, wherein the second heavy chain comprises an amino acid sequence selected from the group consisting of SEQ ID NO: 28 and 38.

28. The isolated antigen-binding molecule of claim 14, wherein the common light chain comprises the amino acid sequence of SEQ ID NO: 30.

29. An isolated antigen-binding molecule comprising a first antigen-binding domain that specifically binds to GDF8; a second antigen-binding domain that specifically binds to Activin A; and a common light chain.

30. The isolated antigen-binding molecule of claim 29, wherein the common light chain is force paired with both of the first antigen-binding domain that specifically binds to GDF8 and with the second antigen-binding domain that specifically binds to Activin A.

31. The isolated antigen-binding molecule of claim 29, wherein the isolated antigen-binding molecule is a bispecific antibody.

32. A pharmaceutical composition comprising the antigen-binding molecule of claim 1 and a pharmaceutically acceptable carrier.

33. A method for increasing muscle mass or strength in a subject, the method comprising administering to the subject an antigen-binding molecule comprising: a GDF8-specific binding domain; an Activin A-specific binding domain; and optionally a common light chain.

34. The method of claim 33, wherein (i) the GDF8-specific binding domain comprises three HCDRs of an HCVR of an antibody that binds GDF8; (ii) the Activin A-specific binding domain comprises three HCDRs of an HCVR of an antibody that binds Activin A; and (iii) the common light chain comprises three LCDRs of an LCVR of an antibody that binds GDF8.

35. The method of claim 34, wherein the three HCDRs of the HCVR of an antibody that binds GDF8 comprises an amino acid sequence selected from the group consisting of sequences listed in Table 1; the three HCDRs of the HCVR of an antibody that binds Activin A comprises an amino acid sequence selected from the group consisting of sequences listed in Table 1; and/or the three LCDRs of an LCVR of an antibody that binds GDF8 comprises an amino acid sequence selected from the group consisting of sequences listed in Table 1.

36. A method for treating, preventing or ameliorating a disease or disorder characterized by decreased muscle mass or strength, the method comprising administering to a subject in need thereof an antigen-binding molecule comprising a GDF8-specific binding domain; an Activin A-specific binding domain; and optionally a common light chain.

37. The method of claim 36, wherein (i) the GDF8-specific binding domain comprises three HCDRs of an HCVR of an antibody that binds GDF8; (ii) the Activin A-specific binding domain comprises three HCDRs of an HCVR of an antibody that binds Activin A; and (iii) the common light chain comprises three LCDRs of an LCVR of an antibody that binds GDF8.

38. The method of claim 37, wherein the three HCDRs of the HCVR of an antibody that binds GDF8 comprises an amino acid sequence selected from the group consisting of sequences listed in Table 1; the three HCDRs of the HCVR of an antibody that binds Activin A comprises an amino acid sequence selected from the group consisting of sequences listed in Table 1; and/or the three LCDRs of an LCVR of an antibody that binds GDF8 comprises an amino acid sequence selected from the group consisting of sequences listed in Table 1.

39. The method of claim 36, wherein the disease or disorder characterized by decreased muscle mass or strength is selected from the group consisting of sarcopenia, cachexia, muscle injury, ligament injury, muscle wasting/atrophy, cancer, obesity, diabetes, arthritis, multiple sclerosis, muscular dystrophy, amyotrophic lateral sclerosis, Parkinson's disease, osteoporosis, osteoarthritis, osteopenia, and a metabolic syndrome.

40. The method of claim 39, wherein the cachexia is idiopathic or is cachexia secondary to another condition.

41. The method of claim 40, wherein the condition is cancer, chronic renal failure, or chronic obstructive pulmonary disease.

42. The method of claim 39, wherein the muscle wasting/atrophy is caused by or associated with a condition selected from the group consisting of disuse, immobilization, bed rest, injury, medical treatment, surgical intervention and by necessity of mechanical ventilation.

43. The method of claim 42, wherein the surgical intervention is selected from the group consisting of hip fracture, hip replacement, and knee replacement.

44. The method of claim 39, wherein the metabolic syndrome includes a disease or disorder selected from the group consisting of diabetes, obesity, nutritional disorders, organ atrophy, chronic obstructive pulmonary disease, and anorexia.

45. The method of claim 33, further comprising administering a second therapeutically agent.

46. The method of claim 45, wherein the second therapeutically active agent is selected from the group consisting of an anti-Activin A antibody or antigen-binding fragment thereof, an anti-GDF8 antibody or antigen-binding fragment thereof, a growth factor inhibitor, an immunosuppressant, an anti-inflammatory agent, a metabolic inhibitor, a glucagon-like peptide-1 receptor agonist (GLP-1 agonist), an enzyme inhibitor, and a cytotoxic/cytostatic agent.


SEQUENCE LISTING (63 sequences)

SEQ ID NO: 1 - DNA, 360 bp, synthetic construct
  source 1..360: mol_type = other DNA
gaggtgcaggtgttggagtctgggggagacttggtacagcctggggggtccctgagactctcctgtgcagcctctggattcacctttagtgcctatgccatgacctgggtccgccaggctccagggaaggggctggagtgggtctcagctattagtggtagtggtggtagcgcatactacgcagactccgtgaagggccggttcaccatctccagagacaattccaagaacacggtatatctgcagatgaacagcctgagagccgaggacacggccgtatattactgtgcgaaagatggggcctggaaaatgtccggtttggacgtctggggccaagggaccacggtcatcgtctcctca

SEQ ID NO: 2 - protein, 120 aa, synthetic construct
EVQVLESGGDLVQPGGSLRLSCAASGFTFSAYAMTWVRQAPGKGLEWVSAISGSGGSAYYADSVKGRFTISRDNSKNTVYLQMNSLRAEDTAVYYCAKDGAWKMSGLDVWGQGTTVIVSS

SEQ ID NO: 3 - DNA, 24 bp, synthetic construct
  source 1..24: mol_type = other DNA
ggattcacctttagtgcctatgcc

SEQ ID NO: 4 - protein, 8 aa, synthetic construct
GFTFSAYA

SEQ ID NO: 5 - DNA, 24 bp, synthetic construct
  source 1..24: mol_type = other DNA
attagtggtagtggtggtagcgca

SEQ ID NO: 6 - protein, 8 aa, synthetic construct
ISGSGGSA

SEQ ID NO: 7 - DNA, 39 bp, synthetic construct
  source 1..39: mol_type = other DNA
gcgaaagatggggcctggaaaatgtccggtttggacgtc

SEQ ID NO: 8 - protein, 13 aa, synthetic construct
AKDGAWKMSGLDV

SEQ ID NO: 9 - DNA, 378 bp, synthetic construct
  source 1..378: mol_type = other DNA
caggtgcagctgcaggagtcgggcccaggactggtgaagccctcggagaccctgtccatcacctgcactgtctctggtggctccttcagtagtcacttctggacctggatccggcagcccccaggaaagggactggaatggattggatatctccattatagtgggggcaccagctacaacccctccctcaagagtcgagtcatcatatcagtggacacgtccaagaaccagttctccctgaaactgaactctgtgaccgctgcggacacggccgtgtattactgtgcgagagctagatcggggattacttttgggggacttatcgtccctggttcttttgatatctggggccaagggacaatggtcaccgtctcttca

SEQ ID NO: 10 - protein, 126 aa, synthetic construct
QVQLQESGPGLVKPSETLSITCTVSGGSFSSHFWTWIRQPPGKGLEWIGYLHYSGGTSYNPSLKSRVIISVDTSKNQFSLKLNSVTAADTAVYYCARARSGITFGGLIVPGSFDIWGQGTMVTVSS

SEQ ID NO: 11 - DNA, 24 bp, synthetic construct
  source 1..24: mol_type = other DNA
ggtggctccttcagtagtcacttc

SEQ ID NO: 12 - protein, 8 aa, synthetic construct
GGSFSSHF

SEQ ID NO: 13 - DNA, 21 bp, synthetic construct
  source 1..21: mol_type = other DNA
ctccattatagtgggggcacc

SEQ ID NO: 14 - protein, 7 aa, synthetic construct
LHYSGGT

SEQ ID NO: 15 - DNA, 60 bp, synthetic construct
  source 1..60: mol_type = other DNA
gcgagagctagatcggggattacttttgggggacttatcgtccctggttcttttgatatc

SEQ ID NO: 16 - protein, 20 aa, synthetic construct
ARARSGITFGGLIVPGSFDI

SEQ ID NO: 17 - DNA, 321 bp, synthetic construct
  source 1..321: mol_type = other DNA
gacatccagatgacccagtctccagcctccctgtctgcatctgttggagacagagtcaccatcacttgtcgggcgagtcaggacattagcgattatttagcctggtatcagcagaaaccagggaaaattcctaggctcctgatctatactacatccactttgcaatcaggggtcccatctcggttccgtggcagtgggtctgggacagatttcactctcaccatcagcagcctgcagcctgaagatgttgcaacttattactgtcagaagtatgacagtgccccgctcactttcggcggagggaccaaggtggagatcaaa

SEQ ID NO: 18 - protein, 107 aa, synthetic construct
DIQMTQSPASLSASVGDRVTITCRASQDISDYLAWYQQKPGKIPRLLIYTTSTLQSGVPSRFRGSGSGTDFTLTISSLQPEDVATYYCQKYDSAPLTFGGGTKVEIK

SEQ ID NO: 19 - DNA, 18 bp, synthetic construct
  source 1..18: mol_type = other DNA
caggacattagcgattat

SEQ ID NO: 20 - protein, 6 aa, synthetic construct
QDISDY

SEQ ID NO: 21-22 - skipped (2 entries)

SEQ ID NO: 23 - DNA, 27 bp, synthetic construct
  source 1..27: mol_type = other DNA
cagaagtatgacagtgccccgctcact

SEQ ID NO: 24 - protein, 9 aa, synthetic construct
QKYDSAPLT

SEQ ID NO: 25 - DNA, 1344 bp, synthetic construct
  source 1..1344: mol_type = other DNA
gaggtgcaggtgttggagtctgggggagacttggtacagcctggggggtccctgagactctcctgtgcagcctctggattcacctttagtgcctatgccatgacctgggtccgccaggctccagggaaggggctggagtgggtctcagctattagtggtagtggtggtagcgcatactacgcagactccgtgaagggccggttcaccatctccagagacaattccaagaacacggtatatctgcagatgaacagcctgagagccgaggacacggccgtatattactgtgcgaaagatggggcctggaaaatgtccggtttggacgtctggggccaagggaccacggtcatcgtctcctcagcctccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggtaaatga

SEQ ID NO: 26 - protein, 447 aa, synthetic construct
EVQVLESGGDLVQPGGSLRLSCAASGFTFSAYAMTWVRQAPGKGLEWVSAISGSGGSAYYADSVKGRFTISRDNSKNTVYLQMNSLRAEDTAVYYCAKDGAWKMSGLDVWGQGTTVIVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 27 - DNA, 1362 bp, synthetic construct
  source 1..1362: mol_type = other DNA
caggtgcagctgcaggagtcgggcccaggactggtgaagccctcggagaccctgtccatcacctgcactgtctctggtggctccttcagtagtcacttctggacctggatccggcagcccccaggaaagggactggaatggattggatatctccattatagtgggggcaccagctacaacccctccctcaagagtcgagtcatcatatcagtggacacgtccaagaaccagttctccctgaaactgaactctgtgaccgctgcggacacggccgtgtattactgtgcgagagctagatcggggattacttttgggggacttatcgtccctggttcttttgatatctggggccaagggacaatggtcaccgtctcttcagccagcacaaaaggtcctagcgtttttccacttgccccatgttcaaggtcaacctccgaaagtaccgccgctcttggctgtctcgtaaaagattattttcccgaacctgtaactgtctcctggaactccggcgcactcacttccggcgtacataccttccccgctgtcctccaatcttccggtctctactccctgtcttctgttgtcactgttccatcatcctcactcggcacaaaaacatatacctgcaacgttgatcacaagccaagtaataccaaagttgataagcgcgtcgaatccaaatacggtcccccctgccccccatgtcccgctccagagtttctcggtggcccctctgttttcctttttccccctaaacccaaagataccctcatgatttccagaacccccgaggtcacctgcgtcgtcgttgatgtaagccaagaagatcccgaagtccagttcaattggtatgtagacggtgttgaagtccataatgcaaaaacaaaacccagagaggaacagtttaattcaacctatcgtgtcgttagcgtactcaccgttcttcatcaagactggctcaatggaaaagaatataaatgtaaagttagcaacaaaggtctgcccagttcaatcgaaaaaacaattagcaaagccaaaggccaacctcgcgaaccccaagtctataccttgcccccttctcaggaagaaatgaccaaaaaccaagtttcactcacatgcctcgtaaaaggattctatccatcagacattgcagtagaatgggaatctaacggccaacctgaaaataattacaaaaccactcctcctgtcctcgattctgacggctcttttttcctttactccagattgactgttgataaatcccgctggcaggaaggtaacgttttttcttgttctgtgatgcacgaagccctccataacagattcactcaaaaatctctttctctctcccctggcaaataa

SEQ ID NO: 28 - protein, 453 aa, synthetic construct
QVQLQESGPGLVKPSETLSITCTVSGGSFSSHFWTWIRQPPGKGLEWIGYLHYSGGTSYNPSLKSRVIISVDTSKNQFSLKLNSVTAADTAVYYCARARSGITFGGLIVPGSFDIWGQGTMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNRFTQKSLSLSPGK

SEQ ID NO: 29 - DNA, 645 bp, synthetic construct
  source 1..645: mol_type = other DNA
gacatccagatgacccagtctccagcctccctgtctgcatctgttggagacagagtcaccatcacttgtcgggcgagtcaggacattagcgattatttagcctggtatcagcagaaaccagggaaaattcctaggctcctgatctatactacatccactttgcaatcaggggtcccatctcggttccgtggcagtgggtctgggacagatttcactctcaccatcagcagcctgcagcctgaagatgttgcaacttattactgtcagaagtatgacagtgccccgctcactttcggcggagggaccaaggtggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 30 - protein, 214 aa, synthetic construct
DIQMTQSPASLSASVGDRVTITCRASQDISDYLAWYQQKPGKIPRLLIYTTSTLQSGVPSRFRGSGSGTDFTLTISSLQPEDVATYYCQKYDSAPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 31 - DNA, 378 bp, synthetic construct
  source 1..378: mol_type = other DNA
caggtgcagctgcaggagtcgggcccaggactggtgaagccctcggagaccctgtccctcacctgcactgtctctggtggctccttcagtagtcacttctggagctggatccggcagcccccagggaagggactggagtggattggatatatcttatacactgggggcaccagcttcaacccctccctcaagagtcgagtctccatgtcagtgggcacgtccaagaaccagttctccctgaaattgagctctgtgaccgctgcggacacggccgtatattactgtgcgagagctagatcggggataacgtttacgggtattatcgtccctggctcttttgatatctggggccaagggacaatggtcaccgtctcttca

SEQ ID NO: 32 - protein, 126 aa, synthetic construct
QVQLQESGPGLVKPSETLSLTCTVSGGSFSSHFWSWIRQPPGKGLEWIGYILYTGGTSFNPSLKSRVSMSVGTSKNQFSLKLSSVTAADTAVYYCARARSGITFTGIIVPGSFDIWGQGTMVTVSS

SEQ ID NO: 33 - DNA, 21 bp, synthetic construct
  source 1..21: mol_type = other DNA
atcttatacactgggggcacc

SEQ ID NO: 34 - protein, 7 aa, synthetic construct
ILYTGGT

SEQ ID NO: 35 - DNA, 60 bp, synthetic construct
  source 1..60: mol_type = other DNA
gcgagagctagatcggggataacgtttacgggtattatcgtccctggctcttttgatatc

SEQ ID NO: 36 - protein, 20 aa, synthetic construct
ARARSGITFTGIIVPGSFDI

SEQ ID NO: 37 - DNA, 1362 bp, synthetic construct
  source 1..1362: mol_type = other DNA
caggtgcagctgcaggagtcgggcccaggactggtgaagccctcggagaccctgtccctcacctgcactgtctctggtggctccttcagtagtcacttctggagctggatccggcagcccccagggaagggactggagtggattggatatatcttatacactgggggcaccagcttcaacccctccctcaagagtcgagtctccatgtcagtgggcacgtccaagaaccagttctccctgaaattgagctctgtgaccgctgcggacacggccgtatattactgtgcgagagctagatcggggataacgtttacgggtattatcgtccctggctcttttgatatctggggccaagggacaatggtcaccgtctcttcagccagcacaaaaggtcctagcgtttttccacttgccccatgttcaaggtcaacctccgaaagtaccgccgctcttggctgtctcgtaaaagattattttcccgaacctgtaactgtctcctggaactccggcgcactcacttccggcgtacataccttccccgctgtcctccaatcttccggtctctactccctgtcttctgttgtcactgttccatcatcctcactcggcacaaaaacatatacctgcaacgttgatcacaagccaagtaataccaaagttgataagcgcgtcgaatccaaatacggtcccccctgccccccatgtcccgctccagagtttctcggtggcccctctgttttcctttttccccctaaacccaaagataccctcatgatttccagaacccccgaggtcacctgcgtcgtcgttgatgtaagccaagaagatcccgaagtccagttcaattggtatgtagacggtgttgaagtccataatgcaaaaacaaaacccagagaggaacagtttaattcaacctatcgtgtcgttagcgtactcaccgttcttcatcaagactggctcaatggaaaagaatataaatgtaaagttagcaacaaaggtctgcccagttcaatcgaaaaaacaattagcaaagccaaaggccaacctcgcgaaccccaagtctataccttgcccccttctcaggaagaaatgaccaaaaaccaagtttcactcacatgcctcgtaaaaggattctatccatcagacattgcagtagaatgggaatctaacggccaacctgaaaataattacaaaaccactcctcctgtcctcgattctgacggctcttttttcctttactccagattgactgttgataaatcccgctggcaggaaggtaacgttttttcttgttctgtgatgcacgaagccctccataacagattcactcaaaaatctctttctctctcccctggcaaataa

SEQ ID NO: 38 - protein, 453 aa, synthetic construct
QVQLQESGPGLVKPSETLSLTCTVSGGSFSSHFWSWIRQPPGKGLEWIGYILYTGGTSFNPSLKSRVSMSVGTSKNQFSLKLSSVTAADTAVYYCARARSGITFTGIIVPGSFDIWGQGTMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNRFTQKSLSLSPGK

SEQ ID NO: 39 - protein, 376 aa, Mus musculus
MMQKLQMYVYIYLFMLIAAGPVDLNEGSEREENVEKEGLCNACAWRQNTRYSRIEAIKIQILSKLRLETAPNISKDAIRQLLPRAPPLRELIDQYDVQRDDSSDGSLEDDDYHATTETIITMPTESDFLMQADGKPKCCFFKFSSKIQYNKVVKAQLWIYLRPVKTPTTVFVQILRLIKPMKDGTRYTGIRSLKLDMSPGTGIWQSIDVKTVLQNWLKQPESNLGIEIKALDENGHDLAVTFPGPGEDGLNPFLEVKVTDTPKRSRRDFGLDCDEHSTESRCCRYPLTVDFEAFGWDWIIAPKRYKANYCSGECEFVFLQKYPHTHLVHQANPRGSAGPCCTPTKMSPINMLYFNGKEQIIYGKIPAMVVDRCGCS

SEQ ID NO: 40 - protein, 426 aa, Homo sapiens
MPLLWLRGFLLASCWIIVRSSPTPGSEGHSAAPDCPSCALAALPKDVPNSQPEMVEAVKKHILNMLHLKKRPDVTQPVPKAALLNAIRKLHVGKVGENGYVEIEDDIGRRAEMNELMEQTSEIITFAESGTARKTLHFEISKEGSDLSVVERAEVWLFLKVPKANRTRTKVTIRLFQQQKHPQGSLDTGEEAEEVGLKGERSELLLSEKVVDARKSTWHVFPVSSSIQRLLDQGKSSLDVRIACEQCQESGASLVLLGKKKKKEEEGEGKKKGGGEGGAGADEEKEQSHRPFLMLQARQSEDHPHRRRRRGLECDGKVNICCKKQFFVSFKDIGWNDWIIAPSGYHANYCEGECPSHIAGTSGSSLSFHSTVINHYRMRGHSPFANLKSCCVPTKLRPMSMLYYDDGQNIIKKDIQNMIVEECGCS

SEQ ID NO: 41 - protein, 407 aa, Homo sapiens [sequence omitted]

SEQ ID NO: 42 - protein, 109 aa, Homo sapiens
DFGLDCDEHSTESRCCRYPLTVDFEAFGWDWIIAPKRYKANYCSGECEFVFLQKYPHTHLVHQANPRGSAGPCCTPTKMSPINMLYFNGKEQIIYGKIPAMVVDRCGCS

SEQ ID NO: 43 - protein, 116 aa, Homo sapiens [sequence omitted]

SEQ ID NO: 44 - protein, 108 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPWTFGQGTKVEIK

SEQ ID NO: 45 - protein, 7 aa, synthetic construct
QSVSSSY

SEQ ID NO: 46 - skipped

SEQ ID NO: 47 - protein, 9 aa, synthetic construct
QQYGSSPWT

SEQ ID NO: 48 - protein, 125 aa, synthetic construct
EVQLVESGGGLVQPGRSLRLSCKASGFAFDDFAMHWVRQAPGKGLEWVSGIVWNSGDIGYADSVKGRFTISRDNAKNSLYLQLNSLRTEDTALYFCVKDMVRGLMGFNYYGMDVWGQGTTVTVSS

SEQ ID NO: 49 - protein, 107 aa, synthetic construct
EIVLTQSPATLSLSPGERATLSCRASQTISTYLVWYRQRPGQAPSLLIYDASNRATDIPARFSGSGSGTDFTLTISSLEPEDFAVYYCQQRSNWPITFGQGTRLEIK

SEQ ID NO: 50 - protein, 8 aa, synthetic construct
GFAFDDFA

SEQ ID NO: 51 - protein, 8 aa, synthetic construct
IVWNSGDI

SEQ ID NO: 52 - protein, 18 aa, synthetic construct
VKDMVRGLMGFNYYGMDV

SEQ ID NO: 53 - protein, 6 aa, synthetic construct
QTISTY

SEQ ID NO: 54 - skipped

SEQ ID NO: 55 - protein, 9 aa, synthetic construct
QQRSNWPIT

SEQ ID NO: 56 - protein, 117 aa, synthetic construct
QVQLVESGGGVVQPGRSLRLTCVASGFTVSSYGMHWVRQAPGKGLEWVAVMFYDESKKYYADSVKGRFTISRDNSKNTLYLQMNSLRVEDTAVYYCARDEQLDFEYWGQGTLVTVSS

SEQ ID NO: 57 - protein, 108 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIK

SEQ ID NO: 58 - protein, 8 aa, synthetic construct
GFTVSSYG

SEQ ID NO: 59 - protein, 8 aa, synthetic construct
MFYDESKK

SEQ ID NO: 60 - protein, 10 aa, synthetic construct
ARDEQLDFEY

SEQ ID NO: 61 - protein, 6 aa, synthetic construct
QSISSY

SEQ ID NO: 62 - skipped

SEQ ID NO: 63 - protein, 10 aa, synthetic construct
QQSYSTPPIT